SEQUENCE LISTING (23 sequences)

SEQ ID NO: 1 - RNA, 1182 nt, Homo sapiens
auggaggagccgcagucagauccuagcgucgagcccccucugagucaggaaacauuuucagaccuauggaaacuacuuccugaaaacaacguucugucccccuugccgucccaagcaauggaugauuugaugcuguccccggacgauauugaacaaugguucacugaagacccagguccagaugaagcucccagaaugccagaggcugcuccccccguggccccugcaccagcagcuccuacaccggcggccccugcaccagcccccuccuggccccugucaucuucugucccuucccagaaaaccuaccagggcagcuacgguuuccgucugggcuucuugcauucugggacagccaagucugugacuugcacguacuccccugcccucaacaagauguuuugccaacuggccaagaccugcccugugcagcuguggguugauuccacacccccgcccggcacccgcguccgcgccauggccaucuacaagcagucacagcacaugacggagguugugaggcgcugcccccaccaugagcgcugcucagauagcgauggucuggccccuccucagcaucuuauccgaguggaaggaaauuugcguguggaguauuuggaugacagaaacacuuuucgacauagugugguggugcccuaugagccgccugagguuggcucugacuguaccaccauccacuacaacuacauguguaacaguuccugcaugggcggcaugaaccggaggcccauccucaccaucaucacacuggaagacuccagugguaaucuacugggacggaacagcuuugaggugcguguuugugccuguccugggagagaccggcgcacagaggaagagaaucuccgcaagaaaggggagccucaccacgagcugcccccagggagcacuaagcgagcacugcccaacaacaccagcuccucuccccagccaaagaagaaaccacuggauggagaauauuucacccuucagauccgugggcgugagcgcuucgagauguuccgagagcugaaugaggccuuggaacucaaggaugcccaggcugggaaggagccaggggggagcagggcucacuccagccaccugaaguccaaaaagggucagucuaccucccgccauaaaaaacucauguucaagacagaagggccugacucagacuga

SEQ ID NO: 2 - RNA, 1182 nt, Artificial Sequence
  Mutant human p53-R175H-mRNA ORF sequence
auggaggagccgcagucagauccuagcgucgagcccccucugagucaggaaacauuuucagaccuauggaaacuacuuccugaaaacaacguucugucccccuugccgucccaagcaauggaugauuugaugcuguccccggacgauauugaacaaugguucacugaagacccagguccagaugaagcucccagaaugccagaggcugcuccccgcguggccccugcaccagcagcuccuacaccggcggccccugcaccagcccccuccuggccccugucaucuucugucccuucccagaaaaccuaccagggcagcuacgguuuccgucugggcuucuugcauucugggacagccaagucugugacuugcacguacuccccugcccucaacaagauguuuugccaacuggccaagaccugcccugugcagcuguggguugauuccacacccccgcccggcacccgcguccgcgccauggccaucuacaagcagucacagcacaugacggagguugugaggcacugcccccaccaugagcgcugcucagauagcgauggucuggccccuccucagcaucuuauccgaguggaaggaaauuugcguguggaguauuuggaugacagaaacacuuuucgacauagugugguggugcccuaugagccgccugagguuggcucugacuguaccaccauccacuacaacuacauguguaacaguuccugcaugggcggcaugaaccggaggcccauccucaccaucaucacacuggaagacuccagugguaaucuacugggacggaacagcuuugaggugcauguuugugccuguccugggagagaccggcgcacagaggaagagaaucuccgcaagaaaggggagccucaccacgagcugcccccagggagcacuaagcgagcacuguccaacaacaccagcuccucuccccagccaaagaagaaaccacuggauggagaauauuucacccuucagauccgugggcgugagcgcuucgagauguuccgagagcugaaugaggccuuggaacucaaggaugcccaggcugggaaggagccaggggggagcagggcucacuccagccaccugaaguccaaaaagggucagucuaccucccgccauaaaaaacucauguucaagacagaagggccugacucagacuga

SEQ ID NO: 3 - RNA, 1173 nt, Mus musculus
augacugccauggaggagucacagucggauaucagccucgagcucccucugagccaggagacauuuucaggcuuauggaaacuacuuccuccagaagauauccugccaucaccucacugcauggacgaucuguugcugccccaggauguugaggaguuuuuugaaggcccaagugaagcccuccgagugucaggagcuccugcagcacaggacccugucaccgagaccccugggccaguggccccugccccagccacuccauggccccugucaucuuuugucccuucucaaaaaacuuaccagggcaacuauggcuuccaccugggcuuccugcagucugggacagccaagucuguuaugugcacguacucuccuccccucaauaagcuauucugccagcuggcgaagacgugcccugugcaguugugggucagcgccacaccuccagcugggagccguguccgcgccauggccaucuacaagaagucacagcacaugacggaggucgugagacgcugcccccaccaugagcgcugcuccgauggugauggccuggcuccuccccagcaucuuauccggguggaaggaaauuuguaucccgaguaucuggaagacaggcagacuuuucgccacagcguggugguaccuuaugagccacccgaggccggcucugaguauaccaccauccacuacaaguacauguguaauagcuccugcauggggggcaugaaccgccgaccuauccuuaccaucaucacacuggaagacuccagugggaaccuucugggacgggacagcuuugagguucguguuugugccugcccugggagagaccgccguacagaagaagaaaauuuccgcaaaaaggaaguccuuugcccugaacugcccccagggagcgcaaagagagcgcugcccaccugcacaagcgccucucccccgcaaaagaaaaaaccacuugauggagaguauuucacccucaagauccgcgggcguaaacgcuucgagauguuccgggagcugaaugaggccuuagaguuaaaggaugcccaugcuacagaggagucuggagacagcagggcucacuccagcuaccugaagaccaagaagggccagucuacuucccgccauaaaaaaacaauggucaagaaaguggggccugacucagacuga

SEQ ID NO: 4 - DNA, 20 bp, Artificial Sequence
  GAPDH Forward primer
ccatggggaaggtgaaggtc

SEQ ID NO: 5 - DNA, 20 bp, Artificial Sequence
  GAPDH Reverse primer
agtgatggcatggactgtgg

SEQ ID NO: 6 - DNA, 26 bp, Artificial Sequence
  p53 Forward primer
atggaggagccgcagtcagatcctag

SEQ ID NO: 7 - DNA, 21 bp, Artificial Sequence
  P53 Reverse primer
tcagtctgagtcaggcccttc

SEQ ID NO: 8 - DNA, 20 bp, Artificial Sequence
  ULK1 Forward primer
tccggattcggattagcagc

SEQ ID NO: 9 - DNA, 20 bp, Artificial Sequence
  ULK1 Reverse primer
ggagaactcgaacttgccca

SEQ ID NO: 10 - DNA, 20 bp, Artificial Sequence
  ATG7 Forward primer
acccagaagaagctgaacga

SEQ ID NO: 11 - DNA, 20 bp, Artificial Sequence
  ATG7 Reverse primer
ctcatttgctgcttgttcca

SEQ ID NO: 12 - DNA, 20 bp, Artificial Sequence
  BECN1 Forward primer
gaagttttccggcggctacc

SEQ ID NO: 13 - DNA, 20 bp, Artificial Sequence
  BECN1 Reverse primer
ctcagcccccgatgctcttc

SEQ ID NO: 14 - DNA, 20 bp, Artificial Sequence
  ATG12 Forward primer
aagtgggcagtagagcgaac

SEQ ID NO: 15 - DNA, 20 bp, Artificial Sequence
  ATG12 Reverse primer
cacgcctgagacttgcagta

SEQ ID NO: 16 - DNA, 20 bp, Artificial Sequence
  TIGAR Forward primer
aagcagagcctgtcgcttag

SEQ ID NO: 17 - DNA, 20 bp, Artificial Sequence
  TIGAR Reverse primer
gcaccaccgctctactgaat

SEQ ID NO: 18 - DNA, 20 bp, Artificial Sequence
  DRAM1 Forward primer
agttcggggtagctcctcat

SEQ ID NO: 19 - DNA, 20 bp, Artificial Sequence
  DRAM1 Reverse primer
gagtcgcagtgaacccagaa

SEQ ID NO: 20 - DNA, 20 bp, Artificial Sequence
  ISG20L1 Forward primer
cgtgcagaccggaagagaca

SEQ ID NO: 21 - DNA, 20 bp, Artificial Sequence
  ISG20L1 Reverse primer
gtggacatacttgagcgcct

SEQ ID NO: 22 - DNA, 20 bp, Artificial Sequence
  SESN1 Forward primer
acggatttgacagctccaca

SEQ ID NO: 23 - DNA, 20 bp, Artificial Sequence
  SESN1 Reverse primer
acccatccgaagactcggta

(19) United States
(12) Patent Application Publication
(10) Pub. No.: US 2022/0016271 A1
(43) Pub. Date: Jan. 20, 2022

(54) METHODS FOR TREATING CANCER

(71) Applicant: The Brigham and Women`s Hospital, Inc., Boston, MA (US)

(72) Inventors: Omid C. Farokhzad, Waban, MA (US); Na Kong, Boston, MA (US); Jinjun Shi, Boston, MA (US); Wei Tao, Brookline, MA (US)

(21) Appl. No.: 17/413,061

(22) PCT Filed: Dec. 11, 2019
(86) PCT No.: PCT/US2019/065740

§ 371 Date: Jun. 11, 2021

Related U.S. Application Data
(60) Provisional application No. 62/778,215, filed on Dec. 11, 2018.

(51) Int. Cl. A61K 51/04; A61K 33/243; A61K 31/436; A61K 31/155; A61K 38/17

(52) U.S. Cl. CPC A61K 51/0408; A61K 33/243; A61K 38/1758; A61K 31/155; A61K 31/436

(57) ABSTRACT

The present application provides a method of treating a cancer, including administering to a subject in need of cancer treatment a therapeutically effective amount of an mRNA encoding tumor suppressor protein p53 in combination with an anticancer therapeutic agent, or a pharmaceutically acceptable salt thereof, wherein the anticancer therapeutic agent is selected from an mTOR inhibitor, a platinum-based antineoplastic agent, and an AMPK activating agent.

### CLAIM OF PRIORITY

This application claims priority to U.S. Patent Application Ser. No. 62/778,215, filed on Dec. 11, 2018, the entire contents of which are hereby incorporated by reference.

### FEDERALLY SPONSORED RESEARCH OR DEVELOPMENT

This invention was made with government support under Grant No. CA200900 awarded by the National Institutes of Health. The government has certain rights in the invention.

### TECHNICAL FIELD

This invention relates to treating cancer, and more specifically to using a combination of p53-encoding mRNA and an mTOR inhibitor, a platinum-based anticancer agent, or an AMPK activator, or a pharmaceutically acceptable salt thereof.

### BACKGROUND

Cancer is one of the leading causes of death in contemporary society. The numbers of new cancer cases and deaths is increasing each year. Currently, cancer incidence is 454.8 cases of cancer per 100,000 men and women per year, while cancer mortality is 71.2 cancer deaths per 100,000 men and women per year. Pharmacological interventions that are safe over the long term may improve cancer treatment and decrease cancer mortality.

### SUMMARY

Loss of function in tumor suppressor genes is commonly associated with the onset/progression of cancer and treatment resistance. The p53 tumor suppressor gene, a master regulator of diverse cellular pathways, is frequently altered in various cancers, for example in ˜36% of hepatocellular carcinomas (HCCs) and ˜68% of non-small cell lung cancers (NSCLCs). Current methods for restoration of p53 expression, including small molecules and DNA therapies, have yielded progressive success but each has formidable drawbacks. In some embodiments, the present disclosure provides a redox-responsive nanoparticle (NP) platform for effective delivery of p53-encoding synthetic messenger RNA (mRNA). The experimental results provided herein demonstrate that the synthetic p53-mRNA NPs drastically delay the growth of p53-null HCC and NSCLC cells by inducing cell cycle arrest and apoptosis. In addition, p53 restoration markedly improves the sensitivity of these tumor cells to everolimus, a mammalian target of rapamycin (mTOR) inhibitor that failed to show clinical benefits in advanced HCC and NSCLC. Moreover, co-targeting of tumor-suppressing p53 and tumorigenic mTOR signaling pathways results in marked anti-tumor effects in vitro and in multiple animal models of HCC and NSCLC.

In one general aspect, the present disclosure provides a method of treating a cancer, the method comprising administering to a subject in need thereof a therapeutically effective amount of an mRNA encoding tumor suppressor protein p53 in combination with an anticancer therapeutic agent, or a pharmaceutically acceptable salt thereof, wherein the anticancer therapeutic agent is selected from an mTOR inhibitor, a platinum-based antineoplastic agent, and an AMPK activating agent.

In some embodiments, the p53-encoding mRNA is within a delivery vehicle capable of providing release of the p53-encoding mRNA in the cancer cell.

In some embodiments, the delivery vehicle is a particle comprising:
a water-insoluble polymeric core; and
the p53-encoding mRNA and a complexing agent within the core.

In some embodiments, the particle further comprises a shell comprising at least one amphiphilic material surrounding the water-insoluble polymeric core.

In some embodiments, the water-insoluble polymeric core comprises one or more polymers selected from a poly(lactic acid), a poly(glycolic acid), and a copolymer of lactic acid and glycolic acid.

In some embodiments, the water-insoluble polymer comprises at least one repeating unit according to Formula (I) or Formula (II):

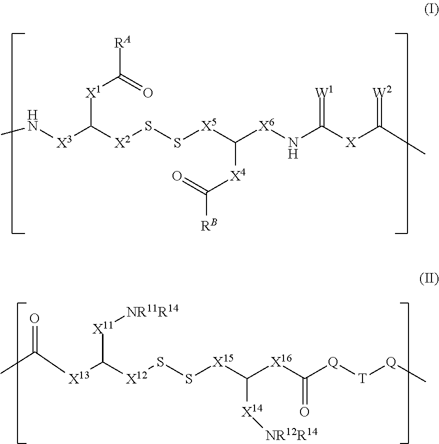

wherein:

X¹ is a bond or C₁₋₁₀₀ alkylene;

X² is C₁₋₁₀₀ alkylene;

X³ is a bond or C₁₋₁₀₀ alkylene;

X⁴ is a bond or C₁₋₁₀₀ alkylene;

X⁵ is C₁₋₁₀₀ alkylene;

X⁶ is a bond or C₁₋₁₀₀ alkylene;

R^{A} is OR¹ or NR³R⁴;

R^{B} is OR² or NR²R⁴;

R¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₁₀₀ alkyl or C(═O)R⁶;

each R⁴ is independently H or C₁₋₁₀₀ alkyl;

each R⁵ is independently H or C₁₋₁₀₀ alkyl;

each R⁶ is independently H or C₁₋₁₀₀ alkyl;

W¹ is O, S, or NH;

W² is O, S, or NH;

X is C₁₋₁₀₀ alkylene, C₂₋₁₀₀ alkenylene, or C₂₋₁₀₀ alkynylene;

provided that when W¹ and W² are both O, then X is C₃₋₁₀₀ alkylene, C₂₋₁₀₀ alkenylene, or C₂₋₁₀₀ alkynylene;

each m is 0, 1 or 2;

X¹¹ is a bond or C₁₋₁₀₀ alkylene;

X¹² is C₁₋₁₀₀ alkylene;

X¹³ is a bond or C₁₋₁₀₀ alkylene;

X¹⁴ is a bond or C₁₋₁₀₀ alkylene;

X¹⁵ is C₁₋₁₀₀ alkylene;

X¹⁶ is a bond or C₁₋₁₀₀ alkylene;

R¹¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;

R¹² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;

each R¹³ is independently H, C₁₋₁₀₀ alkyl or C(═O)R¹⁶;

each R¹⁴ is independently H or C₁₋₁₀₀ alkyl;

each R¹⁵ is independently H or C₁₋₁₀₀ alkyl;

each R¹⁶ is independently H or C₁₋₁₀₀ alkyl;

each Q is independently O or NR¹⁷;

each R¹⁷ is H or C₁₋₁₀₀ alkyl;

T is C₂₋₁₀₀ alkylene, C₄₋₁₀₀ alkenylene, or C₄₋₁₀₀ alkynylene; and

each n is 0, 1 or 2.

In some embodiments, the water-insoluble polymer comprises at least one repeating unit according to Formula (I), wherein:

X¹ is a bond or C₁₋₄ alkylene;

X² is C₁₋₄ alkylene;

X³ is a bond or C₁₋₄ alkylene;

X⁴ is a bond or C₁₋₄ alkylene;

X⁵ is C₁₋₄ alkylene;

X⁶ is a bond or C₁₋₄ alkylene;

R^{A} is OR¹ or NR³R⁴;

R^{B} is OR² or NR²R⁴;

R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₆ alkyl or C(═O)R⁶;

each R⁴ is independently H or C₁₋₆ alkyl;

each R⁵ is independently H or C₁₋₆ alkyl;

each R⁶ is independently H or C₁₋₆ alkyl;

W¹ is O, S, or NH;

W² is O, S, or NH;

X is C₂₋₂₀ alkylene, C₂₋₂₀ alkenylene, or C₂₋₂₀ alkynylene;

provided that when W¹ and W² are both O, then X is C₃₋₂₀ alkylene, C₂₋₂₀ alkenylene, or C₂₋₂₀ alkynylene; and

each m is 0, 1 or 2.

In some embodiments, the water-insoluble polymer comprises at least one repeating unit according to Formula (Ia):

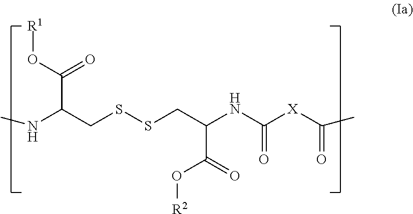

wherein:

R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₆ alkyl or C(═O)R⁶;

each R⁴ is independently H or C₁₋₆ alkyl;

each R⁵ is independently H or C₁₋₆ alkyl;

each R⁶ is independently H or C₁₋₆ alkyl;

X is C₃₋₂₀ alkylene, C₂₋₂₀ alkenylene, or C₂₋₂₀ alkynylene; and

each m is 0, 1 or 2.

In some embodiments:

R¹ is H, C₁₋₂₀ alkyl, C₂₋₂₀ alkenyl, C₂₋₂₀ alkynyl, C₃₋₁₀ cycloalkyl, or C₆₋₁₀ aryl;

R² is H, C₁₋₂₀ alkyl, C₂₋₂₀ alkenyl, C₂₋₂₀ alkynyl, C₃₋₁₀ cycloalkyl, or C₆₋₁₀ aryl; and

X is C₃₋₂₀ alkylene.

In some embodiments:

R¹ is H or C₁₋₆ alkyl;

R² is H or C₁₋₆ alkyl; and

X is C₄₋₁₀ alkylene.

In some embodiments, the at least one repeating unit has the structure selected from:

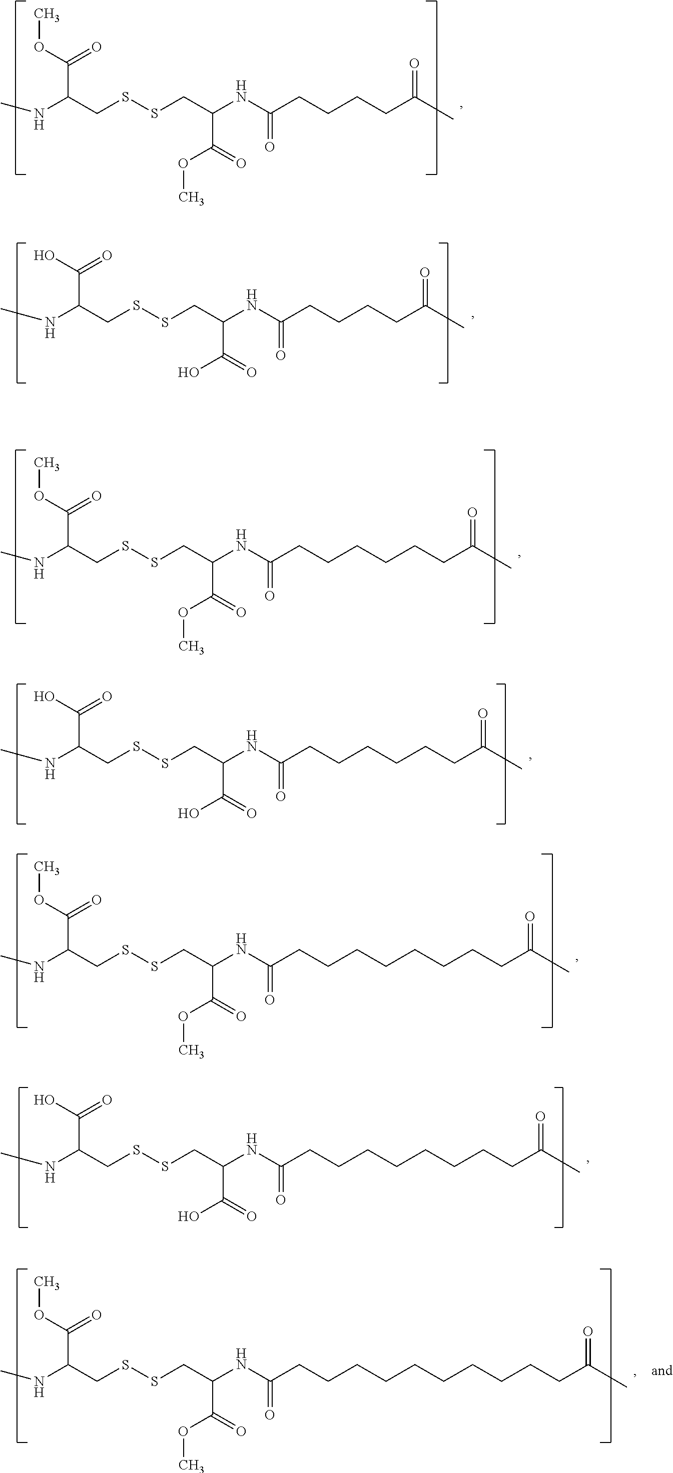

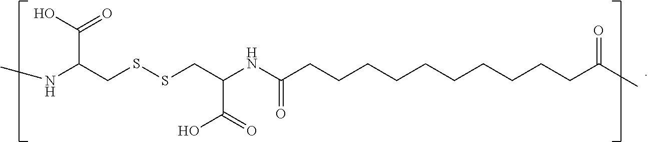

In some embodiments, the complexing agent is a cationic lipid or a cationic lipid-like material such as lipophilic moiety-modified amino dendrimer.

Suitable examples of lipophilic moieties with which an amino dendrimer may be modified include fatty acids and glycerides. Examples of fatty acids include saturated and unsaturated fatty acids, such as linolenic acid, linoleic acid, myristic acid, stearic acid, palmitic acid, eicosanoic acid, and margaric acid. Examples of fatty glycerides include 1,2-dipalmitoyl-sn-glycero-3-phosphoethanolamine, 1,2-dioleoyl-sn-glycero-3-phosphoethanolamine, 1,2-dimyristoyl-sn-glycero-3-phosphoethanolamine, and 1,2-distearoyl-sn-glycero-3-phosphoethanolamine.

In some embodiments, the cationic lipid is selected from 1,2-dioleoyl-3-trimethylammonium-propane (DOTAP) and 1,2-di-O-octadecenyl-3-trimethylammonium propane (DOTMA); and the lipophilic moiety-modified amino dendrimer is selected from polypropylenimine tetramine dendrimer generation 1 modified with a lipophilic moiety, ethylenediamine core-poly (amidoamine) (PAMAM) generation 0 dendrimer (G0) modified with C14 (G0-C14 dendrimer); and ethylenediamine branched polyethyleneimine modified with lipophilic moiety.

In some embodiments, the weight ratio of the complexing agent to the p53-encoding mRNA in the core of the particle is from about 5 to about 20.

In some embodiments, the amphiphilic material comprises one or more compounds selected from neutral, cationic and anionic lipids, PEG-phospholipid, and a PEG-ceramide.

In some embodiments, the amphiphilic material comprises 1,2-dimyristoyl-sn-glycero-3-phosphoethanolamine-N-[methoxy(polyethylene glycol)] (DMPE-PEG) or 1,2-distearoyl-sn-glycero-3-phosphoethanolamine-N-[methoxy(polyethylene glycol)] (DSPE-PEG), or a combination thereof.

In some embodiments, the mTOR inhibitor is everolimus, or a pharmaceutically acceptable salt thereof. In some embodiments, the platinum-based antineoplastic agent is cisplatin, or a pharmaceutically acceptable salt thereof. In some embodiments, the AMPK activating agent is metformin, or a pharmaceutically acceptable salt thereof.

In some embodiments, the cancer is selected from lung cancer and liver cancer.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIGS. 1A-D. In vitro transfection efficiency of the redox-responsive mRNA NPs in p53-null Hep3B cells. (A) Transmission electron microscopy (TEM) images of the hybrid mRNA NPs before incubation (in PBS) or after incubation in 10 mM DTT for 2 or 4 hours at 37° C. (B) Confocal laser scanning microscopy (CLSM) images of p53-null Hep3B cells after incubation with naked Cy5-labeled mRNA (red) for 6 hours, and with engineered Cy5-labeled mRNA NPs for 1, 3, or 6 hours. Endosomes were stained by Lysotracker Green (green) and nuclei were stained by DAPI (blue). Scale bars, 50 μm. (C) In vitro transfection efficiency (% EGFP positive cells) was determined by flow cytometry. Data shown as means±S.E.M. (n=3), and statistical significance was determined using two-tailed t test (**P<0.01). (D) Histogram analysis of the in vitro transfection efficiency by Flowjo software.

FIGS. 2A-I. Restoration of p53 functions in p53-null Hep3B cells by the mRNA NPs and in vitro mechanisms for p53 restoration-mediated anti-tumor effect. (A) Immunofluorescence (IF) staining of p53 in the p53-null Hep3B cells treated by empty NP or p53-mRNANPs (scale bars, 50 μm). (B) The viability of the p53-null Hep3B liver cancer cells after treatment with PBS, empty NPs, naked p53-mRNA (0.830 μg/ml), or p53-mRNA NPs (mRNA concentration: 0.103, 0.207, 0.415, or 0.830 μg/ml) by AlarmBlue assay. Statistical significance was determined using two-tailed t test (*P<0.05, **P<0.01). (C) Colony formation assays of Hep3B cells after treatment with empty NPs vs. p53-mRNA NPs in 6-well plates. (D) Apoptosis of Hep3B cells as determined by flow cytometry after treatment with empty NPs, naked p53-mRNA, or p53-mRNANPs. (E) Histogram analysis of the cell apoptosis (%) by Flowjo software. Data shown as means±S.E.M. (n=3), and statistical significance was determined using two-tailed t test (***P<0.001). (F) Cell cycle distributions of Hep3B cells after treatment with PBS, empty NPs, naked p53-mRNA, or p53-mRNA NPs (mRNA concentration: 0.830 μg/ml). (G) Western blot (WB) analysis of cell cycle-related protein expression (p21 and CyclinEl) after treatment with p53-mRNA NPs (mRNA concentration: 0.830 μg/ml). GAPDH was used as the loading control. (H) WB analysis of mitochondrial apoptotic signaling pathway in p53-null Hep3B cells treated with PBS, empty NPs, naked p53-mRNA, or p53-mRNANPs (mRNA concentration: 0.830 μg/ml). BCL-2, BAX, PUMA, C-CAS9, and C-CAS3 proteins were detected. Actin was used as the loading control. (I) TEM images of the mitochondria morphology in Hep3B cells from control, empty NPs, and p53-mRNANPs groups (mRNA concentration: 0.830 μg/ml; blue arrow: normal mitochondria; red arrow: swelling mitochondria). Scale bars, 2 μm for the top images and 1 μm for the enlarged images (bottom).

FIGS. 3A-J. Mechanisms of the p53-mRNA NP-mediated sensitization to everolimus in p53-null Hep3B cells. (A) The viability of Hep3B cells after treatment with everolimus, as measured by AlamarBlue assay. Data shown as means±S.E.M. (n=3). (B) WB analysis of total mTOR, p-mTOR, and p-p70S6K after treatment with everolimus at different concentrations. Actin was measured as the loading control. (C) WB analysis of p-mTOR, LC3B-1, and LC3B-2. Actin was measured as the loading control. (D) TEM images of Hep3B cells before and after 24 hours of treatment with everolimus (32 nM). Autophagosomes were labelled by yellow arrows (scale bars from left to right: 2, 5, and 1 μm). (E) CLSM images of GFP-LC3-transfected Hep3B cells from different treatment groups (scale bars, 50 μm). (F) WB analysis of p53, p-mTOR, total m-TOR, p-4EBP1, BECN1, LC3B-1/LC3B-2, BCL-2, BAX, C-CAS9, and C-CAS3 in Hep3B cells after different treatments. Actin was used as the loading control. (G) Left: TEM images of Hep3B cells in control, p53-mRNA NPs, everolimus, and p53-mRNA NPs+everolimus groups (mRNA concentration: 0.415 μg/ml; everolimus concentration: 32 nM). Scale bars, 2 μm for the raw images and 1 μm for the enlarged images. Yellow arrows: autophagosomes; Red arrows: mitochondria. Right: Statistical analysis of the numbers of autophagosomes (yellow) and swollen mitochondria (red) after different treatments. (H) The viability of Hep3B cells in different groups (control, EGFP-mRNA NPs, p53-mRNA NPs, everolimus, or p53-mRNA NPs+everolimus), as measured by AlamarBlue assay (mRNA concentration: 0.415 μg/ml; everolimus concentration: 32 nM). Data shown as means±S.E.M. (n=3), and statistical significance was determined using two-tailed t test (**P<0.01, ***P<0.001). (I) Colony formation of Hep3B cells in different treatment groups in 6-well plates. (J) Flow cytometry analysis of the cell apoptosis (AnnV+PI− and AnnV+PI+). The percentage of apoptotic Hep3B cells was shown in the histogram. Statistical significance was determined using two-tailed t test (***P<0.001).

FIGS. 4A-K. Anti-tumor effects of p53-mRNA NPs are synergistic with everolimus in p53-null HCC xenograft model. (A) Blood circulation profiles of naked Cy5-labeled mRNA and Cy5-labeled mRNA NPs (at an mRNA dose of 750 μg per kg of animal weight). NP₂₅, NP₅₀, and NP₇₅ represent three different ratios of DSPE-PEG/DMPE-PEG (25:75, 50:50, and 75:25) hybrid in the lipid-PEG layer of hybrid NPs. Data shown as means S.E.M. (n=3). (B) Time-lapse NIR fluorescence imaging of nude mice bearing p53-null HCC xenograft tumors after intravenous injection of free Cy5-mRNA, Cy5-mRNA NP₂₅, Cy5-mRNA NP₅₀, or Cy5-mRNA NP₇₅. The tumors were annotated with white arrows. (C) Scheme of tumor inoculation (s.c.) and treatment schedule in Hep3B tumor-bearing athymic nude mice. Twelve days after tumor inoculation, mice were treated with PBS (IV), EGFP-mRNA NPs (IV), p53-mRNA NPs (IV), everolimus (oral), or p53-mRNA NPs (IV)+everolimus (oral) every three days for 6 rounds (mRNA dose: 750 μg/kg; everolimus dose: 5 mg/kg). Tumors from different groups were harvested eighteen days after the last treatment. (D) Photos of excised tumors from mice bearing Hep3B xenografts in different treatment groups on Day 33 (n=5). (E-I) Individual tumor growth kinetics in (E) control, (F) EGFP-mRNA NPs, (G) everolimus, (H) p53-mRNA NPs, and (I) p53-mRNA NPs+everolimus group (n=5). (J) Average tumor growth kinetics for all treatment groups. Data shown as means±S.E.M. (n=5), and significance was determined using two-tailed t test (***P<0.001). (K) Average tumor volumes at experimental endpoint (Day 33) in all groups. Data shown as means±S.E.M. (n=5), and statistical significance was determined using two-tailed t test (***P<0.001). (L) IF images of p53 (red) and C-CAS3 (green) co-stained Hep3B tumor sections at 12, 24, 48, and 60 hours after IV injection of p53-mRNANPs. PBS (60 hours after IV injection) was used as control group (scale bars, 100 μm).

FIGS. 5A-C. In vivo mechanisms underlying the p53-mRNA NP-mediated sensitization of p53-null HCC xenograft model to everolimus. (A) Immunohistochemistry (IHC) images from tumor sections of Hep3B tumor-bearing xenograft mice before and after treatment with p53-mRNANPs (mRNA dose: 750 μg/kg). The protein expressions of p53, apoptotic markers (BAX and C-CAS3), and proliferation markers (Ki67 and PCNA) were evaluated by IHC staining (blue: nucleus; brown: p53, BAX, C-CAS3, Ki67, or PCNA). Scale bars, 100 μm. (B) CLSM images of fixed tumor tissues with the TUNEL staining (blue: nucleus; red: apoptosis) from PBS, EGFP-mRNA NPs, p53-mRNA NPs, everolimus, and p53-mRNANPs+everolimus groups (scale bars, 100 μm). (C) WB analysis of p53, LC3B-1, LC3B-2, BECN1, p62, BCL-2, BAX, C-CAS9, C-CAS3, and p-4EBP1 in the Hep3B xenograft tumors after different treatments. Actin was used as the loading control.

FIGS. 6A-G. Therapeutic efficacy in the p53-null orthotopic HCC tumors and the liver metastases of p53-null NSCLC. (A) Scheme of tumor inoculation and different treatments in luciferase-expressing Hep3B (Hep3B-Luc) orthotopic tumor-bearing nude mice. Twenty-one days after tumor inoculation, mice were treated with PBS (IV), EGFP-mRNA NPs (IV), p53-mRNANPs (IV), everolimus (oral), or p53-mRNANPs (IV)+everolimus (oral) every three days for 4 rounds (mRNA dose: 750 μg/kg; everolimus dose: 5 mg/kg). (B) Bioluminescence images of the Hep3B-Luc orthotopic tumor-bearing nude mice at Day 0, 6, and 12. (C) Average radiance of tumor burden determined by bioluminescence imaging at different time points. (D) Average radiance of tumor burden at the endpoint (Day 12). Data shown as means±S.E.M. (n=3), and statistical significance was determined using two-tailed t test (*P<0.05, **P<0.01). (E) Scheme of tumor inoculation and different treatments in p53-null H1299 metastatic tumor-bearing nude mice. Twenty-eight days after tumor inoculation, mice were treated with PBS (IV), EGFP-mRNANPs (IV), p53-mRNANPs (IV), everolimus (oral), or p53-mRNANPs (IV)+everolimus (oral) every three days for 5 rounds (mRNA dose: 750 μg/kg; everolimus dose: 5 mg/kg). Organs from different groups were harvested three days after the final treatment. (F) Histological examination of liver tissues from each group by H&E staining. The metastatic lesions (red dotted ovals) were identified as cell clusters with darkly stained nuclei (scale bars, 100 μm). (G) The number of metastatic nodules in the liver from each group. One liver was randomly selected from each group with a blind method, and the liver section from each group was divided into four regions for counting of the metastasis nodules. Data shown as means±S.E.M. (n=4 regions), and statistical significance was determined using two-tailed t test (*P<0.05, **P<0.01).

FIGS. 7A-B. Study summary. (A) Schematic representation of the synthesis of chemically modified mRNA and the formulation of redox-responsive lipid-polymer hybrid NPs for mRNA delivery. After intravenous injection, the synthetic mRNA NPs enter tumor tissues through the enhanced permeability and retention (EPR) effect for targeting tumor cells, followed by (1) NP endocytosis; (2) endosomal escape; and (3) redox-responsive release of (4) mRNA from the NPs. The released mRNA can then induce restoration of tumor suppressor proteins such as p53. (B) Schematic representation of the mechanism of p53-mRNANP-mediated sensitization of cells to everolimus by inhibiting the activation of protective autophagy inp53-deficient cancer cells. Along with p53 restoration-induced apoptosis and cell cycle arrest, the combination of p53-mRNA NPs with everolimus is expected to show synergistic anti-tumor effect.

FIG. 8. The structure schematic of synthetic mRNA. It includes an anti-reverse cap analog (ARCA), untranslated regions (UTRs), an open reading frame (ORF), and a poly-A tail.

FIG. 9. The chemical structure of 3′-O-Me-m⁷G(5′)ppp(5′)G ARCA cap.

FIGS. 10A-B. Chemicals for NP synthesis. (A) Chemical structures of the lipid-PEGs (DMPE-PEG and DSPE-PEG), polymer (PDSA), and cationic lipid-like material (G0-C14). (B) ¹H NMR spectrum of the synthesized redox-responsive polymer PDSA.

FIGS. 11A-C. Characterization of the engineered hybrid mRNANPs. (A) Agarose gel electrophoresis assay of mRNA in nuclease-free water, DMF, or complexed with cationic G0-C14 at various weight ratios. The engineered mRNA NPs were also subjected to gel electrophoresis for detecting any mRNA leaching. (B) Stability of the engineered mRNA NPs over 3 days in PBS containing 10% serum at 37° C. (C) In vitro release of Cy5-labeled mRNA from the engineered NPs in PBS, 1 mM DTT, and 10 mM DTT at 37° C. Data shown as means±S.E.M. (n=3).

FIG. 12. Size of EGFP-mRNA NPs and Luc-mRNA NPs with various formulations. NP formulations with different ratios of composition are listed in table S1. Data shown as means±S.E.M. (n=3).

FIG. 13. Encapsulation efficiency of EGFP-mRNA NPs and Luc-mRNA NPs with various formulations. NP formulations with different ratios of composition are listed in table S1. Data shown as means±S.E.M. (n=3).

FIG. 14. Normalized luminescence intensity of Hep3B cells after treatment with various Luc-mRNANP formulations at the mRNA dose of 0.830 μg/ml. NP formulations with different ratios of composition are listed in table S1. Data shown as means±S.E.M. (n=3).

FIGS. 15A-D. Endosomal escape of mRNANPs. Confocal laser scanning microscopy (CLSM) images of p53-null H1299 NSCLC cells after incubation with (A) naked Cy5-labeled mRNA (red) for 6 h, and (B-D) Cy5-labeled mRNA NPs for (B) 1 h, (C) 3 h, and (D) 6 h. Endosomes were stained by Lysotracker Green (green) and nuclei were stained by DAPI (blue). Scale bar, 50 μm.

FIGS. 16A-F. Transfection efficacy verified by CLSM imaging. CLSM images of p53-null Hep3B cells transfected with (A) naked EGFP-mRNA, (B) EGFP-mRNA NPs, and (C) EGFP-mRNA Lip2k; and p53-null H1299 cells transfected with (D) naked EGFP mRNA, (E) EGFP-mRNA NPs, and (F) EGFP-mRNA Lip2k (scale bar, 100 μm).

FIGS. 17A-I. Transfection efficacy verified by flow cytometry. Histogram analysis of the in vitro transfection efficiency in the p53-null H1299 NSCLC cells treated with (A) PBS, (B) empty NPs, (C) naked EGFP-mRNA (0.830 μg/ml), (D) EGFP-mRNA NPs (0.103 μg/ml), (E) EGFP-mRNA NPs (0.207 μg/ml), (F) EGFP-mRNA NPs (0.415 μg/ml), (G) EGFP-mRNANPs (0.830 μg/ml), and (H) EGFP-mRNA Lip2k (0.830 μg/ml) by Flowjo software. (I) In vitro transfection efficiency (% EGFP positive cells) was determined by flow cytometry. Data shown as means±S.E.M. (n=3), and statistical significance was determined using two-tailed t test (**P<0.01).

FIGS. 18A-F. Transfection efficacy after quenching intracellular GSH. Histogram analysis of the in vitro transfection efficiency in the p53-null Hep3B cells treated with (A) Nem (50 μM), (B) EGFP-mRNANPs (0.415 μg/ml), (C) Nem (50 μM) for 1 h followed by the EGFP-mRNANPs (0.415 μg/ml), (D) EGFP-mRNANPs (0.830 μg/ml), and (E) Nem (50 μM) for 1 h followed by the EGFP-mRNA NPs (0.830 μg/ml). (F) In vitro transfection efficiency (% EGFP positive cells) was determined by flow cytometry. Data shown as means±S.E.M. (n=3), and statistical significance was determined using two-tailed t test (***P<0.001).

FIGS. 19A-B. In vitro toxicity of the synthetic EGFP-mRNANPs. The viability of the (A) p53-null Hep3B cells and (B) p53-null H1299 cells after treatment with PBS, empty NPs, naked EGFP-mRNA (0.830 μg/ml), EGFP-mRNANPs (0.103, 0.207, 0.415, or 0.830 μg/ml), or EGFP-mRNA Lip2k (0.830 μg/ml), as measured by AlamarBlue assay.

FIGS. 20A-B. IF staining of p53 inp53-null H1299 cells. Cells were treated with (A) empty NPs or (B) p53-mRNA NPs (scale bars, 25 μm).

FIG. 21. WB analysis of p53 protein expression. Both p53-null Hep3B cells and p53-null H1299 cells were treated with PBS, empty NPs, naked p53-mRNA, or p53-mRNANPs. Actin was measured as the loading control.

FIGS. 22A-B. In vitro therapeutic efficacy of the synthetic p53-mRNANPs in p53-null H1299 cells. (A) The viability of H1299 cells after treatment with PBS, empty NPs, naked p53-mRNA (0.830 μg/ml), or p53-mRNANPs (0.103, 0.207, 0.415, or 0.830 μg/ml), as measured by AlamarBlue assay. Statistical significance was determined by two-tailed t test (***P<0.001). (B) Colony formation of H1299 cells after treatment with empty NPs vs. p53-mRNANPs in 6-well plates.

FIGS. 23A-F. Apoptosis of p53-null H1299 cells as determined by flow cytometry after different treatments. Cells were treated with (A) PBS, (B) empty NPs, (C) naked p53-mRNA (0.830 μg/ml), (D) p53-mRNANPs (0.415 μg/ml), and (E) p53-mRNANPs (0.830 μg/ml). (F) Histogram analysis of apoptosis in the respective groups by Flowjo software. Data shown as means±S.E.M. (n=3), and statistical significance was determined using two-tailed t test (*P<0.05, **P<0.01).

FIGS. 24A-E. G1-phase cell cycle arrest induced by p53-mRNA NPs. (A) Cell cycle distributions of the p53-null H1299 cells after treatment with PBS, empty NPs, naked p53-mRNA, or p53-mRNANPs. (B-D) Analysis of cell percentages in each cell cycle phase after treatment with (B) PBS, (C) empty NPs, (D) naked p53-mRNA, and (E) p53-mRNA NPs.

FIG. 25. WB analysis of apoptotic signaling pathway in p53-null H1299 cells after different treatments. Cells were treated with PBS, empty NPs, naked p53-mRNA, or p53-mRNANPs. p53, BCL-2, BAX, PUMA, cleaved caspase9 (C-CAS9), and cleaved caspase3 (C-CAS3) proteins were detected. Actin was used as the loading control.

FIG. 26. TEM images of mitochondria morphology inp53-null H1299 cells after different treatments. Images were obtained from control, empty NPs, and p53-mRNA NPs groups (blue arrow: normal mitochondria; red arrow: swelling mitochondria; scale bars in the raw images: 2 μm; scale bars in the enlarged images: 1 μm).

FIGS. 27A-C. In vitro toxicity of the mutant p53-R175H-mRNANPs. (A) WB analysis of p53, p21 (cell cycle-related protein), and C-CAS3 (apoptotic marker) protein expression in both p53-null Hep3B cells and p53-null H1299 cells after treatment with p53-R175H-mRNANPs. Actin was measured as the loading control. (B) p53-null Hep3B cells and (C) p53-null H1299 cells after treatment with PBS, empty NPs, or p53-R175H-mRNA NPs (0.830 μg/ml), as measured by AlamarBlue assay.

FIGS. 28A-B. Cytotoxicity of everolimus inp53-null H1299 cells. (A) Viability of H1299 cells after treatment with everolimus, as measured by AlamarBlue assay. Data shown as means±S.E.M. (n=3). (B) WB analysis of total mTOR, p-mTOR, and p-p70S6K after treatment with everolimus at different concentrations. Actin was used as the loading control.

FIGS. 29A-C. Effect of everolimus on autophagy activation inp53-null H1299 cells. (A) WB analysis of p-mTOR, LC3B-1, and LC3B-2 after treatment with everolimus in H1299 cells. Actin was used as the loading control. (B) TEM images of H1299 cells before and after treatment with everolimus. Increased number of autophagosomes (green arrows) could be visualized after 24 h treatment of everolimus (scale bars from left to right: 10 μm, 2 μm, and 1 μm). (C) CLSM images of p53-null H1299 cells transfected with GFP-LC3B from different groups (scale bars, 50 μm). Everolimus induced autophagosomes (green), whereas co-treatment with everolimus and p53-mRNA NPs inhibited everolimus-induced autophagy (reduced green fluorescence).

FIG. 30. WB analysis of autophagy and apoptotic signaling pathways in p53-null H1299 cells. p53, p-mTOR, total mTOR, BECN1, LC3B-1, LC3B-2, BCL-2, C-CAS9, and C-CAS3 in H1299 cells were assessed after different treatments. Actin was used as the loading control.

FIGS. 31A-B. Analysis of the autophagosomes and swollen mitochondria inp53-null H1299 cells after different treatments. (A) TEM images of the H1299 cells in control, p53-mRNANPs, everolimus, and p53-mRNANPs+everolimus groups (n=3; numbers represent different batches of test). An increased number of autophagosomes (yellow arrows) could be observed after treatment with everolimus, whereas changes to mitochondria morphology (red arrows) were also seen after treatment with p53-mRNA NPs (scale bars, 2 μm for the raw images and 1 μm for the enlarged images). (B) Statistical analysis of the numbers of autophagosomes (yellow) and swollen mitochondria (red) after different treatments in (A).

FIGS. 32A-B. In vitro therapeutic efficacy of the combination of p53-mRNA NPs with everolimus inp53-null H1299 cells. (A) Viability of H1299 cells in different groups (control, EGFP-mRNANPs, p53-mRNANPs, everolimus, or p53-mRNANPs+everolimus), as measured by AlamarBlue assay. The concentration of mRNA used was 0.415 μg/ml, and the concentration of everolimus was 16 nM. Data shown as means±S.E.M. (n=3), and statistical significance was determined using two-tailed t test (**P<0.01, ***P<0.001). (B) Colony formation of H1299 cells after different treatments in 6-well plate.

FIGS. 33A-F. In vitro apoptosis of p53-null H1299 cells after different treatments. Flow cytometry analysis of cell apoptosis (AnnV+PI- and AnnV+PI+) after treatment with (A) PBS, (B) EGFP-mRNA NPs, (C) p53-mRNA NPs, (D) everolimus, or (E) p53-mRNA NPs+everolimus. (F) Histogram of the percentage of apoptotic H1299 cells from (A-E). Data shown as means±S.E.M. (n=3), and statistical significance was determined using two-tailed t test (***P<0.001).

FIGS. 34A-B. In vitro toxicity of the combination of everolimus with venetoclax. Cell viability of (A) p53-null Hep3B cells and (B) p53-null H1299 cells after treatment with everolimus (Hep3B, E1: 8 nM, E2: 16 nM, and E3: 32 nM; H1299, E1: 4 nM, E2: 8 nM, and E3: 16 nM), venetoclax (N4: 40 nM, N5: 80 nM, and N6:160 nM), or the combination of both drugs, as measured by AlamarBlue assay. Data shown as means±S.E.M. (n=3).

FIGS. 35A-C. In vitro toxicity of the combination of everolimus with siBcl-2. (A) Cell viability of p53-null Hep3B cells after treatment with PBS, lipofectamine 2000 (Lip2k), Lip2k/siBcl-2 (10 nM), everolimus (8, 16, or 32 nM), or the combination of Lip2k/siBcl-2 with everolimus, as measured by AlamarBlue assay. (B) Cell viability of p53-null H1299 cells after treatment with PBS, Lip2k, Lip2k/siBcl-2 (10 nM), Everolimus (4, 8, or 16 nM), or the combination of Lip2k/siBcl-2 with everolimus, as measured by AlamarBlue assay. Data shown as means±S.E.M. (n=6). (C) WB analysis of the expression of BCL-2 in Hep3B and H1299 cells after Lip2k/siBcl-2 treatments. Actin was used as the loading control.

FIGS. 36A-B. The relative mRNA expression of p53. Cells were treated with p53-mRNA NPs, everolimus, or p53-mRNANPs+everolimus. The relative mRNA expression of p53 in (A) Hep3B and (B) H1299 cells was analyzed after 24 h treatment. Cells without any treatment were used as the control.

FIGS. 37A-B. The relative mRNA expression of ULK1, ATG7, BECN1, and ATG12. (A) Hep3B cells and (B) H1299 cells were analyzed after 24 h of treatment with p53-mRNA NPs, everolimus, or p53-mRNA NPs+everolimus. Cells without any treatment were used as control group.

FIGS. 38A-B. The relative mRNA expression of DRAM1, ISG20L1, and SESN1. (A) Hep3B cells and (B) H1299 cells were analyzed after 24 h of treatment with p53-mRNA NPs, everolimus, or p53-mRNA NPs+everolimus. Cells without any treatment were used as control group.

FIGS. 39A-B. The relative mRNA expression of TIGAR. (A) Hep3B and (B) H1299 cells were analyzed after 24 h treatment with p53-mRNA NPs, everolimus, or p53-mRNA NPs+everolimus. Cells without any treatment were used as the control.

FIG. 40. WB analysis of AMPK and TIGAR pathways. p53, p-AMPKα, p-ACCα, TIGAR, BECN1, LC3B-1, and LC3B-2 in Hep3B cells (left) and H1299 cells (right) were assessed after different treatments. Actin was used as the loading control.

FIG. 41. Schematic representation of the possible mechanism by which p53 tumor suppressor inhibits protective autophagy and sensitizes tumor cells to everolimus.

FIGS. 42A-B. Biodistribution of different mRNA NPs in HCC xenograft tumor model. (A) Biodistribution of naked Cy5-labeled mRNA and Cy5-labeled mRNA NPs in different organs (H: heart Li: liver, S: spleen, Lu: lungs, and K: kidneys) and Hep3B tumors. NP₂₅, NP₅₀, and NP₇₅ represent three different ratios of DSPE-PEG/DMPE-PEG in the lipid-PEG layer of hybrid mRNA NPs. (B) Quantification of biodistribution of naked Cy5-labeled mRNA and Cy5-labeled mRNA NPs from (A). Data shown as means±S.E.M. (n=3).

FIGS. 43A-B. Biodistribution of different mRNA NPs in NSCLC xenograft tumor model. (A) Biodistribution of naked Cy5-labeled mRNA and Cy5-labeled mRNA NPs in different organs (H: heart, Li: liver, S: spleen, Lu: lungs, and K: kidneys) and H1299 tumors. NP₂₅, NP₅₀, and NP₇₅ represent three different ratios of DSPE-PEG/DMPE-PEG in the lipid-PEG layer of hybrid mRNA NPs. (B) Quantification of biodistribution of naked Cy5-labeled mRNA and Cy5-labeled mRNA NPs from (A). Data shown as means±S.E.M. (n=3).

FIG. 44. Blood vessel staining in tumor sections. CLSM images of the tumor sections from the p53-null HCC xenograft model and p53-null NSCLC xenograft model (scale bar, 400 μm). The nuclei of tumor cells were stained by DAPI (blue), and the blood vessels were stained by anti-CD31 (green).

FIGS. 45A-B. Efficacy and safety of different treatments in HCC xenograft model. (A) Whole-body images of mice bearing p53-null Hep3B xenograft tumors treated with PBS, EGFP-mRNANPs, everolimus, p53-mRNA NPs, or p53-mRNANPs+everolimus (Day 35). (B) Average body weight of Hep3B tumor-bearing mice over the course of therapy. Data shown as means±S.E.M. (n=5).

FIGS. 46A-I. Anti-tumor effects of p53-mRNANPs are synergistic with everolimus in NSCLC xenograft model. (A) Scheme of tumor inoculation (s.c.) and treatment schedule in H1299 tumor-bearing athymic nude mice. Fourteen days after tumor inoculation, mice were treated with PBS (IV), EGFP-mRNA NPs (IV), p53-mRNA NPs (IV), everolimus (oral), or p53-mRNA NPs (IV)+everolimus (oral) every three days for 6 rounds (mRNA dose: 750 μg/kg; everolimus dose: 5 mg/kg). Tumors from different groups were harvested three days after the final treatment. (B) Photos of excised tumors from mice bearing H1299 xenografts in different treatment groups on Day 18 (n=5). (C) Average tumor growth kinetics for all treatment groups. Data shown as means±S.E.M. (n=5), and significance was determined using two-tailed t test (***P<0.001). (D) Average tumor volumes at the experimental endpoint (Day 18) in all groups. Data shown as means±S.E.M. (n=5), and statistical significance was determined using two-tailed t test (***P<0.001). (E-I) Individual tumor growth kinetics in the (E) control, (F) EGFP-mRNA NPs, (G) everolimus, (H) p53-mRNA NPs, and (I) p53-mRNANPs+everolimus groups (n=5). Insets: Representative mouse photographs at the experimental endpoint (Day 18). The arrows indicate the tumors on mice.

FIGS. 47A-B. Murine p53 restoration in p53-null murine liver cancer RIL-175 cells. (A) WB analysis of the expression of mouse p53 protein after treatment with murine p53-mRNANPs. Actin was used as the loading control. (B) Viability of p53-null murine liver cancer cell RIL-175 after treatment with empty NPs or murine p53-mRNA NPs (0.830 μg/ml), as measured by AlamarBlue assay. Data shown as means±S.E.M. (n=4), and statistical significance was determined using two-tailed t test (***P<0.001).

FIGS. 48A-G. Therapeutic efficacy of murine p53-mRNA NPs in immunocompetent mice bearing p53-null RIL-175 tumors. (A) Scheme of tumor inoculation (s.c.) and treatment schedule in RIL-175 tumor-bearing C57BL/6 mice. Ten days after tumor inoculation, mice were treated with PBS (IV), EGFP-mRNA NPs (IV), or murine p53-mRNA NPs (IV) every three days for 6 rounds (at an mRNA dose of 750 μg per kg of animal weight). (B) Whole-body images of immunocompetent mice bearing p53-null RIL-175 liver tumors treated with PBS, EGFP-mRNA NPs, or murine p53-mRNA NPs (Day 18). (C-E) Individual tumor growth kinetics in the (C) control, (D) EGFP-mRNA NPs, and (E) murine p53-mRNA NPs groups (n=5). (F) Average tumor growth kinetics for all treatment groups. Data shown as means±S.E.M. (n=5), and significance was determined using two-tailed t test (**P<0.01). (G) Average tumor volumes at the experimental endpoint (Day 18) in all groups. Data shown as means±S.E.M. (n=3), and statistical significance was determined using two-tailed t test (**P<0.01).

FIG. 49. Expression of p53 protein in HCC xenograft model after treatment with p53-mRNANPs. IF images of p53 (red) and nucleus (blue) co-stained in Hep3B tumor sections at 12 h after IV injection of p53-mRNA NPs. Empty NPs were used as control group (scale bars, 300 μm).

FIG. 50. Expression of p53 protein in NSCLC xenograft model after treatment with p53-mRNA NPs. IF images of p53 (red) and nucleus (blue) co-stained in H1299 tumor sections at 12 h post IV injection of p53-mRNANPs. Empty NPs was used as control group (scale bars, 300 μm).

FIG. 51. IHC images from tumor sections of H1299 tumor-bearing mice before and after treatment with p53-mRNA NPs. The protein expressions of p53, TIGAR, LC3B, Ki67, and C-CAS3 were evaluated by IHC staining (blue: nucleus; brown: p53, TIGAR, LC3B, Ki67, or C-CAS3; scale bars, 100 μm).

FIGS. 52A-B. In vivo toxicity of the p53-mRNA NP-mediated strategy for everolimus rescue assessed by histopathological and hematological analysis. (A) H&E staining of sections of the major organs (heart, liver, spleen, lung, and kidney) was performed three days after the last administration of PBS, EGFP-mRNANPs, everolimus, p53-mRNANPs, or p53-mRNA NPs+everolimus (scale bars, 100 μm). (B) Analysis of serum biochemistry and whole blood parameters: alanine aminotransferase (ALT), aspartate aminotransferase (AST), urea nitrogen (BUN), red blood cells (RBC), white blood cells (WBC), hemoglobin (Hb), mean corpuscular hemoglobin concentration (MCHC), mean corpuscular hemoglobin (MCH), hematocrit (HCT), and lymphocyte count (LY).

FIG. 53. IHC images from major organs and tumor sections of the HCC xenograft model. The protein expressions of p53 and apoptotic marker (C-cas3) were evaluated by IHC staining (blue: nucleus; brown: p53 or C-cas3) with or without the treatment of p53-mRNA NPs (scale bars, 100 μm).

FIGS. 54A-D. Evaluation of immune responses after treatment with mRNA NPs. Serum concentrations of (A) IFN-γ, (B) TNF-α, (C) IL-12, and (D) IL-6 at 24 h after injection of PBS, empty NPs, or p53-mRNA NPs in immunocompetent BALB/c mice.

FIGS. 55A-E. Scans of the liver metastases from different treatment groups in FIG. 6. The five groups include (A) PBS control, (B) EGFP-mRNANPs, (C) Everolimus, (D) p53-mRNA NPs, and (E) p53-mRNA NPs+Everolimus.

FIG. 56. Table summarizing compositions of different NP formulations FIGS. 57A-B. Table summarizing different p53-mRNA sequences used the present application (A—Human p53-mRNA Open Reading Frame (ORF) sequence, Mutant human p53-R175H-mRNA ORF sequence, B—Murine p53-mRNA ORF sequence).

FIG. 58. Table summarizing primer sequences for qRT-PCR.

FIG. 59 Cell viability of A549, H1299, and H1975 after different treatments: control NPs, p53 mRNANPs, cisplatin, and cisplatin with p53 mRNANPs. Cis-1 and Cis-2 represent cisplatin treatment with two different concentrations.

FIG. 60 Cell viability of A549, H1299, and H1975 after different treatments: control NPs, p53 mRNA NPs, metformin, and metformin with p53 mRNA NPs. Met-1 and Met-2 represent cisplatin treatment with two different concentrations.

### DETAILED DESCRIPTION

The mammalian target of rapamycin (mTOR) is a serine/threonine kinase that regulates major cell functions such as growth and proliferation in physiological and pathological conditions (1). Dysregulation of the mTOR signaling pathway has been reported for a wide range of cancers including liver and lung cancers (2-4). Everolimus (RAD001) is an effective mTOR inhibitor that has been clinically approved for several types of cancers, such as advanced kidney cancer and pancreatic neuroendocrine tumor. However, everolimus failed to improve survival in patients with other advanced cancers, such as hepatocellular carcinoma (HCC) or non-small cell lung cancer (NSCLC) (5-8). Previous studies have proposed several mechanisms underlying the variable response or resistance to everolimus in different tumor cells (9, 10), including the activation of pro-survival autophagy (11-13) and the dysregulation of apoptotic pathways (for example, upregulation of anti-apoptotic protein BCL-2) (14). Combining everolimus with autophagy or BCL-2 inhibitors improved anti-tumor efficacy, but these inhibitors could also induce undesired toxicities by interfering with physiological processes in normal cells (15-17).

In parallel to the gain of pro-tumorigenic functions such as the mTOR signaling pathway, cancer is also frequently associated with the inactivation of tumor suppressors. p53 is one of the most widely altered tumor suppressor genes in numerous cancers. For example, the loss of p53 function has been widely detected in ˜36% of HCC and ˜68% of NSCLC, according to The Cancer Genome Atlas (TCGA) database in the cBio Cancer Genomics Portal (18). p53 regulates many important cellular pathways. As a transcription factor, p53 can activate its downstream genes in response to oncogenic signals (19), such as pro-apoptotic proteins BAX (BCL-2 associated X protein) and PUMA (p52 up-regulated modulator of apoptosis) (20). p53 also acts as a cell cycle checkpoint guard to induce cell cycle arrest (21) and participates in DNA replication and repair to protect genomic integrity (22). In addition, cytoplasmic (but not nuclear) p53 inhibits the activation of protective autophagy that may contribute to the tolerance to chemotherapies (23, 24). Therefore, the restoration of p53 expression could potentially not only inhibit tumor growth by inducing cell apoptosis and cell cycle arrest, but also sensitize p53-deficient cancers to the mTOR inhibitor (e.g., everolimus) and other anti-cancer agents, such as AMPK activators and DNA alkylating agents.

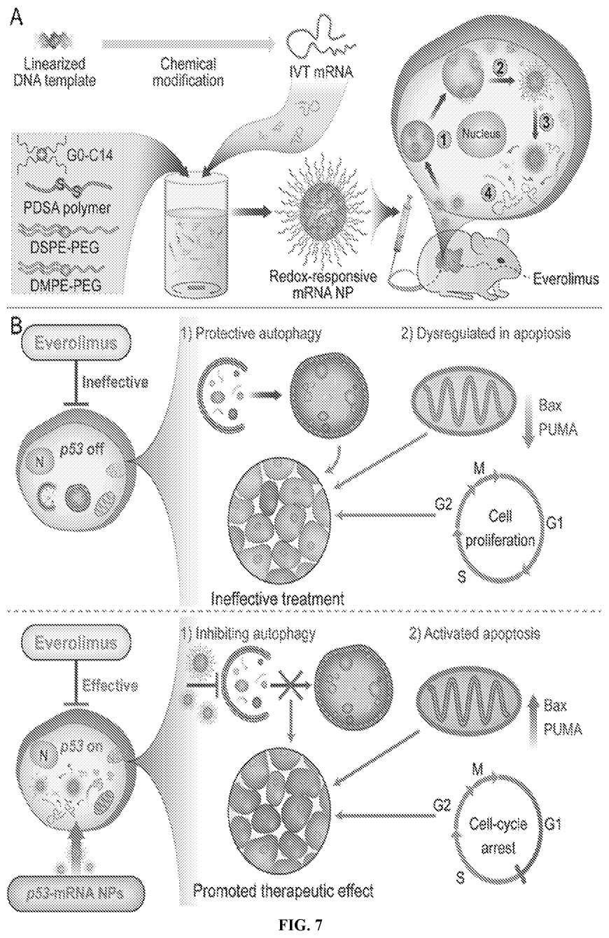

Two different strategies have been widely explored for p53 reactivation: i) the use of small molecules to disrupt the p53-MDM2 (mouse double minute 2 homolog) interaction and release p53 or to restore wild-type function to mutant p53 by covalent modification of its core domain (25-28), and ii) the restoration of a functional copy via viral or non-viral DNA transfection (29-31). Although these attempts have exhibited some successes, each has formidable limitations. For instance, small-molecular compounds are likely ineffective when the tumor suppressor gene has been deleted, and p53-DNA-based gene therapies have the potential risk of genomic integration and mutagenesis (32, 33). The present application provides a method of use of messenger RNA (mRNA) to reconstitute p53 expression inp53-deficient HCC and NSCLC with redox-responsive lipid-polymer hybrid nanoparticles (NPs) engineered for effective delivery of synthetic mRNA (FIG. 7A). Because mRNA functions in the cytoplasm, this strategy advantageously avoids the requirement of nuclear localization and the risk of insertional mutagenesis associated with DNA (34, 35). The experimental results presented herein demonstrate that treatment of p53-null Hep3B HCC and H1299 NSCLC cells with the p53-mRNA hybrid NPs inhibited tumor cell growth by inducing cell apoptosis and G1-phase cell cycle arrest. The p53-mRNA NPs also sensitized these tumor cells to everolimus, e.g., via p53 restoration-mediated regulation of the autophagy pathway (FIG. 7B), resulting in synergistic anti-tumor efficacy in vitro and in vivo.

Methods of Treating

The compounds, particles, combinations, and methods of the present disclosure may be used to treat a pathology, disease, or condition in a subject (e.g., a subject in need thereof). The subject may be in need of treatment when diagnosed with the disease, pathology, or condition by a competent physician (e.g., oncologist).

In some embodiments, the disease or condition is cancer. Suitable examples of cancer include bladder cancer, brain cancer, breast cancer, colorectal cancer (e.g., colon cancer), rectal cancer, cervical cancer, gastrointestinal cancer, genitourinary cancer, head and neck cancer, lung cancer, oral cancer, ovarian cancer, pancreatic cancer (e.g., pancreatic neuroendocrine tumor), prostate cancer, endometrial cancer, renal cancer (kidney cancer) (e.g., advanced kidney cancer), skin cancer, liver cancer, thyroid cancer, leukemia, and testicular cancer.

In some embodiments, cancer is selected from sarcoma, angiosarcoma, fibrosarcoma, rhabdomyosarcoma, liposarcoma, myxoma, rhabdomyoma, fibroma, lipoma, teratoma, lung cancer, non-small cell lung cancer (NSCLC), bronchogenic carcinoma squamous cell, undifferentiated small cell, undifferentiated large cell, adenocarcinoma, alveolar bronchiolar carcinoma, bronchial adenoma, sarcoma, chondromatous hamartoma, mesothelioma, gastrointestinal cancer, cancer of the esophagus, squamous cell carcinoma, adenocarcinoma, leiomyosarcoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma, hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, brain cancer, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

In some embodiments, the cancer is p53-deficient or has a mutant p53 gene (e.g., having a mutation that mutes a p53 function). Main p53 functions consist of cell cycle arrest, DNA repair, senescence, and apoptosis induction. Hence, the cancer that is p53-deficient or has a mutant p53 gene lack these cellular functions. In one example, the p53-deficient cancer or cancer that has a p53-mutated gene does not undergo apoptotic cell death and continue to proliferate, despite, e.g., serious DNA damaging events. In some embodiments, the method of treating a patient includes a step of determining that the cancer contains a mutation or an alteration in the p53 gene or that the cancer is p53-deficient (the cancer is lacking at least one molecular function associated with p53 gene). In one example, this step can be carried out without obtaining a cancer cell from a subject. For example, a p53 mutation or deficiency can be identified by analyzing blood sample of the subject, or a sample of hair, urine, saliva, or feces of the subject for an appropriate biomarker. In some embodiments, a p53 mutation or deficiency can be identified by obtaining a cancer cell from a subject. For example, a cancer cell for analysis of a p53 mutation can be obtained from the subject by surgical means (e.g., laparoscopically), by image-guided biopsy, using a fine needle aspiration (FNA), a surgical tissue harvesting, a punch biopsy, a liquid biopsy, a brushing, a swab, or a touch-prep.

Any of the methods, reagents, protocols and devices generally known in the art can be used to identify a p53 mutation or deficiency. For example, next generation sequencing, immunohistochemistry, fluorescence microscopy, break apart FISH analysis, Southern blotting, Western blotting, FACS analysis, Northern blotting, ELISA or ELISPOT, antibodies microarrays, or immunohistochemistry, and PCR-based amplification (e.g., RT-PCR and quantitative real-time RT-PCR) techniques can be used to identify the mutation or a POLQ status of cancer. As is well-known in the art, the assays are typically performed, e.g., with at least one labelled nucleic acid probe or at least one labelled antibody or antigen-binding fragment thereof. Assays can utilize other detection methods known in the art for detecting a mutation in a p53-associated gene. Any DNA sequencing platform for somatic mutations can be used. For example, Illumina MiSeq platform (Illumina TruSeq Amplicon Cancer Hotspot panel, 47 gene), or NextSeq (Agilent SureSelect XT, 592 gene selected based on COSMIC database) can be used to identify a p53 mutation or deficiency. The sample can be a biological sample or a biopsy sample (e.g., a paraffin-embedded biopsy sample) from the patient. In some embodiments, the patient is a patient suspected of having a cancer having a mutation or deficiency in a p53-associated gene.

Active Ingredients

mRNA Encoding p53 Protein

The present methods include delivering mRNA encoding a tumor suppressor p53 to a cell (e.g., a cancer cell). Exemplary sequences of the p53 mRNA are shown in FIG. 57. However, multiple transcript variants and mutants can be used in the methods of the present disclosure. The methods can include using an mRNA sequence for the variant that is predominantly expressed in a normal, non-cancerous cell of the same type as the tumor. The methods can include using a nucleotide sequence coding for an mRNA that is at least 80% identical to a reference sequence in FIG. 57. The methods can include using a nucleotide sequence coding for an mRNA that is at least 80% identical to a reference sequence in Table A below

TABLE A

| | Genetic | Associated | GenBank Acc No. | |
|---|---|---|---|---|
| GENE | Alteration(s) | Cancer(s) | mRNA | Protein |
| p53 | Point | Lung | AF307851.1 | AAG28785.1 |
| | mutation, | Prostate | NM_000546.5 | NP_000537.3 |
| | deletion | | | |

In some embodiments, the nucleotide sequences are at least 85%, 90%, 95%, 99% or 100% identical to those described in FIG. 57 or Table A. To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100%) of the length of the reference sequence. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

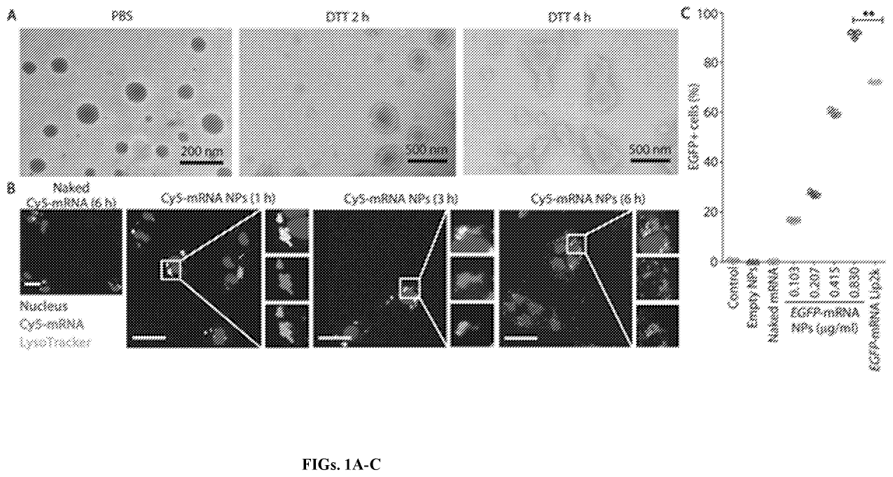

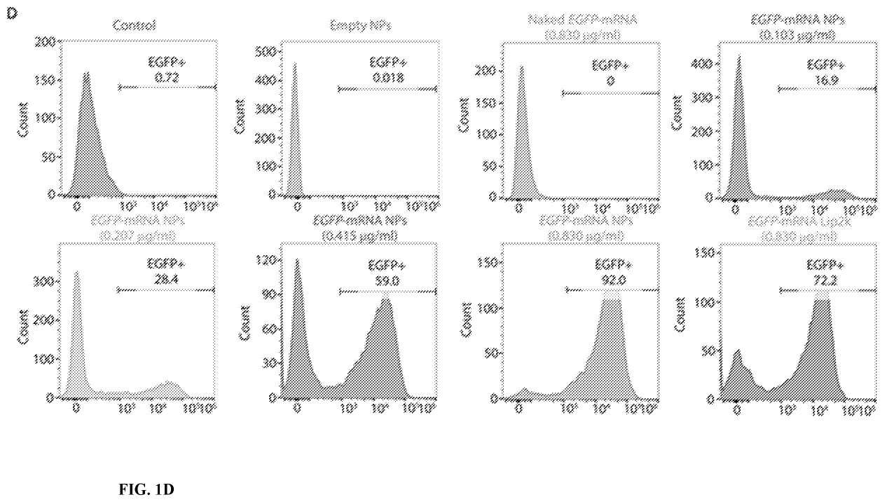

A mature mRNA is generally comprised of five distinct portions (see FIG. 1a of Islam et al., Biomater Sci. 2015 December; 3(12):1519-33): (i) a cap structure, (ii) a 5′ untranslated region (5′ UTR), (iii) an open reading frame (ORF), (iv) a 3′ untranslated region (3′ UTR) and (v) a poly(A) tail (a tail of 100-250 adenosine residues). Typically, the mRNA will be in vitro transcribed using methods known in the art. The mRNA will typically be modified, e.g., to extend half-life or to reduce immunogenicity. For example, the mRNA can be capped with an anti-reverse cap analog (ARCA), in which OCH₃ is used to replace or remove natural 3′ OH cap groups to avoid inappropriate cap orientation. Tetraphosphate ARCAs or phosphorothioate ARCAs can also be used (Islam et al. 2015). The mRNA is preferably enzymatically polyadenylated (addition of a poly adenine (A) tail to the 3′ end of mRNA), e.g., to comprise a poly-A tail of at least 100 or 150 As. Typically poly(A) polymerase is used; E. coli poly(A) polymerase (E-PAP) I has been optimized to add a poly(A) tail of at least 150 adenines to the 3′ terminal of in vitro transcribed mRNA. Preferably, any adenylate-uridylate rice elements (AREs) are removed or replaced with 3′ UTR of a stable mRNA species such as β-globin mRNA. Iron responsive elements (IREs) can be added in the 5′ or 3′ UTR. In some embodiments, the mRNAs include full or partial (e.g., at least 50%, 60%, 70%, 80%, or 90%) substitution of cytidine triphosphate and uridine triphosphate with naturally occurring 5-methylcytidine and pseudouridine (ψ) triphosphate. See Islam et al., 2015, and references cited therein.

mTOR Inhibitors

In some embodiments, the methods within the present claims include administering to a patient an inhibitor of mammalian target of rapamycin (mTOR). mTOR is the catalytic subunit of two structurally distinct complexes: mTORC1 and mTORC2. mTOR Complex 1 (mTORC1) is composed of mTOR, regulatory-associated protein of mTOR (Raptor), mammalian lethal with SEC13 protein 8 (mLST8) and the non-core components PRAS40 and DEPTOR. This complex functions as a nutrient, energy, and redox sensor and controls protein synthesis. mTOR Complex 2 (mTORC2) is composed of MTOR, rapamycin-insensitive companion of MTOR (RICTOR), MLST8, and mammalian stress-activated protein kinase interacting protein 1 (mSIN1). mTORC2 has been shown to function as an important regulator of the actin cytoskeleton through its stimulation of F-actin stress fibers, paxillin, RhoA, Rac1, Cdc42, and protein kinase C α (PKCα). mTORC2 also phosphorylates the serine/threonine protein kinase Akt/PKB on serine residue Ser473, thus affecting metabolism and survival. Phosphorylation of Akt's serine residue Ser473 by mTORC2 stimulates Akt phosphorylation on threonine residue Thr308 by PDK1 and leads to full Akt activation. In addition, mTORC2 exhibits tyrosine protein kinase activity and phosphorylates the insulin-like growth factor 1 receptor (IGF-IR) and insulin receptor (InsR) on the tyrosine residues Tyr1131/1136 and Tyr1146/1151, respectively, leading to full activation of IGF-IR and InsR. In some embodiments, the mTOR inhibitor within the present claims inhibits mTOR1 (e.g., any of the subunits of mTOR1). In some embodiments, the mTOR inhibitor within the present claims inhibits mTOR2 (e.g., any of the subunits of mTOR2).

Suitable examples of mTOR inhibitors include rapamycin, everolimus, sirolimus, temsirolimus, ridaforolimus, deforolimus, dactolisib, BGT226, SF1126, PKI-587, NVPBE235, sapanisertib, AZD8055, AZD2014, XL765, and OSI027, or a pharmaceutically acceptable salt thereof.

Platinum-Based Antineoplastic Agents

Platinum-based antineoplastic agents typically are coordination complexes of platinum (II or IV). Platinum-based antineoplastic agents cause crosslinking of DNA. Mostly they act on the adjacent N-7 position of guanine, forming a 1,2 intrastrand crosslink. The resultant crosslinking inhibits DNA repair and/or DNA synthesis in a cancer cell, and causes the death of the cancer cell. The platinum-based antineoplastic agents are commonly used to treat testicular cancer, ovarian cancer, cervical cancer, breast cancer, bladder cancer, head and neck cancer, esophageal cancer, lung cancer, mesothelioma, brain tumors and neuroblastoma, and are usually administered to the subject by an injection. Suitable examples of platinum-based antineoplastic agents include cisplatin, oxaliplatin, carboplatin, nedaplatin, triplatin tridentate, phenanthriplatin, picoplatin, eptaplatin, dicycloplatin, miriplatin, and satraplatin, or a pharmaceutically acceptable salt thereof.

AMPK Activating Agent

5′ AMP-activated protein kinase (AMPK) is typically activated by biguanide drugs (metformin and phenformin). This enzyme plays a role in cellular energy homeostasis, typically to activate glucose and fatty acid uptake and oxidation when cellular energy is low. It consists of three proteins (subunits) that together make a functional enzyme. In response to binding AMP and ADP, the net effect of AMPK activation is stimulation of hepatic fatty acid oxidation, ketogenesis, stimulation of skeletal muscle fatty acid oxidation and glucose uptake, inhibition of cholesterol synthesis, lipogenesis, and triglyceride synthesis, inhibition of adipocyte lipogenesis, and activation of adipocyte lipolysis. Activated AMPK adjusts its downstream channels through the cascade (e.g. acetyl-CoA carboxylase (ACC), mechanistic target of rapamycin (mTOR), tuberous sclerosis 1/2 (TSC1/2) to induce the cancer cell death by producing material and energy situation. In some embodiments, the AMPK activating agent is a direct AMPK activator. In other embodiments, the AMPK activating agent is an indirect AMPK activator. Suitable examples of AMPK activating agents include metformin, phenformin, 2-Deoxy-D-glucose (2DG), 5-aminoimidazole-4-carboxamide ribonucleotide (AICAR), resveratrol, biguanides, curcumin, salicylate, A-769662, Compound 991, MT 63-78, PT-1, OSU-53, Compound-13, and CNX-012-570, or a pharmaceutically acceptable salt thereof. The AMPK activator may be any one of the AMPK activator compounds described in Chen et al., Oncotarget, 2017 8, 56, 96089-96102, which is incorporated herein by reference in its entirety.

mRNA Delivery Vehicles

In some embodiments of the present methods and compositions, the mRNA encoding a tumor suppressor is within a delivery vehicle. The delivery vehicle can include, inter alia, protamine complexes and particles such as lipid nanoparticles, polymeric nanoparticles, lipid-polymer hybrid nanoparticles, and inorganic (e.g., gold) nanoparticles, e.g., as described in Islam et al., 2015.

Particles may be microparticles or nanoparticles. Nanoparticles are preferred for intertissue application, penetration of cells, and certain routes of administration. The nanoparticles may have any desired size for the intended use. The nanoparticles may have any diameter from 10 nm to 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In preferred embodiments the nanoparticles can have a diameter less than 400 nm, less than 300 nm, or less than 200 nm. The preferred range is between 50 nm and 300 nm.

Nanoparticles can be polymeric particles, non-polymeric particles (e.g., a metal particle, quantum dot, ceramic, inorganic material, bone, etc.), liposomes, micelles, polymeric micelles, viral particles, hybrids thereof, and/or combinations thereof. In some embodiments, the nanoparticles are, but not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. In some embodiments, nanoparticles can comprise one or more polymers or co-polymers.

Nanoparticles may be a variety of different shapes, including but not limited to spheroidal, cubic, pyramidal, oblong, cylindrical, toroidal, and the like. Nanoparticles can comprise one or more surfaces.

In some embodiments, the nanoparticles present within a population, e.g., in a composition, can have substantially the same shape and/or size (i.e., they are “monodisperse”). For example, the particles can have a distribution such that no more than about 5% or about 10% of the nanoparticles have a diameter greater than about 10% greater than the average diameter of the particles, and in some cases, such that no more than about 8%, about 5%, about 3%, about 1%, about 0.3%, about 0.1%, about 0.03%, or about 0.01% have a diameter greater than about 10% greater than the average diameter of the nanoparticles.

In some embodiments, the diameter of no more than 25% of the nanoparticles varies from the mean nanoparticle diameter by more than 150%, 100%, 75%, 50%, 25%, 20%, 10%, or 5% of the mean nanoparticle diameter. It is often desirable to produce a population of nanoparticles that is relatively uniform in terms of size, shape, and/or composition so that most of the nanoparticles have similar properties. For example, at least 80%, at least 90%, or at least 95% of the nanoparticles produced using the methods described herein can have a diameter or greatest dimension that falls within 5%, 10%, or 20% of the average diameter or greatest dimension. In some embodiments, a population of nanoparticles can be heterogeneous with respect to size, shape, and/or composition. In this regard, see, e.g., WO 2007/150030, which is incorporated herein by reference in its entirety.

Liposomes

In some embodiments, nanoparticles may optionally comprise one or more lipids. In some embodiments, a nanoparticle may comprise a liposome. In some embodiments, a nanoparticle may comprise a lipid bilayer. In some embodiments, a nanoparticle may comprise a lipid monolayer. In some embodiments, a nanoparticle may comprise a micelle.

In these delivery vehicles, the p53 mRNA is in the hollow core of the liposome or the micelle.

Hybrid Particles

In some embodiments, the delivery vehicle is a particle (e.g., a nanoparticle) comprising a water-insoluble polymeric core.

The water-insoluble polymeric core can comprise a variety of materials. The water-insoluble polymer can comprise homopolymers (i.e., synthesized from hydrophobic monomers (e.g., styrene, methyl methacrylate, glycidyl methacrylate, DL-lactide, and the like)), random copolymers (i.e., synthesized from two or more monomers (e.g., styrene, methyl methacrylate, glycidyl methacrylate, DL-lactide, acrylic acid, methacrylic acid, 2-hydroxyethyl acrylate, and the like)), block polymers (i.e., synthesized from two or more monomers (e.g., styrene, methyl methacrylate, glycidyl methacrylate, DL-lactide, acrylic acid, methacrylic acid, 2-hydroxyethyl acrylate, and the like)), graft polymers (e.g., synthesized from artificial polymers (polyacrylic acid, polyglycidyl methacrylate, and the like) and/or natural polymers (e.g., dextran, starch, chitosan, and the like) with functional pendent groups (e.g., amino, carboxylate, hydroxyl, epoxy groups, and the like)), and/or branched polymers (e.g., a hyperbranched polyester with multifunctional alcohol building block and 2,2-bis(methylol)propionic acid branching units, such as Boltorn™ H40).

Non-limiting exemplary polymers that can be included in the polymeric core include polymer systems that are approved for use in humans, e.g., poly(glycolic acid), poly(lactic acid), poly(caprolactone), poly(lactide-co-glycolide), poly(ortho ester) II, poly(alkyl cyanoacrylate), desaminotyrosyl octyl ester, polyphosphoesters, polyester amides, polyurethanes, and lipids. Other non-limiting examples of polymers that the core can comprise include: chitosan; acrylates copolymer; acrylic acid-isooctyl acrylate copolymer; ammonio methacrylate copolymer; ammonio methacrylate copolymer type A; ammonio methacrylate copolymer type B; butyl ester of vinyl methyl ether/maleic anhydride copolymer (125,000 molecular weight); carbomer homopolymer type A (allyl pentaerythritol crosslinked); carbomer homopolymer type B (allyl sucrose crosslinked); cellulosic polymers; dimethylaminoethyl methacrylate-butyl methacrylate-methyl methacrylate copolymer; dimethylsiloxane/methylvinylsiloxane copolymer; divinylbenzene styrene copolymer; ethyl acrylate-methacrylic acid copolymer; ethyl acrylate and methyl methacrylate copolymer (2:1; 750,000 molecular weight); ethylene vinyl acetate copolymer; ethylene-propylene copolymer; ethylene-vinyl acetate copolymer (28% vinyl acetate); glycerin polymer solution i-137; glycerin polymer solution im-137; hydrogel polymer; ink/polyethylene terephthalate/aluminum/polyethylene/sodium polymethacrylate/ethylene vinyl acetate copolymer; isooctyl acrylate/acrylamide/vinyl acetate copolymer; Kollidon® VA 64 polymer; methacrylic acid-ethyl acrylate copolymer (1:1) type A; methacrylic acid-methyl methacrylate copolymer (1:1); methacrylic acid-methyl methacrylate copolymer (1:2); methacrylic acid copolymer; methacrylic acid copolymer type A; methacrylic acid copolymer type B; methacrylic acid copolymer type C; octadecene-1/maleic acid copolymer; PEG-22 methyl ether/dodecyl glycol copolymer; PEG-45/dodecyl glycol copolymer; Polyester polyamine copolymer; poly(ethylene glycol) 1,000; poly(ethylene glycol) 1,450; poly(ethylene glycol) 1,500; poly(ethylene glycol) 1,540; poly(ethylene glycol) 200; poly(ethylene glycol) 20,000; poly(ethylene glycol) 200,000; poly(ethylene glycol) 2,000,000; poly(ethylene glycol) 300; poly(ethylene glycol) 300-1,600; poly(ethylene glycol) 300-1,600; poly(ethylene glycol) 3,350; poly(ethylene glycol) 3,500; poly(ethylene glycol) 400; poly(ethylene glycol) 4,000; poly(ethylene glycol) 4,500; poly(ethylene glycol) 540; poly(ethylene glycol) 600; poly(ethylene glycol) 6,000; poly(ethylene glycol) 7,000; poly(ethylene glycol) 7,000,000; poly(ethylene glycol) 800; poly(ethylene glycol) 8,000; poly(ethylene glycol) 900; polyvinyl chloride-polyvinyl acetate copolymer; povidone acrylate copolymer; povidone/eicosene copolymer; polyoxy(methyl-1,2-ethanediyl), alpha-hydro-omega-hydroxy-, polymer with 1,1′-methylenebis[4-isocyanatocyclohexane] copolymer; polyvinyl methyl ether/maleic acid copolymer; styrene/isoprene/styrene block copolymer; vinyl acetate-crotonic acid copolymer; {poly[(9,9-di-n-octylfluorenyl-2,7-diyl)-alt-(benzo[2,1,3]thiadiazol-4,8-diyl)]}, and {poly[2,6-(4,4-bis-(2-ethylhexyl)-4H-cyclopenta [2,1-b;3,4-b′]dithiophene)-alt-4,7(2,1,3-benzothiadiazole)]}.

In some embodiments, the water-insoluble core comprises a hydrophobic polymer. Non-limiting examples of hydrophobic polymers include, but are not limited to: polylactic acid (PLA), polypropylene oxide, poly(lactide-co-glycolide) (PLGA), poly(epsilon-caprolactone), poly(ethylethylene), polybutadiene, polyglycolide, polymethylacrylate, polyvinylbutylether, polystyrene, polycyclopentadienyl-methylnorbornene, polyethylenepropylene, polyethylethylene, polyisobutylene, polysiloxane, a polymer of any of the following: methyl acrylate, ethyl acrylate, propyl acrylate, n-butyl acrylate, isobutyl acrylate, 2-ethyl acrylate, t-butyl acrylate, methacrylates (e.g., ethyl methacrylate, n-butyl methacrylate, and isobutyl methacrylate), acrylonitriles, methacrylonitrile, vinyls (e.g., vinyl acetate, vinylversatate, vinylpropionate, vinylformamide, vinylacetamide, vinylpyridines, and vinyllimidazole), aminoalkyls (e.g., aminoalkylacrylates, aminoalkylsmethacrylates, aminoalkyl(meth)acrylamides), styrenes, and lactic acids.

In some embodiments, the water-insoluble core comprises an amphipathic polymer. Amphipathic polymers contain a molecular structure containing one or more repeating units (monomers) connected by covalent bonds and the overall structure includes both hydrophilic (polar) and lipophilic (apolar) properties, e.g., at opposite ends of the molecule. In some embodiments, the amphipathic polymers are copolymers containing a first hydrophilic polymer and a first hydrophobic polymer. Several methods are known in the art for identifying an amphipathic polymer. For example, an amphipathic polymer (e.g., an amphipathic copolymer) can be identified by its ability to form micelles in an aqueous solvent and/or Langmuir Blodgett films.

In some embodiments, the amphipathic polymer (e.g., an amphipathic copolymer) contains a polymer selected from the group of: polyethylene glycol (PEG), polyethylene oxide, polyethyleneimine, diethyleneglycol, triethyleneglycol, polyalkylene glycol, polyalkyline oxide, polyvinyl alcohol, polyvinylpyrrolidone, polyvinylmethylether, polymethyloxazoline, polyethyloxazoline, polyhydroxypropyl-oxazoline, polyhydroxypropylmethacrylamide, polymethacrylamide, polydimethylacryl-amide, polyhydroxypropylmethacrylate, polyhydroxyethylacrylate, hydroxymethylcellulose, hydroxyethylcellulose, polyglycerine, polyaspartamide, polyoxyethlene-polyoxypropylene copolymer (poloxamer), a polymer of any of lecithin or carboxylic acids (e.g., acrylic acid, methacrylic acid, itaconic acid, and maleic acid), polyoxyethylenes, polyethyleneoxide, and unsaturated ethylenic monocarboxylic acids. In some embodiments, the amphipathic polymer contains a polymer selected from the group of: polylactic acid (PLA), polypropylene oxide, poly(lactide-co-glycolide) (PLGA), poly(epsilon-caprolactone), poly(ethylethylene), polybutadiene, polyglycolide, polymethylacrylate, polyvinylbutylether, polystyrene, polycyclopentadienylmethylnorbornene, polyethylenepropylene, polyethylethylene, polyisobutylene, polysiloxane, and a polymer of any of the following: methyl acrylate, ethyl acrylate, propyl acrylate, n-butyl acrylate, isobutyl acrylate, 2-ethyl acrylate, t-butyl acrylate, methacrylates (e.g., ethyl methacrylate, n-butyl methacrylate, and isobutyl methacrylate), acrylonitriles, methacrylonitrile, vinyls (e.g., vinyl acetate, vinylversatate, vinylpropionate, vinylformamide, vinylacetamide, vinylpyridines, and vinyllimidazole), aminoalkyls (e.g., aminoalkylacrylates, aminoalkylsmethacrylates, and aminoalkyl(meth)acrylamides), styrenes, and lactic acids.

In some embodiments, the amphipathic polymer contains poly(ethylene glycol)-co-poly(D,L-lactic acid) (PLA-PEG), poly(ethylene glycol)-co-(poly(lactide-co-glycolide)) (PLGA-PEG) (e.g., the amphipathic polymer is PLGA-PEG), polystyrene-b-polyethylene oxide, polybutylacrylate-b-polyacrylic acid, or polybutylmethacrylate-b-polyethyleneoxide. Additional examples of amphipathic copolymers are described in U.S. Patent Application Publication No. 2004/0091546 (incorporated herein by reference in its entirety). Additional examples of amphipathic polymers (e.g., amphipathic copolymers) are known in the art.

In some embodiments, the water-insoluble core comprises a polymer comprising an aliphatic polyester polymer, e.g., polycaprolactone (PCL), polybutylene succinate (PBS), or a polyhydroxylalkanoate (PHA), such as polyhydroxybutyrate. Other examples include polylactic acid (PLA) and polyglycolic acid (PGA). In some embodiments, the aliphatic polyester polymer is selected from polylactic acids, polyglycolic acids, and copolymers of lactic acid and glycolic acid (PLGA). A copolymer of lactic acid and glycolic acid can comprise a range of ratios of lactic acid to glycolic acid monomers, for example, from about 1:9 to about 9:1, from about 1:4 to about 4:1, from about 3:7 to about 7:3, or from about 3:2 to about 2:3. In some embodiments, the ratio of lactic acid to glycolic acid monomers can be about 1:9; about 1:8; about 1:7; about 1:6; about 1:5; about 1:4; about 3:7; about 2:3; about 1:1; about 3:2; about 7:3; about 4:1; about 5:1; about 6:1; about 7:1; about 8:1; or about 9:1.

In some embodiments, the water-insoluble core comprises a fluorescent polymer. The fluorescent polymer can be one or more polymers selected from polyphenylenevinylenes (e.g., poly[(2-methoxy-5-(2-ethylhexyloxy)-1,4-phenylene-vinylene)-co-(4,4′-biphenylene-vinylene)]), polyfluorenes (e.g., poly(fluorene-co-phenylene) (PFP), poly(9,9-dioctylfluorenyl-2,7-diyl); copolymers such as poly[{9,9-dioctyl-2,7-divinylene-fluorenylene}-alt-co-{2-methoxy-5-(2-ethylhexyloxy)-1,4-phenylene}]), polythiophenes (e.g., poly(3-butylthiophene-2,5-diyl), poly(3-decyl-thiophene-2,5-diyl), poly[3-(2-ethyl-isocyanato-octadecanyl)thiophene], poly(3,3′″-didodecyl quarter thiophene), copolymers such as poly[(9,9-dihexylfluorenyl-2,7-diyl)-alt-co-(bithiophene)] and poly[(9,9-dioctylfluorenyl-2,7-diyl)-alt-co-(bithiophene)]), poly(p-phenyleneethylene)s (PPE), polydiacetylenes (PDA), and their derivatives. Additional non-limiting examples of fluorescent polymers include F8BT {poly[(9,9-di-n-octylfluorenyl-2,7-diyl)-alt-(benzo[2,1,3]thiadiazol-4,8-diyl)]} and PCPDTBT {poly[2,6-(4,4-bis-(2-ethylhexyl)-4H-cyclopenta [2,1-b;3,4-b′]dithiophene)-alt-4,7(2,1,3-benzothiadiazole)]}.

In some embodiments, the water-insoluble polymeric core consists essentially of, or consists of, one or more polymers described herein.

In certain embodiments, the hydrophobic polymer is a polymer comprising at least one repeating unit according to Formula (I):

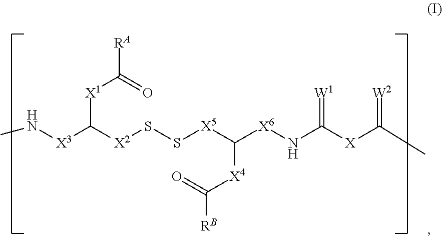

X¹ is a bond or C₁₋₁₀₀ alkylene;

X² is C₁₋₁₀₀ alkylene;

X³ is a bond or C₁₋₁₀₀ alkylene;

X⁴ is a bond or C₁₋₁₀₀ alkylene;

X⁵ is C₁₋₁₀₀ alkylene;

X⁶ is a bond or C₁₋₁₀₀ alkylene;

R^{A} is OR¹ or NR¹R⁴;

R^{B} is OR² or NR²R⁴;

R¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₁₀₀ alkyl or C(═O)R⁶;

each R⁴ is independently H or C₁₋₁₀₀ alkyl;

each R⁵ is independently H or C₁₋₁₀₀ alkyl;

each R⁶ is independently H or C₁₋₁₀₀ alkyl;

W¹ is O, S, or NH;

W² is O, S, or NH;

X is C₁₋₁₀₀ alkylene, C₂₋₁₀₀ alkenylene, or C₂₋₁₀₀ alkynylene;

provided that when W¹ and W² are both O, then X is C₃₋₁₀₀ alkylene, C₂₋₁₀₀ alkenylene, or C₂₋₁₀₀ alkynylene; and

each m is 0, 1 or 2.

In some embodiments, X¹ is a bond or C₁₋₄ alkylene.

In some embodiments, X² is C₁₋₄ alkylene.

In some embodiments, X³ is a bond or C₁₋₄ alkylene.

In some embodiments, X⁴ is a bond or C₁₋₄ alkylene.

In some embodiments, X⁵ is C₁₋₄ alkylene.

In some embodiments, X⁶ is a bond or C₁₋₄ alkylene.

In some embodiments, R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl.

In some embodiments, R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl.

In some embodiments, each R³ is independently H, C₁₋₆ alkyl or C(═O)R⁶.

In some embodiments, each R⁴ is independently H or C₁₋₆ alkyl.

In some embodiments, each R⁵ is independently H or C₁₋₆ alkyl.

In some embodiments, each R⁶ is independently H or C₁₋₆ alkyl.

In some embodiments, X is C₂₋₂₀ alkylene, C₂₋₂₀ alkenylene, or C₂₋₂₀ alkynylene.

In some embodiments,

X¹ is a bond or C₁₋₄ alkylene;

X² is C₁₋₄ alkylene;

X³ is a bond or C₁₋₄ alkylene;

X⁴ is a bond or C₁₋₄ alkylene;

X⁵ is C₁₋₄ alkylene;

X⁶ is a bond or C₁₋₄ alkylene;

R^{A} is OR¹ or NR³R⁴;

R^{B} is OR² or NR²R⁴;

R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₆ alkyl or C(═O)R⁶;

each R⁴ is independently H or C₁₋₆ alkyl;

each R⁵ is independently H or C₁₋₆ alkyl;

each R⁶ is independently H or C₁₋₆ alkyl;

W¹ is O, S, or NH;

W² is O, S, or NH;

X is C₂₋₂₀ alkylene, C₂₋₂₀ alkenylene, or C₂₋₂₀ alkynylene; and

each m is 0, 1 or 2.

In some embodiments, when W¹ is O and W² is O, X is C₃₋₂₀ alkylene, C₂₋₂₀ alkenylene, or C₂₋₂₀ alkynylene. For example, X can be C₃₋₂₀ alkylene.

In some embodiments, when W¹ is O and W² is O, X is C₄₋₂₀ alkylene, C₂₋₂₀ alkenylene, or C₂₋₂₀ alkynylene. For example, X can be C₄₋₂₀ alkylene.

In some embodiments, X¹ is a bond.

In some embodiments, X² is C₁₋₄ alkylene. For example, X² can be CH₂.

In some embodiments, X³ is a bond.

In some embodiments, X⁴ is a bond.

In some embodiments, X⁵ is C₁₋₄ alkylene. For example, X⁵ can be CH₂.

In some embodiments, X⁶ is a bond.

In some embodiments, R^{A} is OR¹.

In some embodiments, R^{B} is OR².

In some embodiments, W¹ is O.

In some embodiments, W² is O.

In some embodiments, a polymer of Formula (I) has at least one repeating unit with a structure according to Formula (Ia):

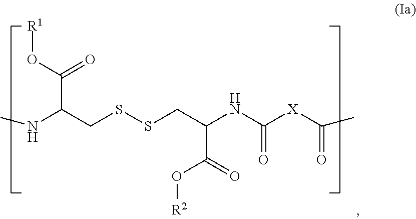

wherein:

R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₆ alkyl or C(═O)R⁶;

each R⁴ is independently H or C₁₋₆ alkyl;

each R⁵ is independently H or C₁₋₆ alkyl;

each R⁶ is independently H or C₁₋₆ alkyl;

X is C₃₋₂₀ alkylene, alkenylene, or alkynylene; and

each m is 0, 1 or 2.

In some embodiments, R¹ is H, C₁₋₂₀ alkyl, C₂₋₂₀ alkenyl, C₂₋₂₀ alkynyl, C₃₋₁₀ cycloalkyl, or C₆₋₁₀ aryl. For example, R¹ can be H. In some embodiments, R¹ is C₁₋₂₀ alkyl. In some embodiments, R¹ is C₁₋₆ alkyl. For example, R¹ can be CH₃. In some embodiments, R¹ is CH₂CH₃.

In some embodiments, R² is H, C₁₋₂₀ alkyl, C₂₋₂₀ alkenyl, C₂₋₂₀ alkynyl, C₃₋₁₀ cycloalkyl, or C₆₋₁₀ aryl. For example, R² can be H. In some embodiments, R² is C₁₋₂₀ alkyl. In some embodiments, R² is C₁₋₆ alkyl. For example, R² can be CH₃. In some embodiments, R² is CH₂CH₃.

In some embodiments, R³ is C₁₋₆ alkyl. For example, R³ can be CH₃. In some embodiments, R³ is H.

In some embodiments, R⁴ is C₁₋₆ alkyl. For example, R⁴ can be CH₃.

In some embodiments, R⁵ is C₁₋₆ alkyl. For example, R⁵ can be CH₃.

In some embodiments, R⁶ is C₁₋₆ alkyl. For example, R⁶ can be CH₃.

In some embodiments, m is 0. In some embodiments, m is 2.

The length and nature of the X group can be used to modulate the hydrophobicity of a polymer of Formula (I) and/or Formula (Ia). X groups may include alkylenes, including C₃₋₂₀ alkylenes (e.g, (CH₂)₃₋₂₀) and C₄₋₁₀ alkylenes (e.g, (CH₂)₄₋₁₀). Specific alkyl ene groups include C₄ alkylenes (e.g, (CH₂)₄), C₅ alkylenes (e.g, (CH₂)₅), C₆ alkylenes (e.g, (CH₂)₆), C₇ alkylenes (e.g, (CH₂)₇), C₈ alkylenes (e.g, (CH₂)₈), C₉ alkylenes (e.g, (CH₂)₉), C₁₀ alkylenes (e.g., (CH₂)₁₀), C₁₁ alkylenes (e.g., (CH₂)₁₁), and C₁₂ alkylenes (e.g., (CH₂)₁₂).

Examples of a repeating unit in a polymer of Formula (I) and/or Formula (Ia) where X is (CH₂)₄ include:

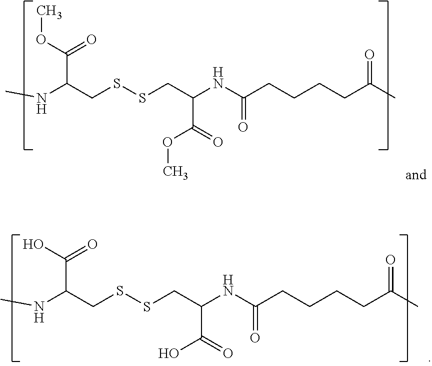

Examples of a repeating unit in a polymer of Formula (I) and/or Formula (Ia) where X is (CH₂)₆ include:

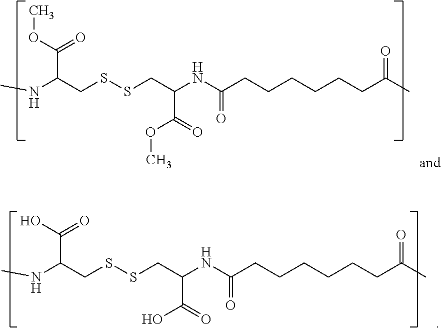

Examples of a repeating unit in a polymer of Formula (I) and/or Formula (Ia) where X is (CH₂)₈ include:

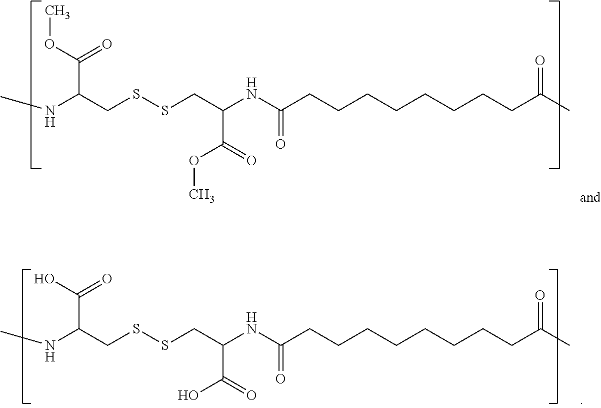

Examples of a repeating unit in a polymer of Formula (I) and/or Formula (Ia) where X is (CH₂)₁₀ include:

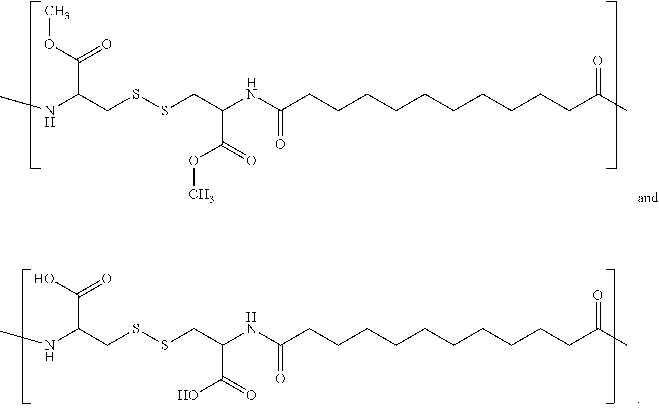

In some embodiments, the hydrophobic polymer comprises at least one repeating unit according to Formula (II):

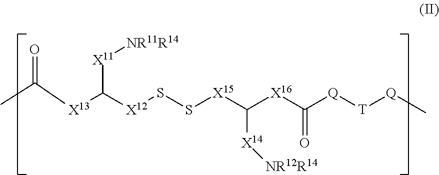

wherein:

X¹¹ is a bond or C₁₋₁₀₀ alkylene;

X¹² is C₁₋₁₀₀ alkylene;

X¹³ is a bond or C₁₋₁₀₀ alkylene;

X¹⁴ is a bond or C₁₋₁₀₀ alkylene;

X¹⁵ is C₁₋₁₀₀ alkylene;

X¹⁶ is a bond or C₁₋₁₀₀ alkylene;

R¹¹ is H, C₁₋₁₀o alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;

R¹² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;

each R¹³ is independently H, C₁₋₁₀₀ alkyl or C(═O)R¹⁶;

each R¹⁴ is independently H or C₁₋₁₀₀ alkyl;

each R¹⁵ is independently H or C₁₋₁₀₀ alkyl;

each R¹⁶ is independently H or C₁₋₁₀₀ alkyl;

each Q is independently O or NR¹⁷;

each R¹⁷ is H or C₁₋₁₀₀ alkyl;

T is C₂₋₁₀₀ alkylene, C₄₋₁₀₀ alkenylene, or C₄₋₁₀₀ alkynylene; and

each n is 0, 1 or 2.

In some embodiments, X¹¹ is a bond or C₁₋₄ alkylene.

In some embodiments, X¹² is C₁₋₄ alkylene.

In some embodiments, X¹³ is a bond or C₁₋₄ alkylene.

In some embodiments, X¹⁴ is a bond or C₁₋₄ alkylene.

In some embodiments, X¹⁵ is C₁₋₄ alkylene.

In some embodiments, X¹⁶ is a bond or C₁₋₄ alkylene.

In some embodiments, R¹¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl.

In some embodiments, R¹² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl.

In some embodiments, each R¹³ is independently H, C₁₋₆ alkyl or C(═O)R⁶.

In some embodiments, each R¹⁴ is independently H or C₁₋₆ alkyl.

In some embodiments, each R¹⁵ is independently H or C₁₋₆ alkyl.

In some embodiments, each R¹⁶ is independently H or C₁₋₆ alkyl.

In some embodiments, T is C₂₋₂₀ alkylene, C₂₋₂₀ alkenylene, or C₂₋₂₀ alkynylene.

In some embodiments,

X¹¹ is a bond or C₁₋₄ alkylene;

X¹² is C₁₋₄ alkylene;

X¹³ is a bond or C₁₋₄ alkylene;

X¹⁴ is a bond or C₁₋₄ alkylene;

X¹⁵ is C₁₋₄ alkylene;

X¹⁶ is a bond or C₁₋₄ alkylene;

R¹¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR³¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;

R¹² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;

each R¹³ is independently H, C₁₋₆ alkyl or C(═O)R¹⁶;

each R¹⁴ is independently H or C₁₋₆ alkyl;

each R¹⁵ is independently H or C₁₋₆ alkyl;

each R¹⁶ is independently H or C₁₋₆ alkyl;

each Q is independently O or NR¹⁷;

each R¹⁷ is independently H or C₁₋₆ alkyl;

T is C₂₋₂₀ alkylene, C₄₋₂₀ alkenylene, or C₄₋₂₀ alkynylene; and

each n is 0, 1 or 2.

In some embodiments, X¹¹ is a bond.

In some embodiments, X¹² is C₁₋₄ alkylene. For example, X¹² can be CH₂.

In some embodiments, X¹³ is a bond.

In some embodiments, X¹⁴ is a bond.

In some embodiments, X¹⁵ is C₁₋₄ alkylene. For example, X¹⁵ can be CH₂.

In some embodiments, X¹⁶ is a bond.

In some embodiments, R¹¹ is H, C₁₋₂₀ alkyl, C₂₋₂₀ alkenyl, C₂₋₂₀ alkynyl, C₃₋₁₀ cycloalkyl, or C₆₋₁₀ aryl. For example, R¹¹ can be H. In some embodiments, R¹¹ is C₁₋₂₀ alkyl. In some embodiments, R¹¹ is C₁₋₆ alkyl. For example, R¹¹ can be CH₃. In some embodiments, R¹¹ is CH₂CH₃.

In some embodiments, R¹² is H, C₁₋₂₀ alkyl, C₂₋₂₀ alkenyl, C₂₋₂₀ alkynyl, C₃₋₁₀ cycloalkyl, or C₆₋₁₀ aryl. For example, R¹² can be H. In some embodiments, R¹² is C₁₋₂₀ alkyl. In some embodiments, R¹² is C₁₋₆ alkyl. For example, R¹² can be CH₃. In some embodiments, R¹² is CH₂CH₃.

In some embodiments, R¹³ is C₁₋₆ alkyl. For example, R¹³ can be CH₃. In some embodiments, R¹³ is H.

In some embodiments, R¹⁴ is C₁₋₆ alkyl. For example, R¹⁴ can be CH₃.

In some embodiments, R¹⁵ is C₁₋₆ alkyl. For example, R¹⁵ can be CH₃.

In some embodiments, R¹⁶ is C₁₋₆ alkyl. For example, R¹⁶ can be CH₃.

In some embodiments, n is 0. In some embodiments, n is 2.

In some embodiments, Q is O.

The length and nature of the T group can be used to modulate the hydrophobicity of a polymer of Formula (II). T groups may include alkylenes, including C₃₋₂₀ alkylenes (e.g, (CH₂)₃₋₂₀) and C₄₋₁₀ alkylenes (e.g, (CH₂)₄₋₁₀). Specific alkylene groups include C₄ alkylenes (e.g., (CH₂)₄), C₅ alkylenes (e.g., (CH₂)₅), C₆ alkylenes (e.g., (CH₂)₆), C₇ alkylenes (e.g., (CH₂)₇), C₈ alkylenes (e.g, (CH₂)₈), C₉ alkylenes (e.g, (CH₂)₉), C₁₀ alkylenes (e.g, (CH₂)₁₀), C₁₁ alkylenes (e.g, (CH₂)₁₁), and C₁₂ alkylenes (e.g, (CH₂)₁₂).

Examples of a repeating unit of a polymer of Formula (II) include:

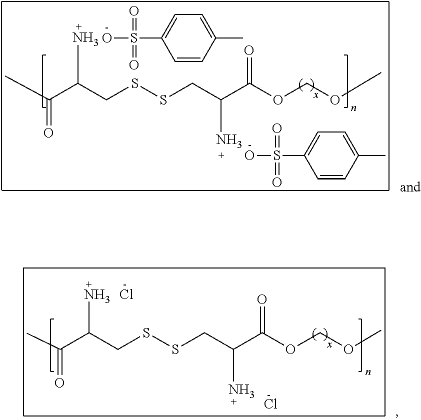

wherein x is an integer from 2 to 100.

In some embodiments, a polymer of Formula (I), Formula (Ia), and/or Formula (II) is a homopolymer comprising only the repeating unit according to the Formula. In some embodiments, a polymer of Formula (I), Formula (Ia), and/or Formula (II) is a copolymer comprising at least one repeating unit according to the Formula. For example, a polymer of Formula (I), Formula (Ia), and/or Formula (II) can be a copolymer comprising at least one repeating unit according to the Formula and PLGA (poly lactic (co-glycolic) acid).

In some embodiments, a polymer of Formula (I), Formula (Ia), and/or Formula (II) is a linear polymer. In some embodiments, a polymer of Formula (I), Formula (Ia), and/or Formula (II) is a branched polymer. In some embodiments, a polymer of Formula (I), Formula (Ia), and/or Formula (II) is a cross-linked polymer.

Terminal end groups for a polymer of Formula (I), Formula (Ia), and/or Formula (II) are known in the art, and can be any protecting groups, drugs, dyes, imaging reagents, targeting ligands, biological molecules which may terminate the polymerization process. For example, an N-terminal end group can be H, alkyl, alkenyl, alkynyl, cycloalkyl, aryl, heteroaryl, heterocyclyl, amide, sulfonamide, sulfamate, sulfinamide, or carbamate. A C-terminal end group can be carboxylic acid, ester, amide, or ketone of alkyl, alkenyl, alkynyl, cycloalkyl, aryl, heteroaryl, or heterocyclyl. For example, a drug molecule having an alcohol function, such as docetaxel, may be used as a C-terminal end group by attachment as an ester.

The molecular weight of a polymer of Formula (I), Formula (Ia), and/or Formula (II) can be determined by any means known in the art. In some embodiments, the number average molecular weight (Ma) of a polymer of Formula (I), Formula (Ia), and/or Formula (II) is determined by gel permeation chromatography (GPC). Typically, a polymer of Formula (I), Formula (Ia), and/or Formula (II) has from about 2 to about 100,000 repeating units. In some embodiments, the Mₙ of the polymer is in the range from about 600 to about 10,000,000 daltons, about 600 to about 150,000 daltons, about 600 to about 140,000 daltons, about 600 to about 130,000 daltons, about 600 to about 120,000 daltons, about 600 to about 110,000 daltons, about 600 to about 100,000 daltons, from about 600 to about 90,000 daltons, from about 600 to about 80,000 daltons, from about 600 to about 70,000 daltons, from about 600 to about 60,000 daltons, from about 600 to about 50,000 daltons, from about 600 to about 40,000 daltons, from about 600 to about 30,000 daltons, from about 600 to about 20,000 daltons, from about 600 to about 10,000 daltons, from about 600 to about 9,000 daltons, from about 600 to about 8,000 daltons, from about 600 to about 7,000 daltons, from about 600 to about 6,000 daltons, from about 600 to about 5,000 daltons, from about 600 to about 4,000 daltons, and/or from about 600 to about 3,000 daltons.

The polydispersity of a polymer of Formula (I), Formula (Ia), and/or Formula (II) can be determined by means known in the art. As used herein, the polydispersity or dispersity of a polymer measures the degree of uniformity in size of the polymer. In some embodiments, the polydispersity of a polymer of Formula (I), Formula (Ia), and/or Formula (II) is determined by gel permeation chromatography (GPC).

Without being limited to the following procedures, general schemes for the synthesis of a polymer of Formula (I), Formula (Ia), and/or Formula (II) include a polycondensation method that involves a cysteine monomer and a bis-activated ester or diacid chloride, as shown in the non-limiting example of Scheme 1, where x is the length of the methylene linker (e.g., x=1-100), and n is the number of repeating units (e.g., n=2-100,000).

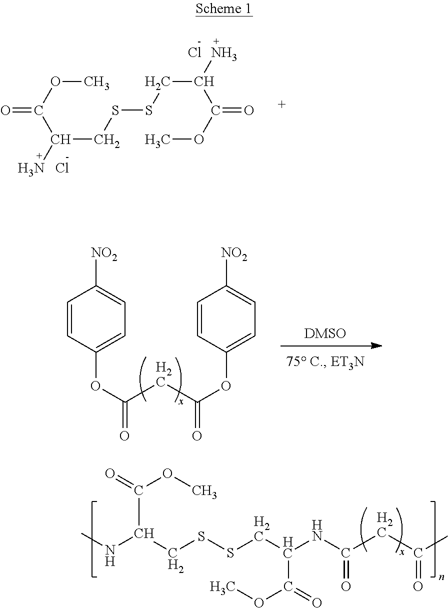

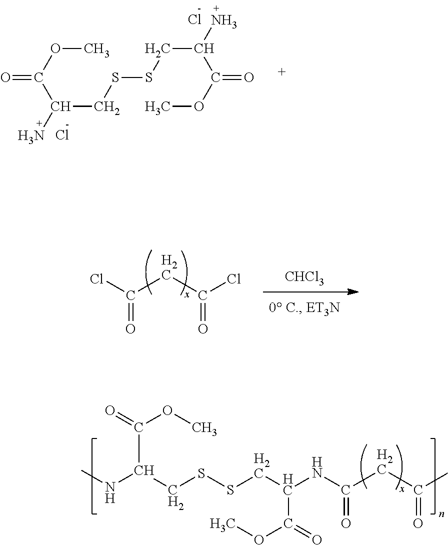

The polymers can also be synthesized by a polycondensation method that forms the cystine —S—S— bond simultaneous with polymerization, as illustrated in Scheme 2, where x is the length of the methylene linker (e.g., x=1-100), and n is the number of repeating units (e.g., n=2-100,000).

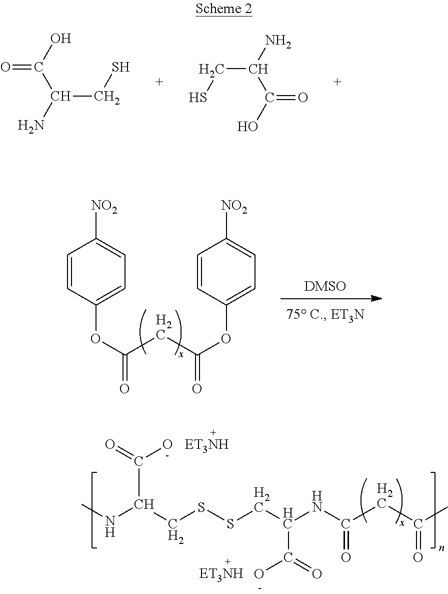

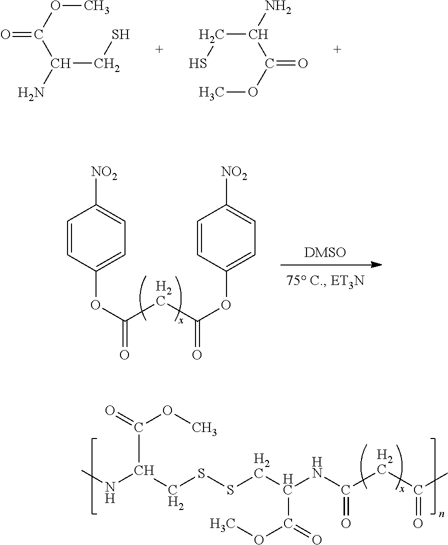

In some embodiments, the hydrophobic polymer is Cys-poly(disulfide amide) (Cys-PDSA) polymers were prepared by one-step polycondensation of (H-Cys-OMe)₂×2HCl and bis-fatty acid nitrophenol ester or dichloride of fatty acid in a variety of combinations. Prepared PDSAs are labeled as Cys-OMe-x or, equivalently Cys-xE, where x represents the number of methylene groups in the diacid repeating unit. Accordingly, the cysteine dimethyl ester copolymer with the respective blocks are coded as follows: succinyl chloride (Cys-OMe-2 or Cys-2E), adipoyl chloride (Cys-OMe-4 or Cys-4E), suberoyl chloride (Cys-OMe-6 or Cys-6E), sebacoyl chloride (Cys-OMe-8, or Cys-8E), and dodecanedioyl dichloride (Cys-OMe-10 or Cys-10E). The corresponding carboxylic acid polymers are coded with the cysteine carboxylic acid copolymer with the respective blocks as follows: succinyl chloride (Cys-OH-2), adipoyl chloride (Cys-OH-4), suberoyl chloride (Cys-OH-6), sebacoyl chloride (Cys-OH-8), and dodecanedioyl dichloride (Cys-OH-10).

In some embodiments, the core of the particle comprises a complexing agent. The complexing agent has a positive charge that is complementary to the overall negative charge of the p53 mRNA. The complexation allows the mRNA to self-assemble with the complexing agent, and that assembly is then successfully encapsulated in the hydrophobic polymeric core of the particle. In some embodiments, the complexing agent is amphiphilic (i.e., it contains both lipophilic and hydrophilic properties in the same molecule). The complexing agent can therefore comprise a segment that is hydrophobic and a segment that is hydrophilic.

A hydrophobic segment of an amphiphile can comprise, e.g., a hydrocarbon or a hydrocarbon that is substituted exclusively or predominantly with hydrophobic substituents such as halogen atoms. Typically, the hydrophobic segment can comprise a chain of 10, or more (e.g., 10, 11, 12, 13, 14, 15, 16, 17, 18, 19, 20 or more) carbon atoms. In some embodiments, the hydrophobic segment comprises an aliphatic chain, which in some embodiments can be branched and in some embodiments can be unbranched. In some embodiments, the hydrophobic segment comprises an aliphatic chain that is saturated. In some embodiments, the hydrophobic segment comprises an aliphatic chain that is unsaturated.

A hydrophilic segment of an amphiphile can comprise, e.g., one or more polar groups such as hydroxyl or ether groups. A hydrophilic segment of an amphiphile can comprise, e.g., one or more charged groups. A charged group can include a cation, e.g., ammonium or phosphonium groups. A charged group can include an anion, e.g., phosphate or sulfate groups.

A complexing agent within the core comprises a hydrophilic region and a hydrophobic region, and can comprise a variety of materials. In some embodiments, the complexing agent is negatively charged. In some embodiments, the complexing agent is positively charged. In some embodiments, the complexing agent comprises a phospholipid. In some embodiments, the complexing agent comprises a dendrimer. Dendrimers (also known as dendrons, arborols or cascade molecules) are repetitively branched molecules which can be classified by generation, which refers to the number of repeated branching cycles performed during synthesis. For example, poly(amidoamine) (PAMAM) is ethylenediamine reacted with methyl acrylate, and then another ethylenediamine to make a generation 0 (G0) PAMAM.

In some embodiments, the complexing agent is a cationic lipid or a cationic lipid-like material such as lipophilic moiety-modified amino dendrimer.

Suitable examples of lipophilic moieties with which an amino dendrimer may be modified include CₙH₂ₙ₋₁ alkyl chains where n is 8-22 (e.g., C₈, C₁₀, C₁₂, C₁₄, C₁₆, or C₁₈ groups), fatty acids and glycerides, and phospholipids. Examples of fatty acids include saturated and unsaturated fatty acids, such as linolenic acid, linoleic acid, myristic acid, stearic acid, palmitic acid, eicosanoic acid, and margaric acid. Examples of fatty glycerides and phospholipids include 1,2-dipalmitoyl-sn-glycero-3-phosphoethanolamine, 1,2-dioleoyl-sn-glycero-3-phosphoethanolamine, 1,2-dimyristoyl-sn-glycero-3-phosphoethanolamine, and 1,2-distearoyl-sn-glycero-3-phosphoethanolamine.

In some embodiments, the cationic lipid is selected from 1,2-dioleoyl-3-trimethylammonium-propane (DOTAP) and 1,2-di-O-octadecenyl-3-trimethylammonium propane (DOTMA); and the lipophilic moiety-modified amino dendrimer is selected from polypropylenimine tetramine dendrimer generation 1 modified with a lipophilic moiety, ethylenediamine core-poly (amidoamine) (PAMAM) generation 0 dendrimer (G0) modified with C14 (G0-C14 dendrimer); and ethylenediamine branched polyethyleneimine modified with lipophilic moiety.

In some embodiments, the weight ratio of the complexing agent to the p53-encoding mRNA in the core of the particle is from about 5 to about 20 (e.g., from 10 to 15).

In some embodiments, the complexing agent comprises one or more selected from the group consisting of: lecithin, an amino dendrimer (e.g., ethylenediamine core-poly (amidoamine) (PAMAM) generation 0 dendrimer (G0), ethylenediamine branched polyethylenimine (M_{w}˜ 800) (PEI), polypropylenimine tetramine dendrimer, generation 1 (DAB), and derivatives thereof, e.g., amino derivatives formed by reacting an amine group with an alkyl epoxide, e.g., G0-C14 dendrimer described in Xu, X. et al. Proc. Natl. Acad. Sci. U.S.A. 2013; 110:18638-43, which is hereby incorporated by reference in its entirety), a PEG-phospholipid (e.g., 14:0 PEG350 PE (1,2-dimyristoyl-sn-glycero-3-phosphoethanolamine-N-[methoxy(polyethylene glycol)-350]), 14:0 PEG350 PE (1,2-dipalmitoyl-sn-glycero-3-phosphoethanolamine-N-[methoxy(polyethylene glycol)-350]), 18:0 PEG350 PE (1,2-distearoyl-sn-glycero-3-phosphoethanolamine-N-[methoxy(polyethylene glycol)-350]), 18:1 PEG350 PE (1,2-dioleoyl-sn-glycero-3-phosphoethanolamine-N-[methoxy(polyethylene glycol)-350]), 14:0 PEG550 PE (1,2-dimyristoyl-sn-glycero-3-phosphoethanolamine-N-[methoxy(polyethylene glycol)-550]), 14:0 PEG550 PE (1,2-dipalmitoyl-sn-glycero-3-phosphoethanolamine-N-[methoxy(polyethylene glycol)-550]), 18:0 PEG550 PE (1,2-distearoyl-sn-glycero-3-phosphoethanolamine-N-[methoxy(polyethylene glycol)-550]), 18:1 PEG550 PE (1,2-dioleoyl-sn-glycero-3-phosphoethanolamine-N-[methoxy(polyethylene glycol)-550]), 14:0 PEG750 PE (1,2-dimyristoyl-sn-glycero-3-phosphoethanolamine-N-[methoxy(polyethylene glycol)-750]), 14:0 PEG750 PE (1,2-dipalmitoyl-sn-glycero-3-phosphoethanolamine-N-[methoxy(polyethylene glycol)-750]), 18:0 PEG750 PE (1,2-distearoyl-sn-glycero-3-phosphoethanolamine-N-[methoxy(polyethylene glycol)-750]), 18:1 PEG750 PE (1,2-dioleoyl-sn-glycero-3-phosphoethanolamine-N-[methoxy(polyethylene glycol)-750]), 14:0 PEG1000 PE (1,2-dimyristoyl-sn-glycero-3-phosphoethanolamine-N-[methoxy(polyethylene glycol)-1000]), 14:0 PEG1000 PE (1,2-dipalmitoyl-sn-glycero-3-phosphoethanolamine-N-[methoxy(polyethylene glycol)-1000]), 18:0 PEG1000 PE (1,2-distearoyl-sn-glycero-3-phosphoethanolamine-N-[methoxy(polyethylene glycol)-1000]) (DSPE-PEG1K), 18:1 PEG1000 PE (1,2-dioleoyl-sn-glycero-3-phosphoethanolamine-N-[methoxy(polyethylene glycol)-1000]), 14:0 PEG2000 PE (1,2-dimyristoyl-sn-glycero-3-phosphoethanolamine-N-[methoxy(polyethylene glycol)-2000]), 14:0 PEG2000 PE (1,2-dipalmitoyl-sn-glycero-3-phosphoethanolamine-N-[methoxy(polyethylene glycol)-2000]), 18:0 PEG2000 PE (1,2-distearoyl-sn-glycero-3-phosphoethanolamine-N-[methoxy(polyethylene glycol)-2000]) (DSPE-PEG2K), 18:1 PEG2000 PE (1,2-dioleoyl-sn-glycero-3-phosphoethanolamine-N-[methoxy(polyethylene glycol)-2000]), 14:0 PEG3000 PE (1,2-dimyristoyl-sn-glycero-3-phosphoethanolamine-N-[methoxy(polyethylene glycol)-3000]), 14:0 PEG3000 PE (1,2-dipalmitoyl-sn-glycero-3-phosphoethanolamine-N-[methoxy(polyethylene glycol)-3000]), 18:0 PEG3000 PE (1,2-distearoyl-sn-glycero-3-phosphoethanolamine-N-[methoxy(polyethylene glycol)-3000]) (DSPE-PEG3K), 18:1 PEG3000 PE (1,2-dioleoyl-sn-glycero-3-phosphoethanolamine-N-[methoxy(polyethylene glycol)-3000]), 14:0 PEG5000 PE (1,2-dimyristoyl-sn-glycero-3-phosphoethanolamine-N-[methoxy(polyethylene glycol)-5000]), 14:0 PEG5000 PE (1,2-dipalmitoyl-sn-glycero-3-phosphoethanolamine-N-[methoxy(polyethylene glycol)-5000]), 18:0 PEG5000 PE (1,2-distearoyl-sn-glycero-3-phosphoethanolamine-N-[methoxy(polyethylene glycol)-5000]) (DSPE-PEG5K), 18:1 PEG5000 PE (1,2-dioleoyl-sn-glycero-3-phosphoethanolamine-N-[methoxy(polyethylene glycol)-5000])), a PEG-ceramide (e.g., C8 PEG750 ceramide (N-octanoyl-sphingosine-1-{succinyl[methoxy(polyethylene glycol)750]}), C16 PEG750 ceramide (N-palmitoyl-sphingosine-1-{succinyl[methoxy(polyethylene glycol)750]}), C8 PEG2000 ceramide (N-octanoyl-sphingosine-1-{succinyl[methoxy(polyethylene glycol)2000]}), C16 PEG2000 ceramide (N-palmitoyl-sphingosine-1-{succinyl[methoxy(polyethylene glycol)2000]}), C8 PEG5000 ceramide (N-octanoyl-sphingosine-1-{succinyl[methoxy(polyethylene glycol)5000]}), C16 PEG5000 ceramide (N-palmitoyl-sphingosine-1-{succinyl[methoxy(polyethylene glycol)5000]}), an anionic lipid (e.g., 1,2-di-O-tetradecyl-sn-glycero-3-phospho-(1′-rac-glycerol), 1,2-dihexadecanoyl-sn-glycero-3-phospho-(1′-sn-glycerol)), and a cationic lipid (e.g., DC-cholesterol (38-[N-(N′,N′-dimethylaminoethane)-carbamoyl]cholesterol), 18:1 TAP (DOTAP) (1,2-dioleoyl-3-trimethylammonium-propane), 1-oleoyl-2-[6-[(7-nitro-2-1,3-benzoxadiazol-4-yl)amino]hexanoyl]-3-trimethylammonium propane, 14:0 TAP (1,2-dimyristoyl-3-trimethylammonium-propane), 16:0 TAP (1,2-dipalmitoyl-3-trimethylammonium-propane), 18:0 TAP (1,2-stearoyl-3-trimethylammonium-propane), DOTMA (1,2-di-O-octadecenyl-3-trimethylammonium propane), a phosphatidylcholine (e.g., 12:0 EPC (1,2-dilauroyl-sn-glycero-3-ethylphosphocholine), 14:0 EPC (1,2-dimyristoyl-sn-glycero-3-ethylphosphocholine), 14:1 EPC (1,2-dimyristoleoyl-sn-glycero-3-ethylphosphocholine), 16:0 EPC (1,2-dipalmitoyl-sn-glycero-3-ethylphosphocholine), 18:0 EPC (1,2-distearoyl-sn-glycero-3-ethylphosphocholine), 18:1 EPC (1,2-dioleoyl-sn-glycero-3-ethylphosphocholine), 16:0-18:1 EPC (1-palmitoyl-2-oleoyl-sn-glycero-3-ethylphosphocholine)). In some embodiments, the complexing agent consists essentially of, or consists of, one or more materials described herein.

The proportion of the complexing agent within the water-insoluble core in the particle depends on the characteristics of the complexing agent, the properties of the remainder of the core, and the application. In some embodiments, the complexing agent is in the core in an amount from about 1% by weight to about 50.0% by weight. The complexing agent is in the core in an amount from about 1% by weight to about 45% by weight, from about 1% by weight to about 40% by weight, from about 1% by weight to about 35% by weight, from about 1% by weight to about 30% by weight, from about 1% by weight to about 25% by weight, from about 1% by weight to about 20% by weight, from about 1% by weight to about 15% by weight, from about 10% by weight to about 45% by weight, from about 10% by weight to about 40% by weight, from about 10% by weight to about 35% by weight, from about 10% by weight to about 30% by weight, from about 10% by weight to about 25% by weight, from about 10% by weight to about 20% by weight, from about 10% by weight to about 15% by weight, from about 1% by weight to about 10% by weight, and/or from about 1% by weight to about 5% by weight. For example, the complexing agent can be present in about 2% by weight, about 5% by weight, about 10% by weight, about 15% by weight, about 20% by weight, about 25% by weight, about 30% by weight, about 35% by weight, about 40% by weight, about 45% by weight, or about 50% by weight.

In some embodiments, the particle comprises a shell attached to the core (e.g., covalently or non-covalently attached through electrostatic interactions, hydrophobic interactions, or Van der Waals forces). In some embodiments, the shell comprises an amphiphilic material. In some embodiments, the amphiphilic material can comprise a phospholipid and/or a poly(ethylene glycol). In some embodiments, the amphiphilic material comprises one or more selected from the group consisting of: lecithin, a neutral lipid (e.g., a diacyl glycerol (e.g., 8:0 DG (1,2-dioctanoyl-sn-glycerol), 10:0 DG (1,2-didecanoyl-sn-glycerol)), a sphingolipid (e.g., D-erythro-sphingosine and D-glucosyl-8-1,1′ N-octanoyl-D-erythro-sphingosine), a ceramide (e.g., N-butyroyl-D-erythro-sphingosine, N-octanoyl-D-erythro-sphingosine, N-stearoyl-D-erythro-sphingosine (C17 base))), a PEG-phospholipid (e.g., 14:0 PEG350 PE (1,2-dimyristoyl-sn-glycero-3-phosphoethanolamine-N-[methoxy(polyethylene glycol)-350]), 14:0 PEG350 PE (1,2-dipalmitoyl-sn-glycero-3-phosphoethanolamine-N-[methoxy(polyethylene glycol)-350]), 18:0 PEG350 PE (1,2-distearoyl-sn-glycero-3-phosphoethanolamine-N-[methoxy(polyethylene glycol)-350]), 18:1 PEG350 PE (1,2-dioleoyl-sn-glycero-3-phosphoethanolamine-N-[methoxy(polyethylene glycol)-350]), 14:0 PEG550 PE (1,2-dimyristoyl-sn-glycero-3-phosphoethanolamine-N-[methoxy(polyethylene glycol)-550]), 14:0 PEG550 PE (1,2-dipalmitoyl-sn-glycero-3-phosphoethanolamine-N-[methoxy(polyethylene glycol)-550]), 18:0 PEG550 PE (1,2-distearoyl-sn-glycero-3-phosphoethanolamine-N-[methoxy(polyethylene glycol)-550]), 18:1 PEG550 PE (1,2-dioleoyl-sn-glycero-3-phosphoethanolamine-N-[methoxy(polyethylene glycol)-550]), 14:0 PEG750 PE (1,2-dimyristoyl-sn-glycero-3-phosphoethanolamine-N-[methoxy(polyethylene glycol)-750]), 14:0 PEG750 PE (1,2-dipalmitoyl-sn-glycero-3-phosphoethanolamine-N-[methoxy(polyethylene glycol)-750]), 18:0 PEG750 PE (1,2-distearoyl-sn-glycero-3-phosphoethanolamine-N-[methoxy(polyethylene glycol)-750]), 18:1 PEG750 PE (1,2-dioleoyl-sn-glycero-3-phosphoethanolamine-N-[methoxy(polyethylene glycol)-750]), 14:0 PEG1000 PE (1,2-dimyristoyl-sn-glycero-3-phosphoethanolamine-N-[methoxy(polyethylene glycol)-1000]), 14:0 PEG1000 PE (1,2-dipalmitoyl-sn-glycero-3-phosphoethanolamine-N-[methoxy(polyethylene glycol)-1000]), 18:0 PEG1000 PE (1,2-distearoyl-sn-glycero-3-phosphoethanolamine-N-[methoxy(polyethylene glycol)-1000]) (DSPE-PEG1K), 18:1 PEG1000 PE (1,2-dioleoyl-sn-glycero-3-phosphoethanolamine-N-[methoxy(polyethylene glycol)-1000]), 14:0 PEG2000 PE (1,2-dimyristoyl-sn-glycero-3-phosphoethanolamine-N-[methoxy(polyethylene glycol)-2000]), 14:0 PEG2000 PE (1,2-dipalmitoyl-sn-glycero-3-phosphoethanolamine-N-[methoxy(polyethylene glycol)-2000]), 18:0 PEG2000 PE (1,2-distearoyl-sn-glycero-3-phosphoethanolamine-N-[methoxy(polyethylene glycol)-2000]) (DSPE-PEG2K), 18:1 PEG2000 PE (1,2-dioleoyl-sn-glycero-3-phosphoethanolamine-N-[methoxy(polyethylene glycol)-2000]), 14:0 PEG3000 PE (1,2-dimyristoyl-sn-glycero-3-phosphoethanolamine-N-[methoxy(polyethylene glycol)-3000]), 14:0 PEG3000 PE (1,2-dipalmitoyl-sn-glycero-3-phosphoethanolamine-N-[methoxy(polyethylene glycol)-3000]), 18:0 PEG3000 PE (1,2-distearoyl-sn-glycero-3-phosphoethanolamine-N-[methoxy(polyethylene glycol)-3000]) (DSPE-PEG3K), 18:1 PEG3000 PE (1,2-dioleoyl-sn-glycero-3-phosphoethanolamine-N-[methoxy(polyethylene glycol)-3000]), 14:0 PEG5000 PE (1,2-dimyristoyl-sn-glycero-3-phosphoethanolamine-N-[methoxy(polyethylene glycol)-5000]), 14:0 PEG5000 PE (1,2-dipalmitoyl-sn-glycero-3-phosphoethanolamine-N-[methoxy(polyethylene glycol)-5000]), 18:0 PEG5000 PE (1,2-distearoyl-sn-glycero-3-phosphoethanolamine-N-[methoxy(polyethylene glycol)-5000]) (DSPE-PEG5K), 18:1 PEG5000 PE (1,2-dioleoyl-sn-glycero-3-phosphoethanolamine-N-[methoxy(polyethylene glycol)-5000])), a PEG-ceramide (e.g., C8 PEG750 ceramide (N-octanoyl-sphingosine-1-{succinyl[methoxy(polyethylene glycol)750]}), C16 PEG750 ceramide (N-palmitoyl-sphingosine-1-{succinyl[methoxy(polyethylene glycol)750]}), C8 PEG2000 ceramide (N-octanoyl-sphingosine-1-{succinyl[methoxy(polyethylene glycol)2000]}), C16 PEG2000 ceramide (N-palmitoyl-sphingosine-1-{succinyl[methoxy(polyethylene glycol)2000]}), C8 PEG5000 ceramide (N-octanoyl-sphingosine-1-{succinyl[methoxy(polyethylene glycol)5000]}), C16 PEG5000 ceramide (N-palmitoyl-sphingosine-1-{succinyl[methoxy(polyethylene glycol)5000]}), an anionic lipid (e.g., 1,2-di-O-tetradecyl-sn-glycero-3-phospho-(1′-rac-glycerol), 1,2-dihexadecanoyl-sn-glycero-3-phospho-(1′-sn-glycerol)), and a cationic lipid (e.g., DC-cholesterol (38-[N-(N′,N′-dimethylaminoethane)-carbamoyl]cholesterol), 18:1 TAP (DOTAP) (1,2-dioleoyl-3-trimethylammonium-propane), 1-oleoyl-2-[6-[(7-nitro-2-1,3-benzoxadiazol-4-yl)amino]hexanoyl]-3-trimethylammonium propane, 14:0 TAP (1,2-dimyristoyl-3-trimethylammonium-propane), 16:0 TAP (1,2-dipalmitoyl-3-trimethylammonium-propane), 18:0 TAP (1,2-stearoyl-3-trimethylammonium-propane), DOTMA (1,2-di-O-octadecenyl-3-trimethylammonium propane), a phosphatidylcholine (e.g., 12:0 EPC (1,2-dilauroyl-sn-glycero-3-ethylphosphocholine), 14:0 EPC (1,2-dimyristoyl-sn-glycero-3-ethylphosphocholine), 14:1 EPC (1,2-dimyristoleoyl-sn-glycero-3-ethylphosphocholine), 16:0 EPC (1,2-dipalmitoyl-sn-glycero-3-ethylphosphocholine), 18:0 EPC (1,2-distearoyl-sn-glycero-3-ethylphosphocholine), 18:1 EPC (1,2-dioleoyl-sn-glycero-3-ethylphosphocholine), 16:0-18:1 EPC (1-palmitoyl-2-oleoyl-sn-glycero-3-ethylphosphocholine)). In some embodiments, the amphiphilic material comprises 1,2-distearoyl-sn-glycero-3-phosphoethanolamine-N-[methoxy(polyethylene glycol)-2000]. In some embodiments, the amphiphilic material comprises 1,2-distearoyl-sn-glycero-3-phosphoethanolamine-N-[methoxy(polyethylene glycol)-5000]. In some embodiments, the amphiphilic material comprises lecithin. In some embodiments, the amphiphilic material comprises 1,2-dimyristoyl-sn-glycero-3-phosphoethanolamine-N-[methoxy(polyethylene glycol)] (DMPE-PEG) or 1,2-distearoyl-sn-glycero-3-phosphoethanolamine-N-[methoxy(polyethylene glycol)] (DSPE-PEG), or any combination thereof. In some embodiments, the amphiphilic material consists essentially of, or consists of, one or more materials described herein.

The proportion of the amphiphilic material relative to the core in the particle depends on the characteristics of the amphiphilic material, the properties of the core, and the application. In some embodiments, the amphiphilic material is in the range from about 1% by weight to about 50.0% by weight compared with the weight of the core. The amphiphilic material can be in the range from about 1% by weight to about 45% by weight, from about 1% by weight to about 40% by weight, from about 1% by weight to about 35% by weight, from about 1% by weight to about 30% by weight, from about 1% by weight to about 25% by weight, from about 1% by weight to about 20% by weight, from about 1% by weight to about 15% by weight, from about 1% by weight to about 10% by weight, and/or from about 1% by weight to about 5% by weight compared with the weight of the core. For example, the amphiphilic material can be about 2% by weight, about 5% by weight, about 10% by weight, about 15% by weight, about 20% by weight, about 25% by weight, about 30% by weight, about 35% by weight, about 40% by weight, about 45% by weight, or about 50% by weight compared with the weight of the core.

In some embodiments, the particles of the present disclosure can be prepared according to the methods similar to those described in WO 2018/089688, US20170362388, and US20170304213, which are incorporated herein by reference in their entirety.

Pharmaceutical Compositions and Formulations

The present application also provides pharmaceutical compositions comprising an effective amount of an active ingredient as disclosed herein, or a pharmaceutically acceptable salt thereof, and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

Pharmaceutically Acceptable Salts

In some embodiments, a salt of any one of the compounds described herein (e.g., a small-molecule anticancer agent) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

Dosages and Regimens

Any of the compositions of the present disclosure contain the active ingredient (e.g., p53 mRNA, small-molecule therapeutic agent) in an effective amount (e.g., a therapeutically effective amount).

Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician (e.g., oncologist).

In some embodiments, an effective amount (e.g., therapeutically effective amount) of any one of the active ingredients of the present application (e.g., p53 mRNA, small-molecule therapeutic agent), or a pharmaceutically acceptable salt thereof, can range, for example, from about from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of mTOR inhibitor (e.g., everolimus), or a pharmaceutically acceptable salt thereof, is from about 0, 25 mg to about 10 mg, e.g., about 0.25 mg, about 0.5 mg, about 0.75 mg, about 2 mg, about 2.5 mg, about 3 mg, about 5 mg, about 7.5 mg, or about 10 mg.

In some embodiments, an effective amount of a DMA alkylating agent (e.g., cisplatin), or a pharmaceutically acceptable salt thereof, is about 1 mg/kg to about 10 mg/kg (e.g., 1 mg/kg, 3 mg/kg, or 8 mg/kg).

In some embodiments, an effective amount of AMPK activator (e.g., metformin), or a pharmaceutically acceptable salt thereof, is from about 250 mg to about 1,000 mg, e.g., about 500 mg, about 750 mg, about 850 mg, or about 1,000 mg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

In the method of treating cancer, the p53 mRNA-containing vehicle (e.g., nanoparticle composition) and the small-molecule anticancer agent (e.g., mTOR inhibitor, DNA alkylating agent, or AMPK activator) may be administered to the subject simultaneously (e.g., in the same dosage form or in separate dosage forms), or consecutively (e.g., before or after one another, in separate dosage forms).

Additional Therapeutic Agents

In some embodiments, at least one additional therapeutic agent can be administered to the patient. In some embodiments, the therapeutic agent is an anticancer agent. Suitable examples of the anticancer agents include abarelix, ado-trastuzumab emtansine, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan, calusterone, capecitabine, carboplatin, carmustine, cetuximab, chlorambucil, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, emtansine, epirubicin, eribulin, erlotinib, estramustine, etoposide phosphate, etoposide, everolimus, exemestane, fentanyl citrate, filgrastim, floxuridine, fludarabine, fluorouracil, fruquintinib, fulvestrant, gefitinib, gemcitabine, gemtuzumab ozogamicin, goserelin acetate, histrelin acetate, ibritumomab tiuxetan, idarubicin, ifosfamide, imatinib mesylate, interferon α2a, irinotecan, ixabepilone, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, paclitaxel, paclitaxel albumin-stabilized nanoparticle formulation, pamidronate, panitumumab, pegaspargase, pegfilgrastim, pemetrexed disodium, pentostatin, pertuzuma, pipobroman, plicamycin, procarbazine, quinacrine, rasburicase, rituximab, sorafenib, streptozocin, sulfatinib, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, volitinib, vorinostat, and zoledronate, or a pharmaceutically acceptable salt thereof. In some embodiments, the anticancer agent is a proteasome inhibitor (e.g., bortezomib, carfilzomib, or ixazomib).

In some embodiments, the additional therapeutic agent includes a pain relief agent (e.g., a nonsteroidal anti-inflammatory drug such as celecoxib or rofecoxib), an antinausea agent, a cardioprotective drug (e.g., dexrazoxane, ACE-inhibitors, diuretics, cardiac glycosides), a cholesterol lowering drug, a revascularization drug, a beta-blocker (e.g., acebutolol, atenolol, bisoprolol, metoprolol, nadolol, nebivolol, or propranolol), or an angiotensin receptor blocker (also called ARBs or angiotensin II inhibitors) (e.g., azilsartan, candesartan, eprosartan, irbesartan, losartan, olmesartan, telmisartan, or valsartan), or a pharmaceutically acceptable salt thereof.

In the method of treating cancer, the combination within the present claims and the additional therapeutic agent may be administered to the subject simultaneously (e.g., in the same dosage form or in separate dosage forms), or consecutively (e.g., before or after one another).

In some embodiments, the combination within the present claims may be administered to the subject in combination with one or more additional anti-cancer therapies selected from: surgery, biological therapy, radiation therapy, anti-angiogenesis therapy, immunotherapy, adoptive transfer of effector cells, gene therapy, and hormonal therapy.

### Definitions

For the terms “e.g.” and “such as,” and grammatical equivalents thereof, the phrase “and without limitation” is understood to follow unless explicitly stated otherwise.

As used herein, the singular forms “a,” “an,” and “the” include plural referents unless the context clearly dictates otherwise.

As used herein, the term “about” means “approximately” (e.g., plus or minus approximately 10% of the indicated value).

As used herein, “alkyl” refers to a saturated hydrocarbon chain that may be a straight chain or a branched chain. An alkyl group formally corresponds to an alkane with one C—H bond replaced by the point of attachment of the alkyl group to the remainder of the polymer. The term “(C_{x-y})alkyl” (wherein x and y are integers) by itself or as part of another substituent means, unless otherwise stated, an alkyl group containing from x to y carbon atoms. For example, a (C₁₋₆)alkyl group may have from one to six (inclusive) carbon atoms in it. Examples of (C₁₋₆)alkyl groups include, but are not limited to, methyl, ethyl, n-propyl, n-butyl, n-pentyl, n-hexyl, isopropyl, isobutyl, sec-butyl, tert-butyl, isopentyl, neopentyl and isohexyl. The (C_{x-y})alkyl groups include (C₁₋₆)alkyl, (C₁₋₄)alkyl and (C₁₋₃)alkyl. The term “(C_{x-y})alkylene” (wherein x and y are integers) refers to an alkylene group containing from x to y carbon atoms. An alkylene group formally corresponds to an alkane with two C—H bonds replaced by points of attachment of the alkylene group to the remainder of the polymer. Examples are divalent straight hydrocarbon groups consisting of methylene groups, such as, —CH₂—, —CH₂CH₂—, —CH₂CH₂CH₂—. The (C_{x-y})alkylene groups include (C₁₋₆)alkylene and (C₁₋₃)alkylene.

As used herein, “alkenyl” refers to an unsaturated hydrocarbon chain that includes a C═C double bond. An alkenyl group formally corresponds to an alkene with one C—H bond replaced by the point of attachment of the alkenyl group to the remainder of the polymer. The term “(C_{x-y})alkenyl” (wherein x and y are integers) denotes a radical containing x to y carbons, wherein at least one carbon-carbon double bond is present (therefore x must be at least 2). Some embodiments are 2 to 4 carbons, some embodiments are 2 to 3 carbons and some embodiments have 2 carbons. Alkenyl groups may include both E and Z stereoisomers. An alkenyl group can include more than one double bond. Examples of alkenyl groups include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl, and the like.

The term “(C_{x-y})alkenylene” (wherein x and y are integers) refers to an alkenylene group containing from x to y carbon atoms. An alkenylene group formally corresponds to an alkene with two C—H bonds replaced by points of attachment of the alkenylene group to the remainder of the polymer. Examples are divalent straight hydrocarbon groups consisting of alkenyl groups, such as —HC═CH— and —HC═CH—CH₂—. The (C_{x-y})alkenylene groups include (C₂₋₆)alkenylene and (C₂₋₄)alkenylene.

The term “(C_{x-y})heteroalkylene” (wherein x and y are integers) refers to a heteroalkylene group containing from x to y carbon atoms. A heteroalkylene group corresponds to an alkylene group wherein one or more of the carbon atoms have been replaced by a heteroatom. The heteroatoms may be independently selected from the group consisting of O, N and S. A divalent heteroatom (e.g., O or S) replaces a methylene group of the alkylene —CH₂—, and a trivalent heteroatom (e.g., N) replaces a methine group. Examples are divalent straight hydrocarbon groups consisting of methylene groups, such as, —CH₂—, —CH₂CH₂—, —CH₂CH₂CH₂—. The (C_{x-y})alkylene groups include (C₁₋₆)heteroalkylene and (C₁₋₃)heteroalkylene.

As used herein, “alkynyl” refers to an unsaturated hydrocarbon chain that includes a C≡C triple bond. An alkynyl group formally corresponds to an alkyne with one C—H bond replaced by the point of attachment of the alkyl group to the remainder of the polymer. The term “(C_{x-y})alkynyl” (wherein x and y are integers) denotes a radical containing x to y carbons, wherein at least one carbon-carbon triple bond is present (therefore x must be at least 2). Some embodiments are 2 to 4 carbons, some embodiments are 2 to 3 carbons and some embodiments have 2 carbons. Examples of an alkynyl include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “(C_{x-y})alkynylene” (wherein x and y are integers) refers to an alkynylene group containing from x to y carbon atoms. An alkynylene group formally corresponds to an alkyne with two C—H bonds replaced by points of attachment of the alkynylene group to the remainder of the polymer. Examples are divalent straight hydrocarbon groups consisting of alkynyl groups, such as —C≡C— and —C≡C—CH₂—. The (C_{x-y})alkylene groups include (C₂₋₆)alkynylene and (C₂₋₃)alkynylene.

The term “alkoxy” refers to an alkyl group having an oxygen attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “cycloalkyl”, employed alone or in combination with other terms, refers to a non-aromatic, saturated, monocyclic, bicyclic or polycyclic hydrocarbon ring system, including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃0.7). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally substituted by oxo or sulfido. Cycloalkyl groups also include cycloalkylidenes. Example cycloalkyl groups include cyclopropyl, cyclobutyl, cyclopentyl, cyclohexyl, cyclopentenyl, cyclohexenyl, cyclohexadienyl, norbornyl, norpinyl, bicyclo[2.1.1]hexanyl, bicyclo[1.1.1]pentanyl and the like. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like, e.g., indanyl or tetrahydronaphthyl. A cycloalkyl group containing a fused aromatic ring can be attached through any ring-forming atom including a ring-forming atom of the fused aromatic ring.

The term “heterocycloalkyl”, employed alone or in combination with other terms, refers to non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members or 4-6 ring members. Included in heterocycloalkyl are monocyclic 4-, 5-, 6- and 7-membered heterocycloalkyl groups. Heterocycloalkyl groups can include mono- or bicyclic (e.g., having two fused or bridged rings) ring systems. In some embodiments, the heterocycloalkyl group is a monocyclic group having 1, 2 or 3 heteroatoms independently selected from nitrogen, sulfur and oxygen. Examples of heterocycloalkyl groups include azetidine, pyrrolidine, piperidine, piperazine, morpholine, thiomorpholine, pyran, azepane, tetrahydropyran, tetrahydrofuran, dihydropyran, dihydrofuran and the like. Ring-forming carbon atoms and heteroatoms of a heterocycloalkyl group can be optionally substituted by oxo or sulfido (e.g., C(═O), S(═O), C(S) or S(═O)₂, etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 1, 2, 3, 4-tetrahydroquinoline, dihydrobenzofuran, azetidine, azepane, diazepan (e.g., 1,4-diazepan), pyrrolidine, piperidine, piperazine, morpholine, thiomorpholine, pyran, tetrahydrofuran and di- and tetra-hydropyran.

As used herein, “halo” or “halogen” refers to —F, —Cl, —Br and —I.

As used herein, “aryl,” employed alone or in combination with other terms, refers to an aromatic hydrocarbon group. The aryl group may be composed of, e.g., monocyclic or bicyclic rings and may contain, e.g., from 6 to 12 carbons in the ring, such as phenyl, biphenyl and naphthyl. The term “(C_{x-y})aryl” (wherein x and y are integers) denotes an aryl group containing from x to y ring carbon atoms. Examples of a (C₆₋₁₄)aryl group include, but are not limited to, phenyl, α-naphthyl, β-naphthyl, biphenyl, anthryl, tetrahydronaphthyl, fluorenyl, indanyl, biphenylenyl and acenanaphthyl. Examples of a C₆₋₁₀ aryl group include, but are not limited to, phenyl, α-naphthyl, β-naphthyl, biphenyl and tetrahydronaphthyl.

An aryl group can be unsubstituted or substituted. A substituted aryl group can be substituted with one or more groups, e.g., 1, 2 or 3 groups, including: (C₁₋₆)alkyl, (C₂₋₆)alkenyl, (C₂₋₆)alkynyl, halogen, (C₁₋₆)haloalkyl, —CN, —NO₂, —C(═O)R, —C(═O)OR, —C(═O)NR₂, —C(═NR)NR₂, —NR₂, —NRC(═O)R, —NRC(═O)O(C₁₋₆)alkyl, —NRC(═O)NR₂, —NRC(═NR)NR₂, —NRSO₂R, —OR, —O(C₁₋₆)haloalkyl, —OC(═O)R, —OC(═O)O(C₁₋₆)alkyl, —OC(═O)NR₂, —SR, —S(O)R, —SO₂R, —OSO₂(C₁₋₆)alkyl, —SO₂NR₂, —(C₁₋₆)alkylene-CN, —(C₁₋₆)alkylene-C(═O)OR, —(C₁₋₆)alkylene-C(═O)NR₂, —(C₁₋₆)alkylene-OR, —(C₁₋₆)alkylene-OC(═O)R, —(C₁₋₆)alkylene-NR₂, —(C₁₋₆)alkylene-NRC(═O)R, —NR(C₁₋₆)alkylene-C(═O)OR, —NR(C₁₋₆)alkylene-C(═O)NR₂, —NR(C₂₋₆)alkylene-OR, —NR(C₂₋₆)alkylene-OC(═O)R, —NR(C₂₋₆)alkylene-NR₂, —NR(C₂₋₆)alkylene-NRC(═O)R, —O(C₁₋₆)alkylene-C(═O)OR, —O(C₁₋₆)alkylene-C(═O)NR₂, —O(C₂₋₆)alkylene-OR, —O(C₂₋₆)alkylene-OC(═O)R, —O(C₂₋₆)alkylene-NR₂ and —O(C₂₋₆)alkylene-NRC(═O)R, wherein each R group is hydrogen or (C₁₋₆ alkyl).

The terms “heteroaryl” or “heteroaromatic” as used herein refer to an aromatic ring system having at least one heteroatom in at least one ring, and from 2 to 9 carbon atoms in the ring system. The heteroaryl group has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaryls include furyl, thienyl, pyridyl, oxazolyl, pyrrolyl, indolyl, quinolinyl or isoquinolinyl, and the like. The heteroatoms of the heteroaryl ring system can include heteroatoms selected from one or more of nitrogen, oxygen and sulfur.

Examples of heteroaryl groups include: pyridyl, pyrazinyl, pyrimidinyl, particularly 2- and 4-pyrimidinyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heteroaryls include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1, 5-naphthyridinyl, 1, 8-naphthyridinyl, 1,4-benzodioxanyl, coumarin, dihydrocoumarin, benzofuryl, particularly 3-, 4-, 5-, 6- and 7-benzofuryl, 2, 3-dihydrobenzofuryl, 1,2-benzisoxazolyl, benzothienyl, particularly 3-, 4-, 5-, 6- and 7-benzothienyl, benzoxazolyl, benzthiazolyl, purinyl, benzimidazolyl, and benztriazolyl.

A heteroaryl group can be unsubstituted or substituted. A substituted heteroaryl group can be substituted with one or more groups, e.g., 1, 2 or 3 groups, including: (C₁₋₆)alkyl, (C₂₋₆)alkenyl, (C₂₋₆)alkynyl, halogen, (C₁₋₆)haloalkyl, —CN, —NO₂, —C(═O)R, —C(═O)OR, —C(═O)NR₂, —C(═NR)NR₂, —NR₂, —NRC(═O)R, —NRC(═O)O(C₁₋₆)alkyl, —NRC(═O)NR₂, —NRC(═NR)NR₂, —NRSO₂R, —OR, —O(C₁₋₆)haloalkyl, —OC(═O)R, —OC(═O)O(C₁₋₆)alkyl, —OC(═O)NR₂, —SR, —S(O)R, —SO₂R, —OSO₂(C₁₋₆)alkyl, —SO₂NR₂, —(C₁₋₆)alkylene-CN, —(C₁₋₆)alkylene-C(═O)OR, —(C₁₋₆)alkylene-C(═O)NR₂, —(C₁₋₆)alkylene-OR, —(C₁₋₆)alkylene-OC(═O)R, —(C₁₋₆)alkylene-NR₂, —(C₁₋₆)alkylene-NRC(═O)R, —NR(C₁₋₆)alkylene-C(═O)OR, —NR(C₁₋₆)alkylene-C(═O)NR₂, —NR(C₂₋₆)alkylene-OR, —NR(C₂₋₆)alkylene-OC(═O)R, —NR(C₂₋₆)alkylene-NR₂, —NR(C₂₋₆)alkylene-NRC(═O)R, —O(C₁₋₆)alkylene-C(═O)OR, —O(C₁₋₆)alkylene-C(═O)NR₂, —O(C₂₋₆)alkylene-OR, —O(C₂₋₆)alkylene-OC(═O)R, —O(C₂₋₆)alkylene-NR₂ and —O(C₂₋₆)alkylene-NRC(═O)R, wherein each R group is hydrogen or (C₁₋₆ alkyl).

The term “Encapsulation efficiency” (EE) as used herein is the ratio of the amount of drug that is encapsulated by the particles (e.g., nanoparticles) to the initial amount of drug used in preparation of the particle.

The term “Loading capacity” (LC) or “loading efficiency” (LE) as used herein is the mass fraction of drug that is encapsulated to the total mass of the particles (e.g., nanoparticles).

A “polymer,” as used herein, is given its ordinary meaning as used in the art, i.e., a molecular structure including one or more repeat units (monomers), connected by covalent bonds. The polymer may be a copolymer. The repeat units forming the copolymer may be arranged in any fashion. For example, the repeat units may be arranged in a random order, in an alternating order, or as a “block” copolymer, i.e., including one or more regions each including a first repeat unit (e.g., a first block), and one or more regions each including a second repeat unit (e.g., a second block), etc. Block copolymers may have two (a diblock copolymer), three (a triblock copolymer), or more numbers of distinct blocks.

A “copolymer” herein refers to more than one type of repeat unit present within the polymer defined below.

A “particle” refers to any entity having a diameter of less than 10 microns (m). Typically, particles have a longest dimension (e.g., diameter) of 1000 nm or less. In some embodiments, particles have a diameter of 300 nm or less. Particles include microparticles, nanoparticles, and picoparticles. In some embodiments, particles can be a polymeric particle, non-polymeric particle (e.g., a metal particle, quantum dot, ceramic, inorganic material, bone, etc.), liposomes, micelles, hybrids thereof, and/or combinations thereof. As used herein, the term “nanoparticle” refers to any particle having a diameter of less than 1000 nm. In preferred embodiments, a nanoparticle is a polymeric particle that can be formed using a solvent emulsion, spray drying, or precipitation in bulk or microfluids, wherein the solvent is removed to no more than an insignificant residue, leaving a solid (which may, or may not, be hollow or have a liquid filled interior) polymeric particle, unlike a micelle whose form is dependent upon being present in an aqueous solution.

The term “particle size” (or “nanoparticle size” or “microparticle size”) as used herein refers to the median size in a distribution of nanoparticles or microparticles. The median size is determined from the average linear dimension of individual nanoparticles, for example, the diameter of a spherical nanoparticle. Size may be determined by any number of methods in the art, including dynamic light scattering (DLS) and transmission electron microscopy (TEM) techniques.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound.

The terms “inhibit” and “reduce” means to reduce or decrease in activity or expression. This can be a complete inhibition or reduction of activity or expression, or a partial inhibition or reduction. Inhibition or reduction can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

Materials and Methods

Experimental design. This experiment aimed to explore a mRNA-based strategy for restoring tumor suppressor p53 in p53-null HCC and NSCLC cells, and to evaluate whether p53 reactivation would sensitize these tumor cells to mTOR inhibition for more effective combination treatment. We addressed this objective by i) developing a redox-responsive p53-mRNANP platform that showed the feasibility of p53 restoration in p53-deficient Hep3B and H1299 cells; ii) demonstrating anti-tumor effects of the p53-mRNANPs that can induce cell apoptosis and G1-phase cell cycle arrest; and iii) revealing that p53 reactivation can sensitize tumor cells to mTOR inhibitor everolimus. The therapeutic efficacy and safety of the combination of p53-mRNA NPs with everolimus were thoroughly evaluated in vivo. Four animal models, including xenograft models of p53-null HCC and NSCLC, orthotopic model of p53-null HCC, and disseminated model of p53-null NSCLC, were used to evaluate anti-tumor effects of this combinatorial strategy. The animals were randomly assigned to the study groups. The experimentalists were not blinded during the study.

Animals. All the in vivo studies were conducted following the animal protocols approved by the Institutional Animal Care and Use Committees on animal care (Brigham and Women's Hospital and Hangzhou Normal University). The animal studies were performed under strict regulations and pathogen-free conditions in the animal facilities of Brigham and Women's Hospital or Hangzhou Normal University. Female athymic nude mice (4-6 weeks old), wild-type BALB/c mice (6 weeks old), and female C57BL/6 mice (4 weeks old) were purchased from Charles River Laboratories or Zhejiang Medical Academy Animal Center. Mice were raised for at least one week before the start of the experiments to acclimatize them to the environment and food of the animal facilities.

Pharmacokinetic (PK) and biodistribution (BioD) studies. For the in vivo PK study, healthy BALB/c mice (6 weeks old, n=3 per group) were injected intravenously with naked Cy5-mRNA, Cy5-mRNANP₂₅, Cy5-mRNANP₅₀, or Cy5-mRNANP₇₅ via tail vein. At predetermined time intervals (0, 0.5, 1, 2, 4, 8, 12, and 24 hours), retro-orbital vein blood was obtained in a heparin-coated capillary tube. The wound was gently pressed for one minute to stop the bleeding. Fluorescence intensity of Cy5-mRNA was measured by a microplate reader. PK was assessed by measuring the percentage of Cy5-mRNA in blood at these time points after getting rid of the background and normalization to the initial time point (0 h). For the BioD study, p53-null Hep3B xenograft-bearing athymic nude mice were injected intravenously with naked Cy5-mRNA, Cy5-mRNANP₂₅, Cy5-mRNANP₅₀, or Cy5-mRNA NP₇s (at an mRNA dose of 750 μg per kg of animal weight) via tail vein (n=3 per group). After 24 hours, all the mice were sacrificed, and the dissected organs and tumors were visualized using a Syngene PXi imaging system (Synoptics Ltd).

In vivo therapeutic efficacy in p53-null HCC xenograft tumor model. To establish the HCC xenograft tumor model, ˜1×10⁷ p53-null Hep3B liver cancer cells in 100 μl of PBS mixed with 100 μl of Matrigel (BD Biosciences) were implanted subcutaneously (s.c.) on the right flank (near the liver) of female athymic nude mice. Mice were monitored for tumor growth every other day according to the animal protocol. When the tumor volume reached about ˜100 mm³, the mice were randomly divided into five groups (n=5), which received treatment with PBS, EGFP-mRNA NPs, everolimus, p53-mRNA NPs, or p53-mRNA NPs+everolimus. The mRNANPs used for the in vivo therapeutic studies had 75% (w/w %) of DSPE-PEG in lipid-PEG layer. The human p53-mRNA sequence is shown in FIG. 57. The EGFP-mRNANPs or p53-mRNANPs were injected via tail vein at an mRNA dose of 750 μg/kg, whereas the everolimus was orally administered at 5 mg/kg every three days over six rounds of treatment. The day that first treatment was performed was designated as Day 0. Tumor size was measured using a caliper every three days from Day 0 to Day 33, and the average tumor volume (mm³) was calculated as: 4π/3×(tumor length/2)×(tumor width/2)². Relative tumor volume (%) was calculated and presented according to a reported method (96). The largest tumor volume from the mouse at the end of this study was defined as 100%. The body weights of all the mice were also recorded over this period.

In vivo therapeutic efficacy in p53-null NSCLC xenograft tumor model. To establish the xenograft tumor mouse model, ˜5×10⁶ H1299 lung cancer cells in 100 μl of PBS mixed with 100 μl of Matrigel (BD Biosciences) were implanted s.c. on the left fore (near lung) of female athymic nude mice. Mice were monitored for tumor growth every other day according to the animal protocol. When the tumor volume reached about −100 mm³, the mice were randomly divided into five groups (n=5), which received treatment with PBS, EGFP-mRNANPs, everolimus, p53-mRNANPs, or p53-mRNANPs together with everolimus. The engineered mRNA NPs used for the in vivo therapeutic studies have 75% (w/w %) of DSPE-PEG in lipid-PEG layer. The EGFP-mRNA NPs or p53-mRNA NPs were injected via tail vein at an mRNA dose of 750 μg/kg, whereas the everolimus was orally administered at 5 mg/kg every three days for six treatments. The day that first treatment performed was designated as Day 0. Tumor size was measured using a caliper every three days from Day 0 to Day 18, and the average tumor volume (mm³) was calculated as: 4π/3×(tumor length/2)×(tumor width/2)². Relative tumor volume (%) was calculated and presented according to a reported method (96). The largest tumor volume from the mouse at the end of this study was defined as 100%.

In vivo therapeutic efficacy of murine p53-mRNA NPs in immunocompetent mice. To establish the immunocompetent mouse tumor model, ˜1×10⁶ of p53-null RIL-175 mouse HCC cells in 100 μl of PBS mixed with 100 μl of Matrigel (BD Biosciences) were implanted s.c. on the right flank (near the liver) of female C57BL/6 mice. Mice were monitored for tumor growth every other day according to the animal protocol. When the tumor volume reached about −100 mm³, the mice were randomly divided into three groups (n=5), which received treatment with PBS, EGFP-mRNANPs, or murine p53-mRNANPs. The mRNA NPs used for the in vivo therapeutic studies had 75% (w/w %) of DSPE-PEG in lipid-PEG layer. The mouse p53-mRNA sequence is shown in FIG. 57. The EGFP-mRNA NPs or murine p53-mRNANPs were intravenously injected via tail vein at an mRNA dose of 750 μg/kg, every three days over six rounds of treatment. The day that first treatment was performed was designated as Day 0. Tumor size was measured using a caliper every three days from Day 0 to Day 18, and the average tumor volume (mm³) was calculated as: 4π/3×(tumor length/2)×(tumor width/2)². Relative tumor volume (%) was calculated and presented according to a reported method (96).

In vivo mechanisms underlying the p53-mRNA NP-mediated sensitization to everolimus. To verify the in vivo mechanisms underlying this p53-mRNA NP-mediated strategy, mice bearing p53-null Hep3B liver xenografts were treated with p53-mRNA NPs via tail vein injection at an mRNA dose of 750 μg/kg every three days for three rounds of treatment. The mice were sacrificed at 12, 24, 48, or 60 hours after the last injection of p53-mRNANPs, and the tumors were harvested for sections. Mice bearing p53-null Hep3B liver xenografts and intravenously injected with PBS were used as controls and sacrificed at 60 hours after the last injection. The expression of p53 and C-CAS3 was monitored via IF detection. Moreover, tumor sections from both the PBS group and p53-mRNANP group (60 hours after the last injection) were analyzed by IHC. The expression of p53, tumor cell apoptosis markers (BAX, C-CAS3), and proliferation markers (Ki67 and PCNA) was further assessed. In addition, tumors obtained from all the groups (control, EGFP-mRNANPs, everolimus, p53-mRNA NPs, or p53-mRNA NPs+everolimus) in the above-mentioned therapeutic study using p53-null Hep3B liver xenograft model were further sectioned for a TUNEL apoptosis assay and lysed for WB studies to detect the expression of p53, LC3B-2, BECN1, p62, p-4EBP1, C-CAS9, and C-CAS3.

In vivo therapeutic efficacy in p53-null orthotopic HCC model. To establish the orthotopic HCC model, luciferase-expressing Hep3B (Hep3B-Luc) cells were used. Six-week-old female athymic nude mice were obtained from Zhejiang Medical Academy Animal Center. Animal studies were conducted following the protocol approved by the Institutional Animal Ethics Committee of Hangzhou Normal University. First, anterior abdominal exposure was made and a cotton swab with iodine volts was used to sterilize this area. A one-centimeter-long midline incision was made along the anterior abdominal wall below the xiphoid after anesthesia by isoflurane, and ˜5×10⁶ p53-null Hep3B-Luc cells in 50 μl of PBS were injected into the left lobe of the livers of the athymic nude mice (30 in total). The injection depth was not deeper than 2 mm. The inner and outer layers of the abdominal cavity were sutured one by one after tumor cell inoculation. Three weeks later, 15 mice (incidence rate of orthotopic HCC model: 50%) were randomly assigned to five groups (n=3 per group), which received treatment with PBS, EGFP-mRNANPs, everolimus, p53-mRNANPs, or p53-mRNA NPs together with everolimus. The EGFP-mRNA NPs or p53-mRNA NPs were injected via tail vein at an mRNA dose of 750 μg/kg, whereas everolimus was orally administered at 5 mg/kg every three days for four rounds of treatment. The first treatment was performed at Day 0. On Day 12, all the mice were sacrificed. Mice were monitored for tumor growth by bioluminescent in vivo imaging every 6 days (Day 0, 6, and 12). To do this, these mice were injected intraperitoneally with 150 mg/kg D-luciferin substrate (PerkinElmer, Catalog #122799) and imaged by an IVIS Lumina S5 (PerkinElmer) imaging system.

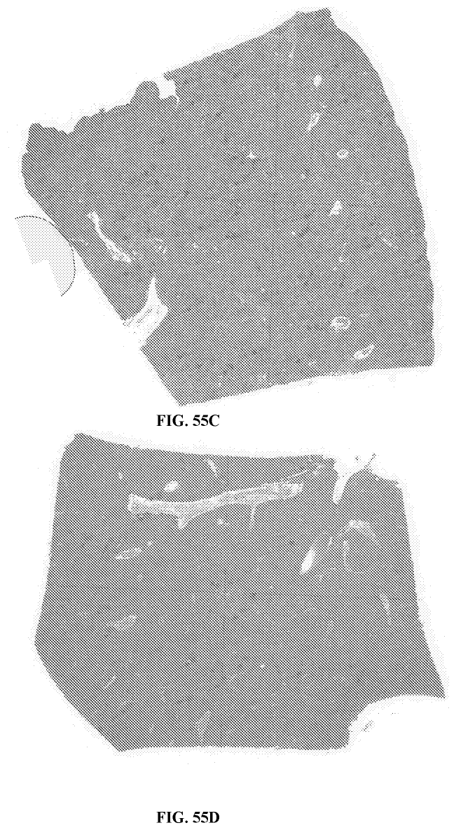

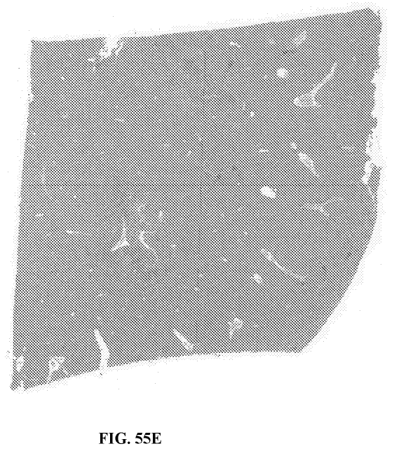

In vivo therapeutic efficacy in p53-null disseminated NSCLC model. To establish the experimental disseminated metastatic model, ˜1×10⁶p53-null H1299 cells in 100 μl of PBS were injected via tail vein into female athymic nude mice. Four weeks after the IV injection of tumor cells, mice were randomly divided into five groups (n=5), which received treatment with PBS, EGFP-mRNA NPs, everolimus, p53-mRNA NPs, or p53-mRNA NPs together with everolimus. The EGFP-mRNA NPs or p53-mRNA NPs were injected via tail vein at an mRNA dose of 750 μg/kg, whereas everolimus was orally administered at 5 mg/kg every three days for five rounds of treatment. The first treatment was performed at Day 0. On Day 15, all the mice were sacrificed, and one liver was randomly selected from each group for H&E staining. The liver section from each group was divided into four regions for calculation of the metastasis numbers (FIG. 55).

Immune response detection by the enzyme-linked immunosorbent assay (ELISA) assay. Female BALB/c mice (6 weeks old, n=3 per group) were intravenously injected with PBS, empty NPs, or p53-mRNANPs (750 μg mRNA/kg). Serum samples were collected after 24 hours of treatment. Representative cytokines (TNF-α, IFN-γ, IL-6, and IL-12) were detected by ELISA (PBL Biomedical Laboratories and BD Biosciences) according to the manufacturers' instructions.

In vivo toxicity evaluation. To evaluate in vivo toxicity, major organs were harvested at the end point of different tumor models (p53-null Hep3B liver xenograft tumor model, liver metastases of p53-null H1299 lung tumor model), followed by section and H&E staining to evaluate the histological differences. In addition, blood was drawn retro-orbitally and serum was isolated from p53-null Hep3B liver xenograft tumor model at the end of the efficacy experiment. Various parameters including ALT, AST, BUN, RBC, WBC, Hb, MCHC, MCH, HCT, and LY were tested to assess for toxicity.

Statistical analysis. Statistical analysis was carried out by GraphPad Prism 7 software to perform two-tailed t test or one-way ANOVA. All studies were performed at least in triplicate unless otherwise stated. Error bars indicate standard error of the mean (S.E.M). A P<0.05 value is considered statistically significant, where all statistically significant values shown in the figures are indicated as: *P<0.05, **P<0.01, and ***P<0.001.

Materials. L-Cystine dimethyl ester dihydrochloride ((H-Cys-OMe)₂. 2HCl), trimethylamine, cationic ethylenediamine core-poly(amidoamine) (PAMAM) generation 0 dendrimer (G0), and fatty acid dichloride were obtained from Sigma-Aldrich. DMPE-PEG with PEG molecular weight (MW) 2000 and DSPE-PEG with PEG molecular weight (MW) were purchased from Avanti Polar Lipids. Lipofectamine 2000 (Lip2k) was purchased from Invitrogen. EGFP-mRNA (modified with 5-methylcytidine and pseudouridine) and CleanCap Cyanine 5 FLuc mRNA (control Cy5-labeled Luc-mRNA) were purchased from TriLink Biotechnologies. Everolimus (RAD001) was obtained from Sigma-Aldrich. Primary antibodies used for western blot experiments and immunofluorescent and immunohistochemistry staining: anti-p53 (Santa Cruz Biotechnology, sc-126; 1:1,000 dilution), anti-BCL-2 (Abcam, ab59348; 1:1,000 dilution), anti-BAX (Cell Signaling Technology, #2774; 1:1,000 dilution), anti-PUMA (Santa Cruz Biotechnology, H-136; 1:1,000 dilution), anti-Cleaved Caspase3 (Cell Signaling Technology, #9661; 1:1,000 dilution), anti-Cleaved Caspase9 (Abcam, ab2324; 1:1,000 dilution), anti-p21 (Abcam, ab109520; 1:2,000 dilution), anti-Cyclin E1 (Abcam, ab3927; 1:2,000 dilution), anti-mTOR (Cell Signaling Technology, #2972; 1:1,000 dilution), anti-p-mTOR (Cell Signaling Technology, #5536; 1:1,000 dilution), anti-p-p70S6K (Cell Signaling Technology, #9205; 1:2,000 dilution), anti-p-4EBP1 (Cell Signaling Technology, #13443; 1:2,000 dilution), anti-LC3B (ABclonal, A7198; 1:1000 dilution), anti-SQSTM1/p62 (Abcam, ab56416; 1:2,000 dilution), anti-mouse p53 (Santa Cruz Biotechnology, sc-393031; 1:1000 dilution), anti-p-AMPKα (Cell Signaling Technology, #2535S; 1:1000 dilution), anti-p-ACCα (Cell Signaling Technology, #11818S; 1:1000 dilution), anti-TIGAR (Abcam, ab37910; 1:1000 dilution), anti-BECLIN1 (Cell Signaling Technology, #3495; 1:2000 dilution), anti-CD31 (Servicebio, GB11063-3; 1:250 dilution). Anti-GAPDH (Cell Signaling Technology, #5174; 1:2,000 dilution), anti-beta-Actin (Cell Signaling Technology; 1:2,000 dilution). Anti-rabbit and anti-mouse horseradish peroxidase (HRP)-conjugated secondary antibodies were obtained from Cell Signaling Technology. Secondary antibodies used for CLSM experiments included: Alexa Fluor 488 Goat-anti Rabbit IgG (Life Technologies, A-11034) and Alexa Fluor 647 Goat-anti Mouse IgG (Life Technologies, A-28181). The cationic lipid-like compound G0-C₁₄ was prepared through a ring opening reaction of 1,2 epoxytetradecane with G0 according to previously described methods (38). The hydrophobic PDSA polymers were synthesized by one-step polycondensation of (H-Cys-OMe)₂.2HCl and the fatty acid dichloride as described (41), and characterized with the ¹HNMR spectra using a Mercury VX-300 spectrometer at 400 MHz.

Cell lines. The p53-null human hepatocellular carcinoma (HCC) cell line Hep3B (Hep 3B2.1-7, ATCC #HB-8064) and the p53-null human non-small cell lung cancer (NSCLC) cell line H1299 (ATCC #CRL-5803) were purchased from American Type Culture Collection (ATCC). The p53-null murine hepatocellular carcinoma cell line RIL-175 was obtained from Prof Dan G. Duda's lab at Massachusetts General Hospital. Eagle's Minimum Essential Medium (EMEM; ATCC) was used to culture Hep3B cells, and Roswell Park Memorial Institute 1640 (RPMI-1640; ATCC) was used to maintain H1299 cells. Dulbecco's Modified Eagle's Medium (DMEM; ATCC) was used to culture RIL-175 cells. The cell culture medium was supplemented with 1% penicillin/streptomycin (Thermo-Fisher Scientific) and 10% fetal bovine serum (FBS; Gibco).

Synthesis of chemically modified p53-mRNA. The plasmid carrying the open-reading frame (ORF) of p53 with a T7 promoter was purchased from Addgene. Linearized DNA was digested with endonuclease HindIII/ApaI. Then, p53 ORF containing T7 promoter was amplified by PCR reaction and purified according to the manufacturer's protocol. For in vitro transcription (IVT), the MEGAscript T7 Transcription kit (Ambion) was used together with 1-2 μg purified PCR products (templates), 6 mM 3′-O-Me-m⁷G(5′)ppp(5′)G (anti-reverse cap analog, ARCA), 1.5 mM GTP, 7.5 mM 5-methyl-CTP, 7.5 mM ATP, and 7.5 mM pseudo-UTP (TriLink Biotechnologies). Reactions were conducted at 37° C. for 4 h and followed by DNase treatment. Afterwards, a poly(A) tailing kit (Ambion) was used for adding 3′ poly(A)-tails to IVT RNA transcripts. The p53-mRNA was purified by the MEGAclear kit (Ambion), followed by treatment with Antarctic Phosphatase (New England Biolab) at 37° C. for 30 min. Large amounts of p53-mRNA were custom-synthesized by TriLink Biotechnologies with 100-150 μg template containing p53 ORF and T7 promoter.

Electrostatic complexation between G0-C14 and mRNA. To evaluate the complexation of cationic compound G0-C14 with mRNA, we performed an electrophoresis study with E-Gel 2% agarose gels (Invitrogen) with naked p53-mRNA or p53-mRNA complexed with G0-C14 (weight ratios of G0-C14/mRNA: 0.1, 1, 5, 10, 15, and 20). To assess the stability of mRNA in organic solvent (DMF), naked mRNA was incubated with DMF for 30 min and then loaded into agarose gels. The gel was imaged under UV light, and the bands from all groups were analyzed.

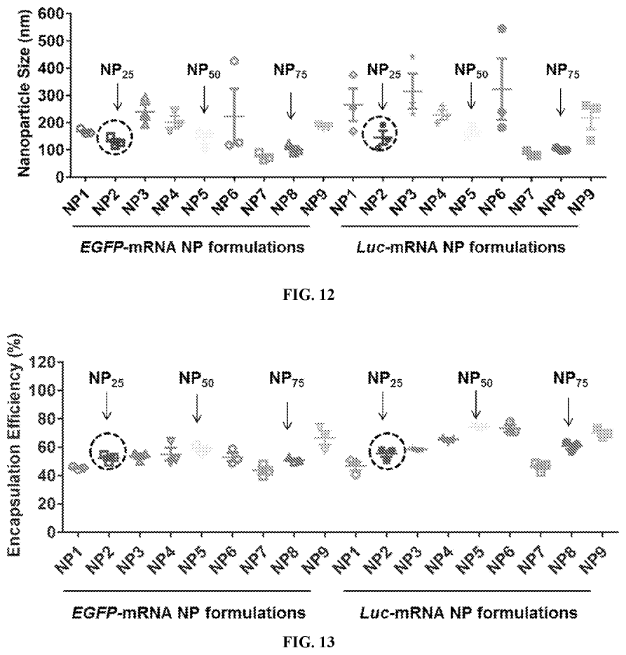

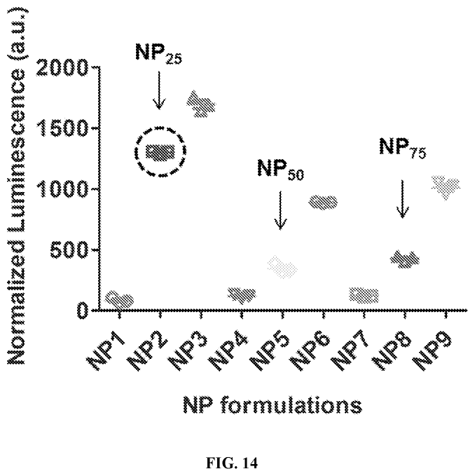

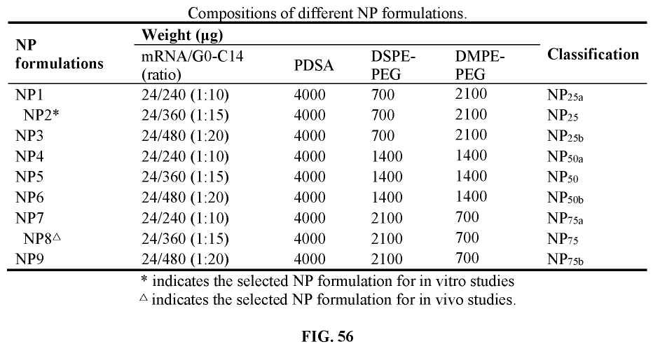

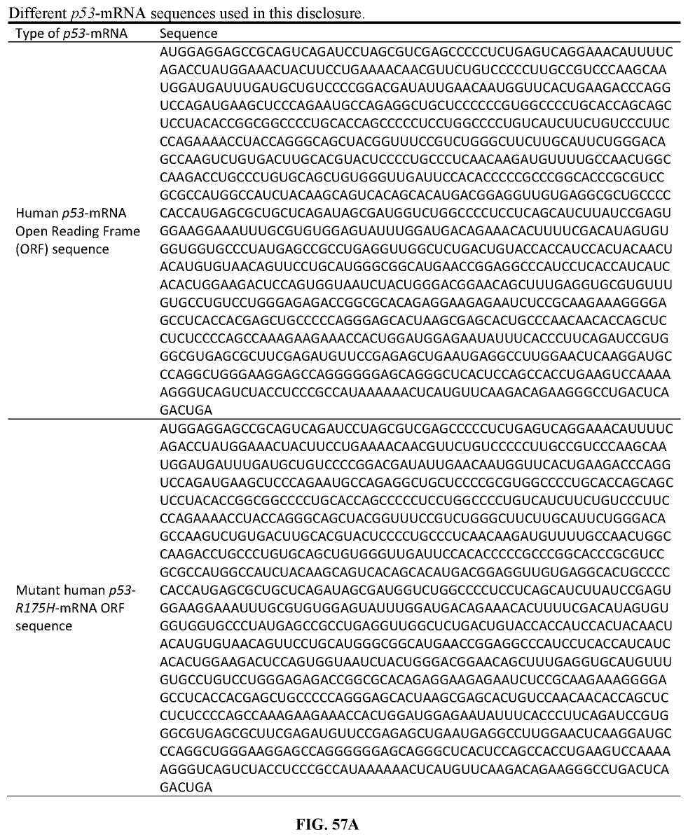

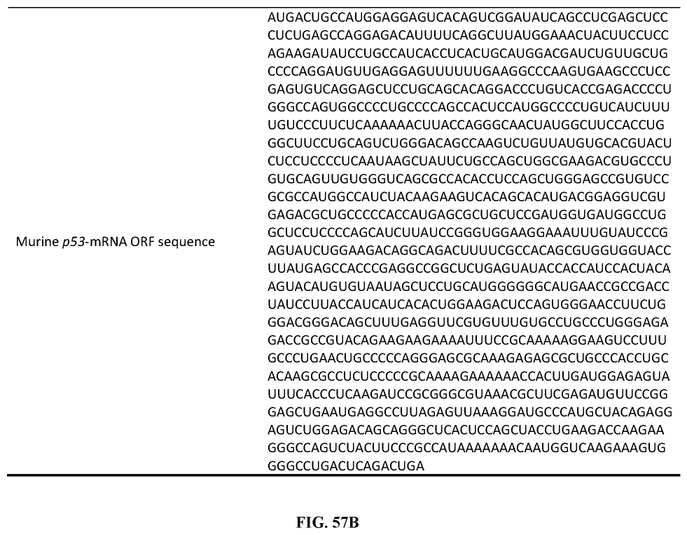

Formulation of the lipid-polymer hybrid mRNA NPs. A modified self-assembly method was adopted to prepare the mRNA-encapsulated lipid-polymer hybrid NPs. This method included the following steps: G0-C14, PDSA, and lipid-PEGs were dissolved separately in DMF to form a homogeneous solution at concentrations of 2.5 mg/ml, 20 mg/ml, and 20 mg/ml, respectively. 24 μg of mRNA (in 24 μl of water) and 360 μg of G0-C14 (in 144 μl of DMF) were mixed gently (at a G0-C14/mRNA weight ratio of 15) to enable the electrostatic complexation. Afterwards, 4 mg of PDSA polymers (in 200 μl of DMF) and 2.8 mg of hybrid lipid-PEGs (in 140 μl of DMF) were added to the mixture successively and further mixed together. The final mixture was added dropwise to 10 ml of DNase/RNase-free HyClone HyPure water (Molecular Biology Grade) under magnetic stirring (800 rpm) for 30 min. An ultrafiltration device (EMD Millipore, MWCO 100 kDa) was used to remove the organic solvent and free compounds in the formed NP dispersion via centrifugation. After washing 3 times with HyPure water, the mRNANPs were collected and dispersed in pH 7.4 PBS buffer for further use or stored at −80° C. We prepared the engineered mRNANPs with three different DSPE-PEG/DMPE-PEG ratios (NP₂₅: 25% of DSPE-PEG in lipid-PEG layer; NP₅₀: 50% of DSPE-PEG in lipid-PEG layer; NP₇₅: 75% of DSPE-PEG in lipid-PEG layer; w/w %). Two Cy5-labelled mRNAs with different molecular properties (EGFP-mRNA with a length of 996 nucleotides and Luc-mRNA with a length of 1,921 nucleotides) were chosen as model mRNAs to verify their potential effects on encapsulation and NP properties. As shown in FIG. 12, different compositions of G0-C14/PDSA/lipid-PEG (FIG. 56) changed NP size. Nevertheless, although the mRNA length of Luc-mRNA is ˜2-fold longer than that of EGFP-mRNA, its effect on NP size is not drastic. In addition, there was no obvious difference in mRNA encapsulation efficiency between the EGFP-mRNA NPs and the Luc-mRNA NPs for each formulation (FIG. 13). Considering the NP properties (especially the NP size) and the transfection efficacy (FIG. 14), we used 25% of DSPE-PEG (w/w %) in lipid-PEG layer (0.7 mg of DSPE-PEG and 2.1 mg of DMPE-PEG in 2.8 mg of hybrid lipid-PEGs; NP₂₅) for all in vitro studies.

Characterization of the synthetic mRNA NPs. We used dynamic light scattering (DLS, Brookhaven Instruments Corporation) to determine the size of the engineered mRNA NPs and their stability in PBS (containing 10% serum) at 37° C. over a span of 72 h. JEOL 1200EX-80 kV transmission electron microscope (TEM) was used to visualize the morphology of mRNA NPs. To test the mRNA encapsulation efficiency (EE %), Cy5-mRNA NPs were prepared according to the aforementioned method. In brief, 100 μl of dimethyl sulfoxide (DMSO) was used to treat 5 μl of the NP solution, and fluorescence intensity of Cy5-mRNA was tested by a Synergy HT multi-mode microplate reader. The amount of loaded mRNA in the engineered NPs was calculated to be ˜50% in this study.

Evaluation of the redox-responsive property of the mRNA NPs. The prepared Cy5-mRNA NPs were suspended in 1 ml of PBS (pH 7.4) containing DTT at the concentration of 10 mM. The morphology of the NPs was visualized by TEM after 2 or 4 hours of incubation. In addition, to verify the influence of redox on the mRNA release, Cy5-mRNA NPs were suspended in 1 ml of PBS and added in a Float-a-lyzer G2 dialysis device (MWCO=100 kDa, Spectrum), which was immersed in PBS or PBS containing DTT at different concentrations (1 mM and 10 mM) at 37° C. At different time points (1, 2, 4, 8, 12, and 24 h), 5 μl of the NP solution was taken and mixed with 100 μl of DMSO. The fluorescence intensity of Cy5-mRNA was tested by a microplate reader.

Cell viability and transfection efficiency of EGFP-mRNA NPs. The p53-null Hep3B cells or H1299 cells were plated in 96-well plates at a density of 3×10³ cells per well. After 24 hours of cell adherence, cells were transfected with EGFP-mRNA at various mRNA concentrations (0.102, 0.207, 0.415, or 0.830 μg/ml) for 24 hours, followed by the addition of 0.1 ml fresh complete medium and further incubation for another 24 hours to evaluate cell viability as well as the transfection efficiency. Lip2k was used as a positive control for transfection efficiency comparison with the NPs. Cell viability was tested by AlamarBlue assay, which is a non-toxic assay that can continuously check real-time cell proliferation through a microplate reader (TECAN, Infinite M200 Pro). Absorbance was examined by a 96-well SpectraMax plate reader (Molecular Devices) at 545 nm and 590 nm. To measure the transection efficiency, cells were treated with EGFP-mRNA by NPs or Lip2k for 24 hours, detached with 2.5% EDTAtrypsin, and collected in PBS solution, followed by evaluating GFP expression using flow cytometry (BD Biosystems). The percentages of EGFP-positive cells were calculated and analyzed by Flowjo software.

In vitro cell viability of p53-mRNA NPs or their combination with everolimus. The p53-null Hep3B or H1299 cells were plated in a 96-well plate at a density of 5×10³ cells per well. After 24 hours of cell adherence, cells were transfected with EGFP-mRNA NPs (control NPs), p53-mRNA NPs, everolimus, or p53-mRNANPs together with everolimus. The concentration of mRNA used was 0.415 μg/ml, whereas the concentration of everolimus was 32 nM in Hep3B cells or 16 nM in H1299 cells. After 24 hours of incubation followed by addition of 0.1 ml fresh complete medium for another 24 hours, the AlamarBlue cell viability assay mentioned above was used to verify the in vitro efficacy of p53-mRNANPs and their ability to sensitize cells to everolimus.

Colony formation assay. The cells' proliferation ability was measured by a soft agar colony formation assay. Cells were treated with p53-mRNA NPs or empty NPs for 48 hours. Then, cells were suspended in 0.36% agarose (Invitrogen) diluted in the complete medium, then reseeded into 6-well plates at low density (˜1000 cells per well) containing a 0.75% preformed layer of agarose and incubated for 2 weeks. The plates were then washed with PBS and fixed in 4% paraformaldehyde for 20 min and then stained with 0.005% crystal violet. The images of all the wells were scanned and analyzed.

Apoptosis and cell cycle detection in vitro. We used an FITC Annexin V/Propidium iodide (PI) apoptosis detection kit (BD Biosciences) to detect apoptosis. In brief, 1×10⁶ cells were seeded into 6-well plates. After attachment overnight, cells were treated with p53-mRNA NPs for 24 hours before being mixed with 1 ml fresh medium and continuing to culture for another 24 h. All the attached cells together with the floating cells in the medium were harvested, washed with PBS twice, and dispersed in 1× binding buffer solution (ice-cold) at a concentration of 1×10⁶ cells/ml. 5 μl of FITC Annexin V and 5 μl of PI were further mixed with 100 μl of the cell suspension. We then incubated the mixture at room temperature for 15 min in a dark environment and performed analysis using the FACS Calibur Flow Cytometer (BD Biosystems). Cells were incubated for 48 hours with empty NPs, naked p53-mRNA, or p53-mRNANPs washed in PBS and fixed with 70% ethanol overnight, then washed in PBS twice and incubated with PI for 30 minutes at 37° C.; cell-cycle fractions (percentage of cells with fractional DNA content in G1, S, and G2/M phases of the cycle) were estimated by flow cytometry and analyzed by Flowjo software.

Western blot assay. Cells or dissected tumors in each group were lysed in a lysis buffer (1 mM EDTA, 20 mM Tris-HCl pH 7.6, 140 mM NaCl, 1% aprotinin, 1% NP-40, 1 mM phenylmethylsulphonyl fluoride, and 1 mM sodium vanadate), and supplemented with protease inhibitor cocktail (Cell Signaling Technology). Protein concentration was detected by a bicinchoninic acid (BCA) Protein Assay Kit (Pierce). 25 μg of proteins were loaded on 6-12% precast gels (Invitrogen), and then transferred to Immobilon PVDF membranes (Bio-Rad, 162-0176 and 162-0177). The transferred membranes were blocked with 5% bovine serum albumin (BSA) in TBST (150 mM NaCl, 50 mM Tris-HCl at pH 7.4, and 0.1% Tween 20) for 1 hour at room temperature, and were further incubated with primary antibodies overnight at 4° C. The immunoreactive bands were detected with appropriate HRP-conjugated secondary antibodies. Band density was detected by enhanced chemiluminescence (ECL) detection system (Amersham/GE Healthcare).

Gene expression via quantitative real time polymerase chain reaction (qRT-PCR). qRT-PCR was used to quantify the expression of autophagy-related genes (DRAM1, ISG20L1, ULK1, ATG7, BECN1, ATG12, and SESN1) and p53 target gene TIGAR in Hep3B and H1299 cell lines. Total RNA was isolated using TRIzol (Invitrogen Life Technology) according to the protocol. RNA was quantitated by UV absorbance at 260 nm. cDNA was reverse-transcribed (RT) using a complementary DNA synthesis kit (Thermo Fisher Scientific, SuperScript III First-Strand Synthesis System). The qRT-PCR was performed in Real-Time PCR Detection instrument (Qiagen, Rotor Gene Q Series) using SYBR Green dye (Qiagen, Rotor-Gene SYBR Green PCR Kit). 25 μl of mixture containing 100 ng cDNA, 1 M primer dilution, and 12.5 μl 2×Roter-Gene SYBR Green PCR Master Mix was used in each PCR reaction. Fluorescence signal was recorded at the endpoint of each cycle during the cycles (denaturizing 15 sec at 95° C., annealing 45 sec at 60° C., and extension 20 sec at 72° C.). GAPDH was used as internal control gene for normalization. Relative gene expression was calculated by the comparative threshold cycle (CT), which represents the inverse of the amount of mRNA in the initial sample.

Design of the primers for qRT-PCR. Primers were designed via National Center for Biotechnology Information website. Primers were selected according to following criteria: (1) length between 18 and 24 bases; (2) melting temperature (Tm) between 57° C. and 60° C. (optimal Tm 58° C.); and (3) G+C content between 40% and 60% (optimal 50%). Primer sequences are listed in FIG. 57.

Immunofluorescent staining and TEM detection. Cells or tumor tissues were fixed with 4% paraformaldehyde (Electron Microscopy Sciences) at room temperature for 15 min, followed by permeabilization in 0.2% Triton X-100-PBS for 10 min. Samples were further incubated with PBS blocking buffer (containing 2% BSA, 2% normal goat serum, and 0.2% gelatin) at room temperature for 30 min. Afterwards, the samples were incubated with primary antibody overnight at 4° C., washed with PBS, and incubated in goat anti-rat-Alexa Fluor 647 (Molecular Probes) in blocking buffer (1:1000 dilution) at room temperature for 60 min. Stained samples were washed with PBS, nuclei were stained using Hoechst 33342 (Molecular Probes-Invitrogen, H1399, 1:2000 dilution in PBS), and the samples were mounted on slides with Prolong Gold antifade mounting medium (Life Technologies). For TEM detection, treated cells were washed and fixed by 2.5% glutaraldehyde solution (Sigma-Aldrich, G5882) overnight. After treatment with 1.5% osmium tetroxide, the samples were dehydrated in graded ethanol, and then embedded in 812 resin (Ted Pella, 18109). Thin sections were sliced and poststained with 2% uranyl acetate, then imaged with the TECNAI TEM (Philips).

Quantification of GFP-LC3B puncta. For GFP-LC3B autophagy assays, prepackaged viral particles expressing recombinant GFP-LC3B (LentiBrite GFP-LC3B Lentiviral Biosensor; Millipore, 17-10193) were used to generate GFP-LC3B stable cell lines. Then, GFP-LC3B stable cells were treated with everolimus or p53-mRNANPs and incubated for 24 hours at 37° C. A confocal fluorescence microscope was used to observe the fluorescence of GFP-LC3B. To quantify the extent of autophagy, cells showing accumulation of GFP-LC3B in vacuoles or dots were counted. Cells showing several intense punctate GFP-LC3B aggregates but no nuclear GFP-LC3B were defined as autophagic, whereas those presenting diffuse distributions of GFP-LC3B positive puncta (green) in both the cytoplasm and nucleus were considered as non-autophagic.

Immunohistochemistry (IHC) staining. Samples were obtained from different tumor models (p53-null Hep3B liver xenograft tumor model and liver metastases of p53-null H1299 lung tumor model). Sections were fixed in 4% buffered formaldehyde solution for 24 hours and embedded in paraffin, then sectioned into thin slices (5 μm thick) to be further deparaffinized, rehydrated in a graded ethanol series, and washed in distilled water. To retrieve the antigen, tumor tissue sections were incubated in 10 mM citrate buffer (pH=6) for 30 min, washed in PBS, and immersed in 0.3% hydrogen peroxide (H₂02) for 20 min, then incubated in blocking buffer (5% normal goat serum and 1% BSA) for 60 min. Tissue sections were then incubated with primary antibodies (PBS solution supplemented with 0.3% Triton X-100) at 4° C. overnight in a humid chamber. After being rinsed with PBS, the samples were incubated with biotinylated secondary antibody at room temperature for 30 min, washed again with PBS, followed by incubation with the avidin-biotin-horseradish peroxidase complex (ABC kit, Vector Laboratories, Inc). After being washed again, stains were processed with the diaminobenzidine peroxidase substrate kit (Impact DAB, Vector Laboratories, Inc) for 3 min. Sections were evaluated under a Leica Microsystem microscope after being counterstained with hematoxylin (Sigma), dehydrated, and mounted.

TUNEL apoptosis assay. Apoptotic cells in tumor tissues were measured by TUNEL staining using a detection kit (In Situ Cell Death Detection Kit, TMR red; Roche, #12-156-792-910) according to the manufacturer's protocol. Tumor sections were extracted and fixed in formalin, embedded in paraffin, and sectioned at a thickness of 5 μm. DAPI stain was used to assess total cell number. TUNEL-positive cells had a pyknotic nucleus with red fluorescent staining, representative of apoptosis. Images of the sections were taken by a fluorescence microscope (Olympus).

Combination index (CI) calculation. A reported method was used to calculate the CI value (51, 52). Briefly, the expected value of combination effect (Vexp) between treatment of everolimus and p53-mRNA NPs was calculated using formula (1) as follows:

$$Vexp = ( \frac{V 1}{Vctrl} ) \times ( \frac{V 2}{Vctrl} ) \times Vctrl \qquad ( 1 )$$

where Vctrl is the observed value of control group (cell viability for in vitro studies and tumor volume for in vivo studies), VI is the observed value of everolimus treatment, and V2 is the observed value of p53-mRNA NPs treatment. The CI was then calculated using formula (2) as follows:

$$CI = \frac{Vexp}{Vobs} \qquad ( 2 )$$

where Vobs is the observed value of combination effect between treatments with everolimus and p53-mRNA NPs. The combination effect was evaluated by the value of CI, with CI>1 indicating a synergistic effect.

### Example 1—Engineering and Characterization of Synthetic mRNA NPs

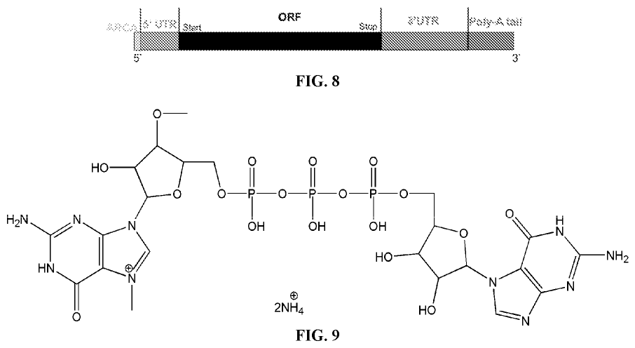

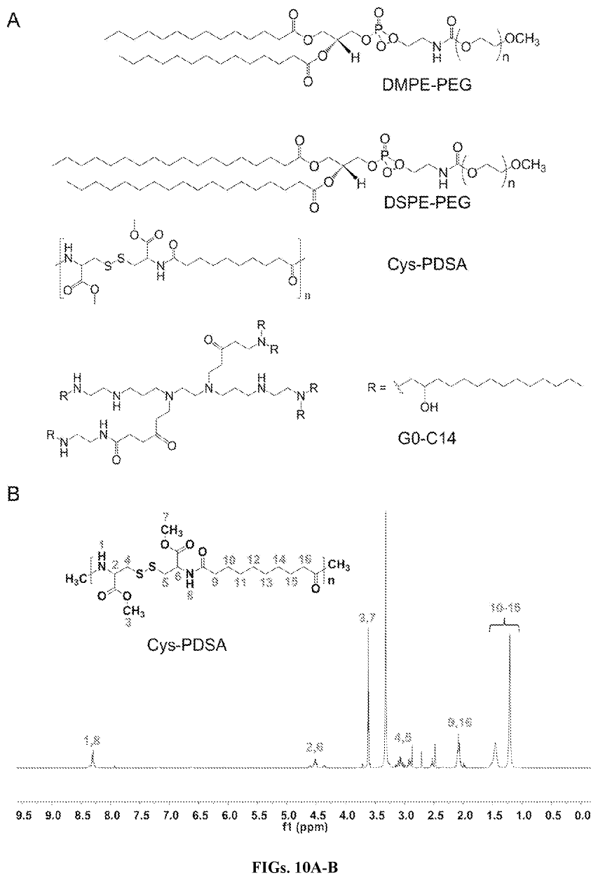

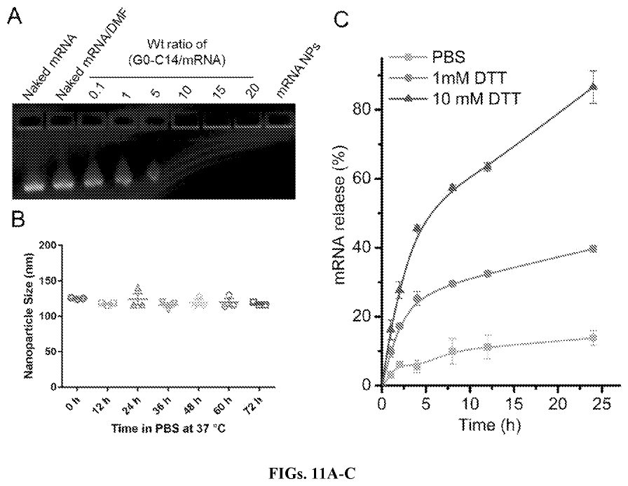

In vitro transcription (IVT) was used to synthesize enhanced green fluorescent protein (EGFP) mRNA and p53 mRNA (FIG. 7A). The 5′ terminal of mRNA was designed with an untranslated region (UTR) to enhance the translational initiation of the mRNA (FIG. 8). Anti-Reverse Cap Analog (ARCA) capping of 3′-O-Me-m⁷G(5′)ppp(5′)G (FIG. 9) and enzymatic polyadenylation were further used to modify the mRNA to increase its stability and translation efficiency. To reduce mRNA immunostimulation, 5-methylcytidine-5′-triphosphate (5-Methyl-CTP) and pseudouridine-5′-triphosphate (Pseudo-UTP) were used to replace regular CTP and UTP (36, 37). A robust self-assembly approach (38-40) was used to engineer lipid-polymer hybrid NPs for effective loading of the chemically modified mRNA, by using a cationic lipid-like molecule G0-C14, a hydrophobic redox-responsive cysteine-based poly(disulfide amide) (PDSA), and two lipid-poly(ethylene glycol) (lipid-PEG) compounds (FIG. 10). The cationic G0-C14 was used for mRNA complexation and to facilitate its cytosolic transport (40), and the PDSA was chosen to form a stable NP core under normal physiological conditions, while providing a rapid triggered release of payloads in tumor cells with high intracellular concentration of glutathione (GSH) (41-43). Both 1,2-dimyristoyl-sn-glycero-3-phosphoethanolamine-N-[methoxy(polyethylene glycol)] (DMPE-PEG) and 1,2-distearoyl-sn-glycero-3-phosphoethanolamine-N-[methoxy(polyethylene glycol)] (DSPE-PEG) were coated onto the surface of the hybrid NPs to simultaneously achieve a relatively long circulation time and high tumor cell uptake through a de-PEGylation effect (39). As shown in FIG. 11A, mRNA could be effectively condensed with G0-C14 at a weight ratio (G0-C14/mRNA w/w %) of 10 or above, with no effect of the dimethylformamide (DMF) solvent used for NP formulation on the integrity of mRNA. The redox-responsive hybrid NPs were prepared at the G0-C14/mRNA weight ratio of 15, and the engineered mRNANPs showed an average size of ˜125 nm and were stable in physiological conditions (FIG. 11B). As characterized by transmission electron microscopy (TEM) (FIG. 1A), the solid PDSA polymer core contributed to the formation of a rigid and stable nanostructure in pH 7.4 phosphate buffered saline (PBS), while efficiently responding to dithiothreitol (DTT, a reductive agent) by rapid disassembly of the NPs for release of mRNA (FIG. 11C). The redox-triggered sufficient release of payloads could potentially contribute to more effective therapeutic activities (41-47). The evaluation and selection of mRNANP formulations are provided in figs. 12-14 and 56.

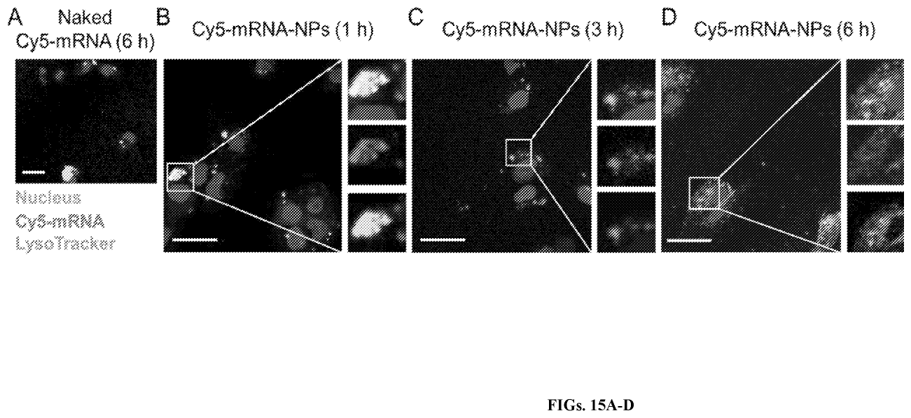

The cytosolic delivery of mRNA was examined using the engineered NPs in vitro. As shown in FIG. 1B and FIG. 15, the NPs could effectively transport Cy5-labeled mRNA into the cytoplasm in a time-dependent manner. Most of the internalized mRNA NPs first co-localized with LysoTracker Green at 1 hour. After 3 hours of incubation, some of Cy5-labeled mRNA entered the cytoplasm, and at 6 hours after incubation, a large amount of them escaped from endosomes and diffused into the cytoplasm. In comparison, naked mRNA could not readily enter the cells after 6 hours of incubation. The efficient cytosolic delivery of mRNA with the hybrid NPs could be observed in both p53-null HCC (Hep3B) and NSCLC (H1299) cells.

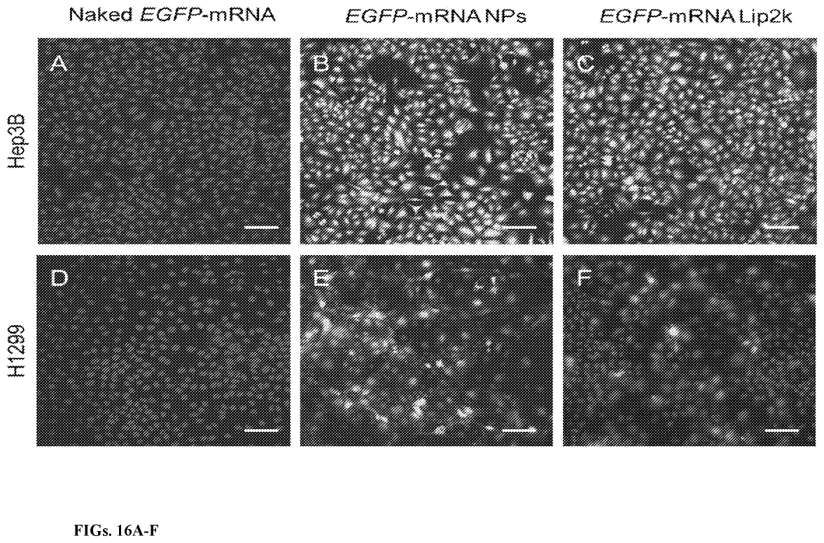

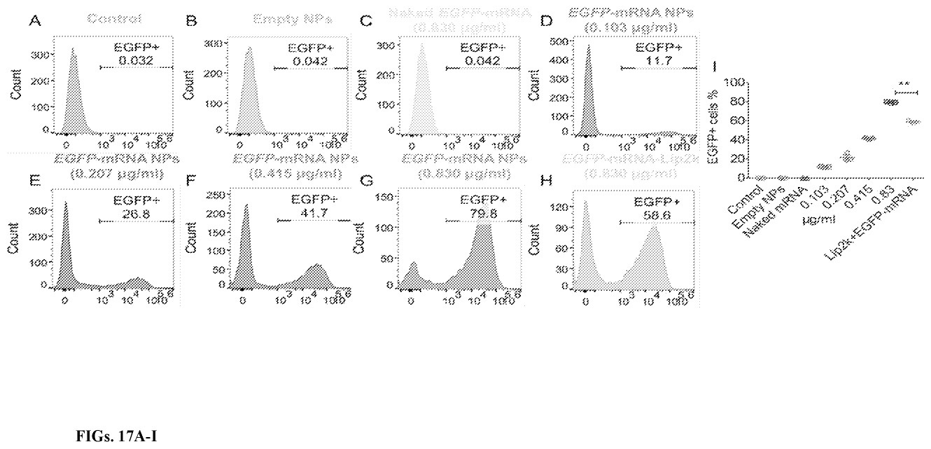

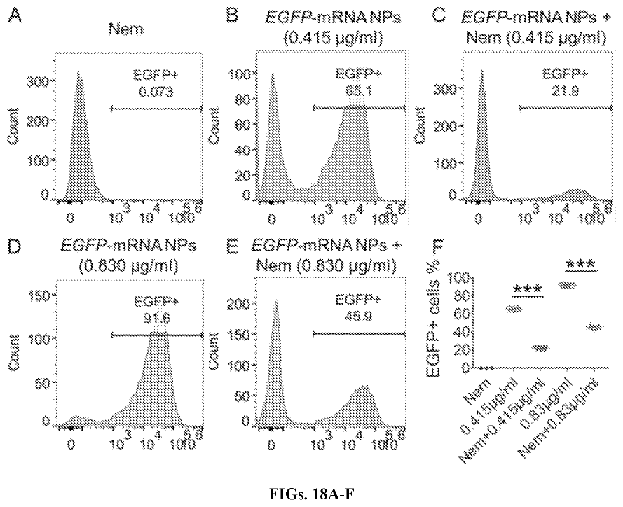

To further check the transfection efficacy in vitro, EGFP-mRNA was chosen as a model mRNA. The high transfection efficiency of the EGFP-mRNA NPs can be directly visualized by confocal laser scanning microscopy (CLSM), with considerable green fluorescence detected in both NP-transfected and commercial transfection agent lipofectamine 2000 (Lip2k)-transfected cells (FIG. 16). To quantitatively analyze mRNA transfection, EGFP expression in Hep3B and H1299 cells was measured by flow cytometry (FIG. 1C, D and FIG. 17). The EGFP expression showed a dose-dependent increase (EGFP-mRNA concentration from 0.103 to 0.830 μg/ml). Moreover, the percentage of EGFP-positive cells was significantly higher for the NP-transfected cells than for Lip2k-transfected cells at the concentration of 0.830 μg/ml (P<0.01), indicating a better transfection efficacy with the NP-mediated strategy in both Hep3B and H1299 cells. Notably, when using N-ethylmaleimide (Nem) to quench intracellular GSH, we noticed a marked decrease of EGFP expression by the mRNA NPs (FIG. 18), indicating that the redox-triggered mRNA release within the tumor cells may lead to better bioactivity. Moreover, no obvious in vitro cytotoxicity was observed in Hep3B and H1299 cells with all the tested concentrations of EGFP-mRNA NPs via AlamarBlue assay (FIG. 13). These results suggested the potential of the engineered hybrid NPs for synthetic mRNA delivery to restore tumor suppressor p53 in p53-null tumor cells.

### Example 2—Hybrid mRNA NP-Mediated p53 Restoration in p53-Null HCC and NSCLC Cells

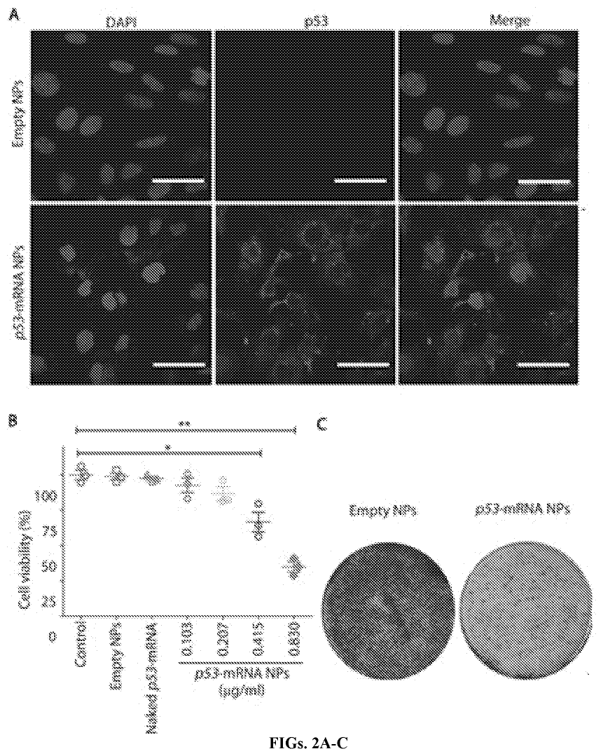

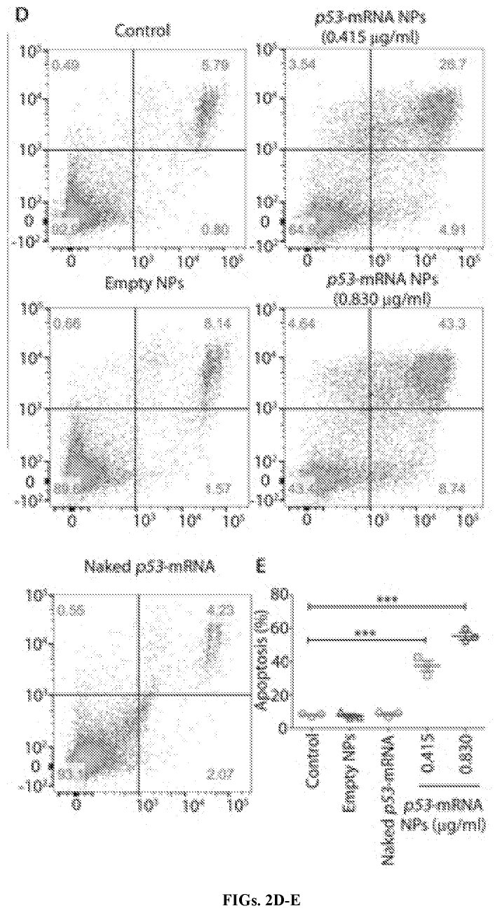

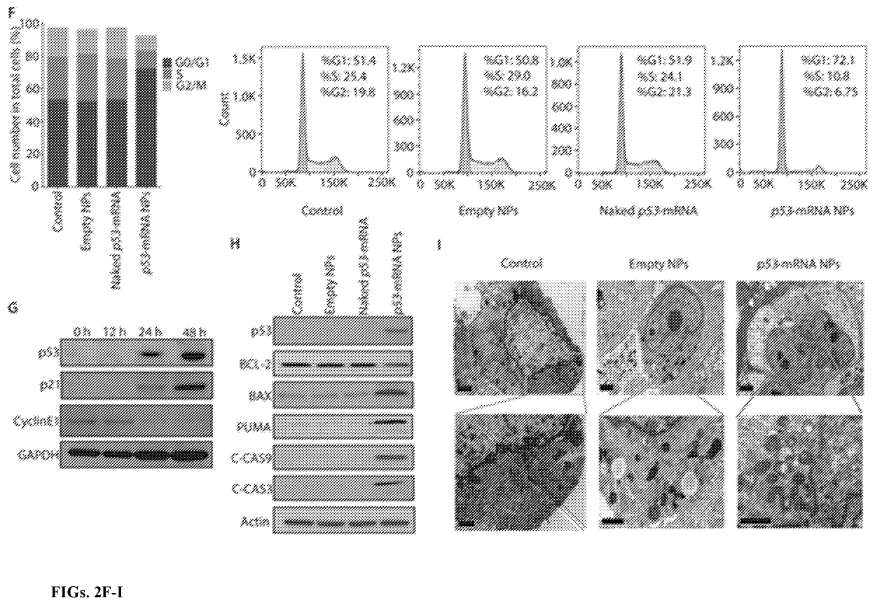

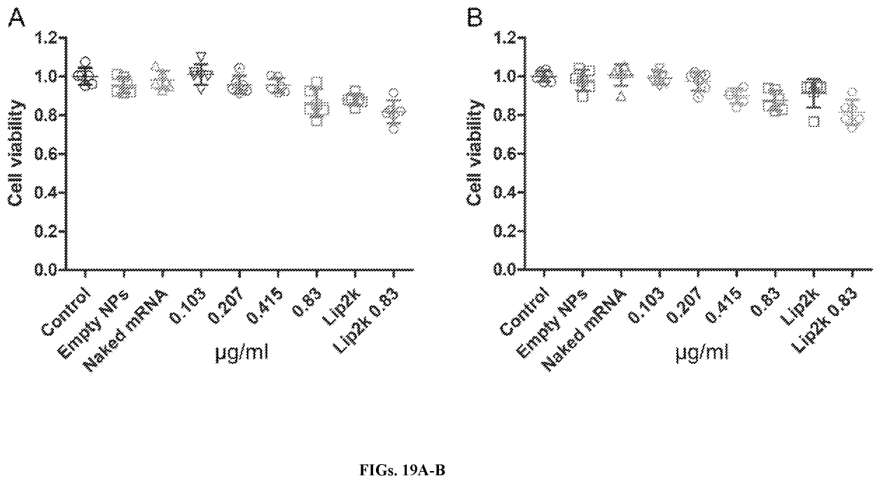

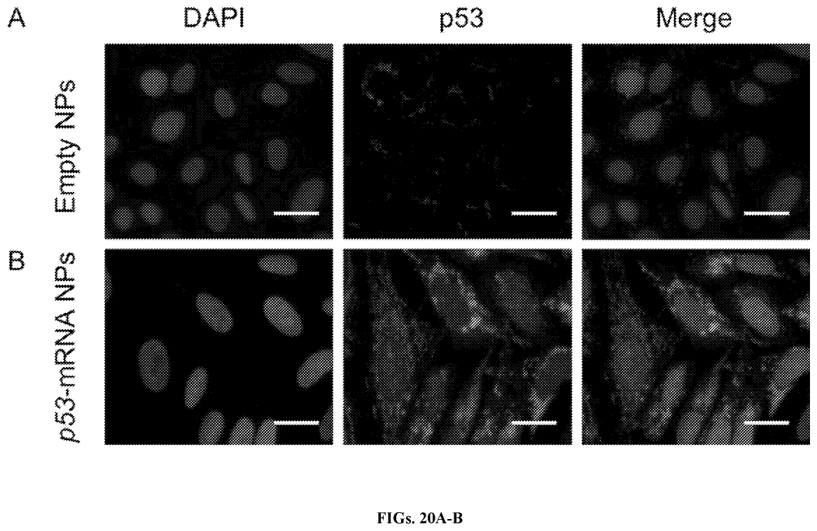

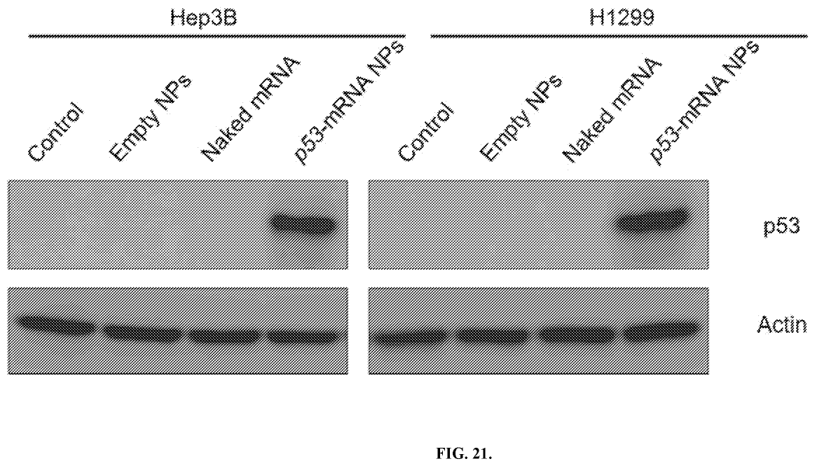

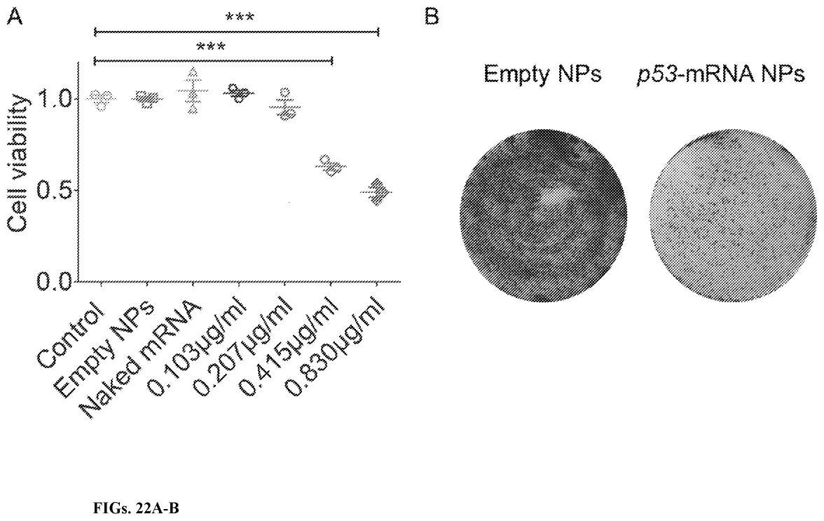

To examine the mRNANP strategy for restoration of tumor suppressor p53 in p53-null Hep3B and H1299 cells, immunofluorescence (IF) staining and western blot (WB) were performed to check the p53 protein expression in both cell lines after treatment with p53-mRNANPs. The IF results showed that p53 proteins were mainly expressed in the cytoplasm of both cell lines (FIG. 2A and FIG. 20). WB results also demonstrated that the expression of p53 protein was obviously increased in both cells after NP treatment (FIG. 21). Next, we tested whether the p53-mRNANPs could restore the suppressing function of p53 inp53-null tumor cells. After incubation with different doses of p53-mRNANPs, strong cytotoxicity was observed in a dose-dependent manner in Hep3B (FIG. 2B) and H1299 (FIG. 22A) cells. Colony formation was also dramatically inhibited in both cells treated with p53-mRNA NPs vs. empty NPs, further demonstrating p53 restoration-mediated anti-tumor activities (FIG. 2C and FIG. 22B). Meanwhile, apoptosis was measured using the annexin V (AnnV) and propidium iodide (PI) co-staining method followed by flow cytometry analysis. As can be seen in FIG. 2D, 2E and FIG. 23, cell apoptosis greatly increased after treatment with p53-mRNANPs at the concentrations of 0.415 and 0.830 μg/ml in Hep3B and H1299 cells, whereas empty NPs and naked mRNA did not induce apoptosis.

In addition, the cell-cycle phase distribution was studied upon treatment with p53-mRNANPs in Hep3B and H1299 cells. FIG. 2F showed that Hep3B cells treated with p53-mRNANPs had a larger G1 population (72.1%) compared with ˜50% in the control, empty NPs, or naked mRNA groups. Concomitant decreases were observed in S and G2 phases after p53-mRNA NP treatment, compared with the control, empty NPs, or naked mRNA groups. Similar results were observed in H1299 cells (FIG. 24), suggesting that p53 restoration could effectively induce G1-phase cell cycle arrest to inhibit cell proliferation. The signaling pathways involved in cell cycle regulation was also examined by evaluating the cell cycle-related proteins in Hep3B cells (FIG. 2G). The restoration of p53 functions by mRNA NPs resulted in the upregulation of p21 and the downregulation of Cyclin E1 from 12 to 48 hours, and it blocked the cell cycle at the G1 phase.

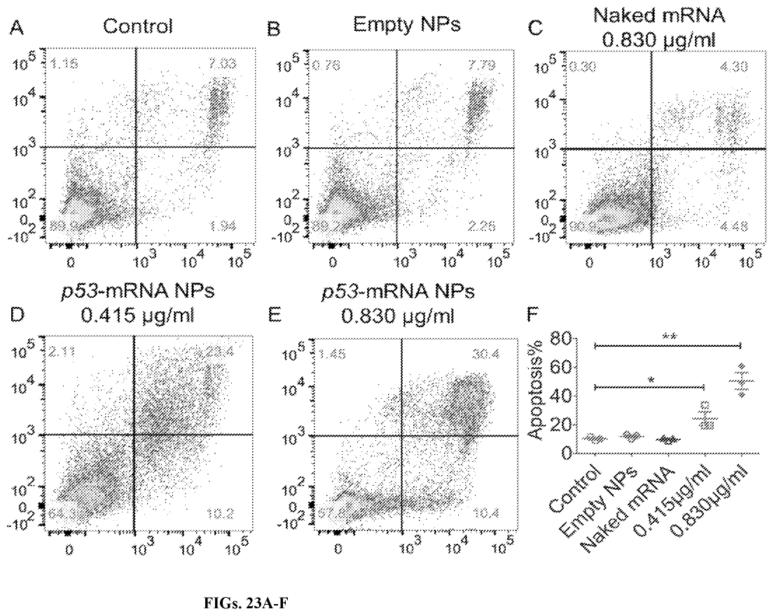

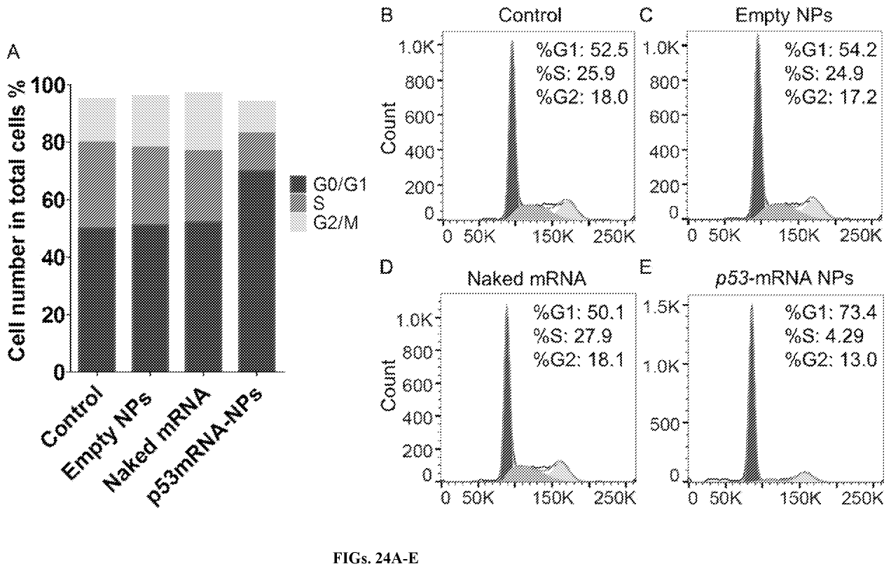

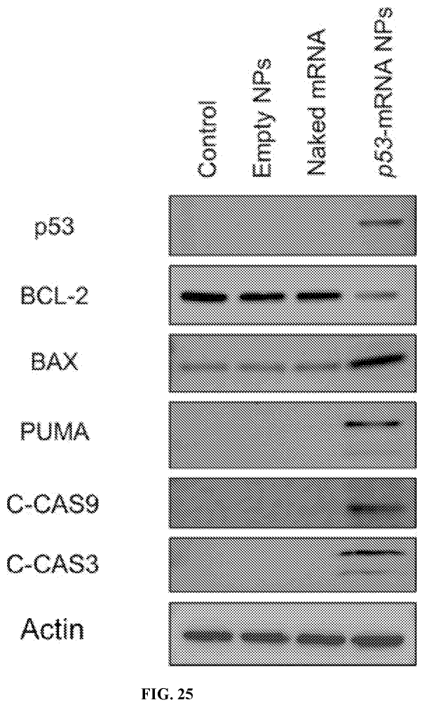

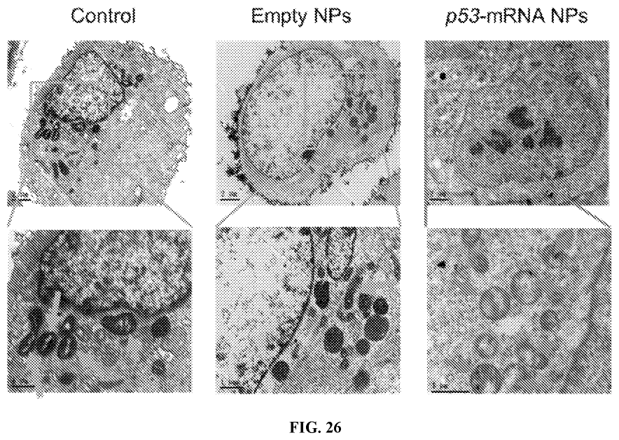

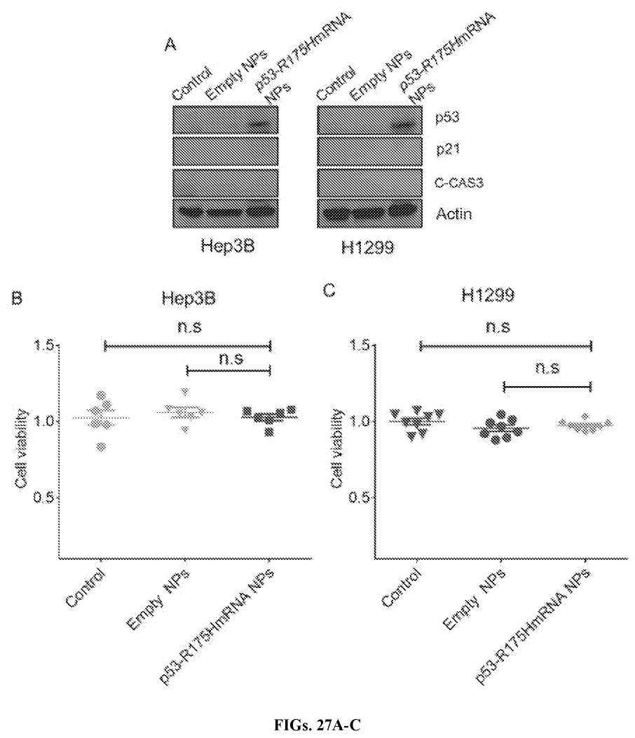

To further assess the in vitro anti-tumor mechanisms of the p53-mRNANPs in p53-null Hep3B and H1299 cells, WB studies were performed to verify the effects of p53 on the apoptosis pathway. As shown in FIG. 2H and FIG. 25, p53-mRNA NPs efficiently activated PUMA to initiate the cleaved caspase9 (C-CAS9)- and cleaved caspase3 (C-CAS3)-induced apoptosis pathway. This pathway was further confirmed through TEM analysis of mitochondrial morphology change, which is usually a common phenomenon for this apoptosis pathway (48, 49). Consistent with the WB results, increased numbers of swollen mitochondria (red arrows) were observed in the cytoplasm of Hep3B and H1299 cells after treatment with p53-mRNA NPs (FIG. 21 and FIG. S20), as compared to the control and empty NPs groups. These results indicated that p53 restoration by mRNA NPs within the present claims causes mitochondrial depolarization and swelling, further confirming the initiation of cellular apoptosis. Moreover, a mutant p53-R175H-mRNA (FIG. 57) was designed and tested as another control mRNA. As shown in FIG. 27, treatment with p53-R175H-mRNA NPs induced the expression of mutant p53 in both Hep3B and H1299 cells. However, neither p21 nor C-CAS3 was detected after NP treatment. The expression of the mutant p53 also did not cause cytotoxicity.

### Example 3—p53 Restoration Sensitizes p53-Null HCC and NSCLC Cells to mTOR Inhibitor Everolimus

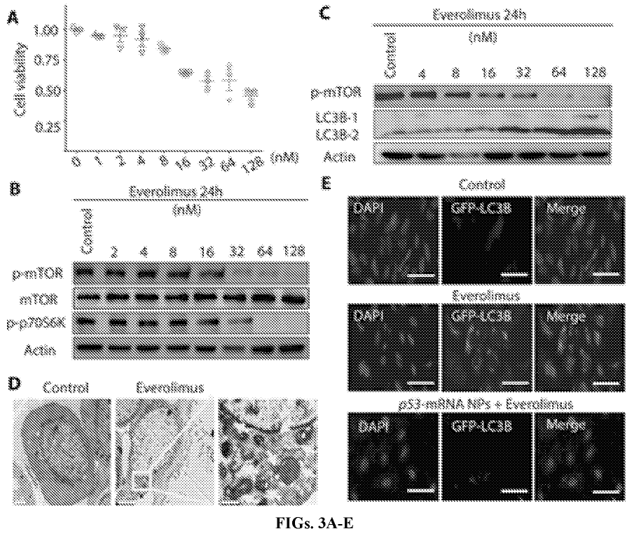

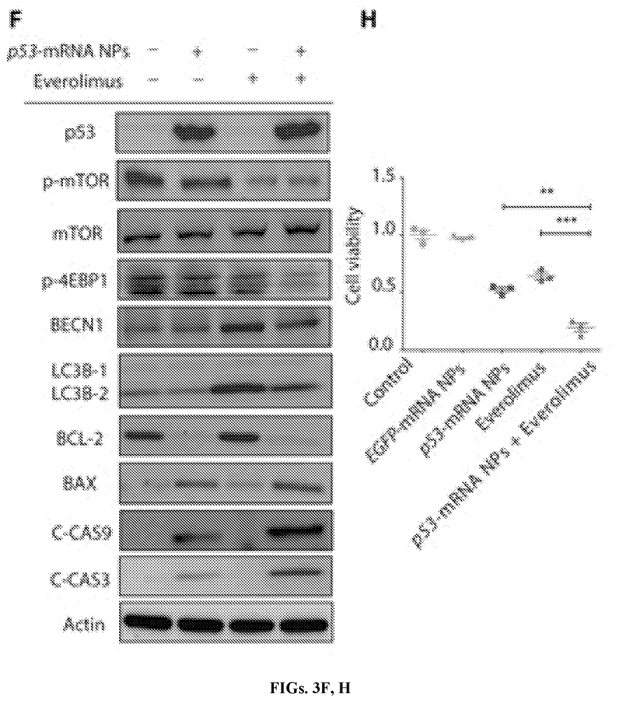

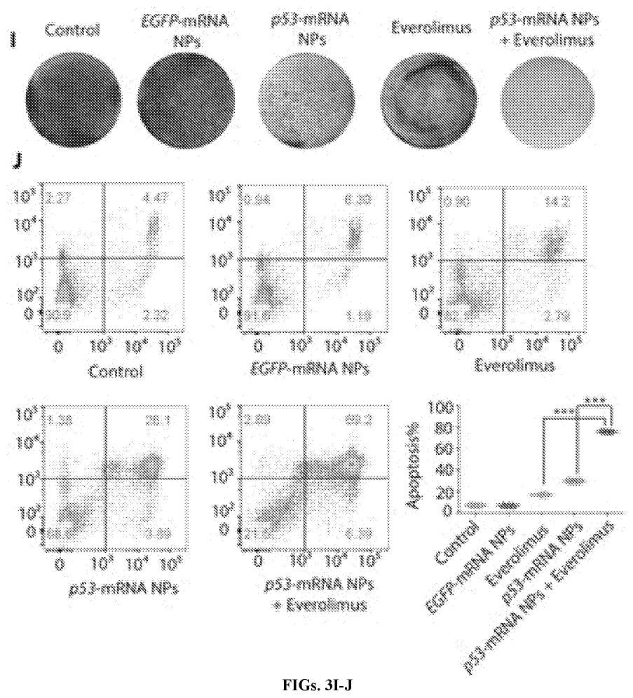

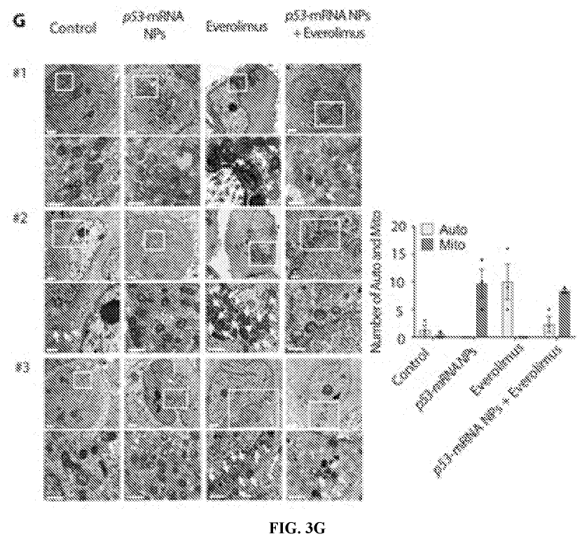

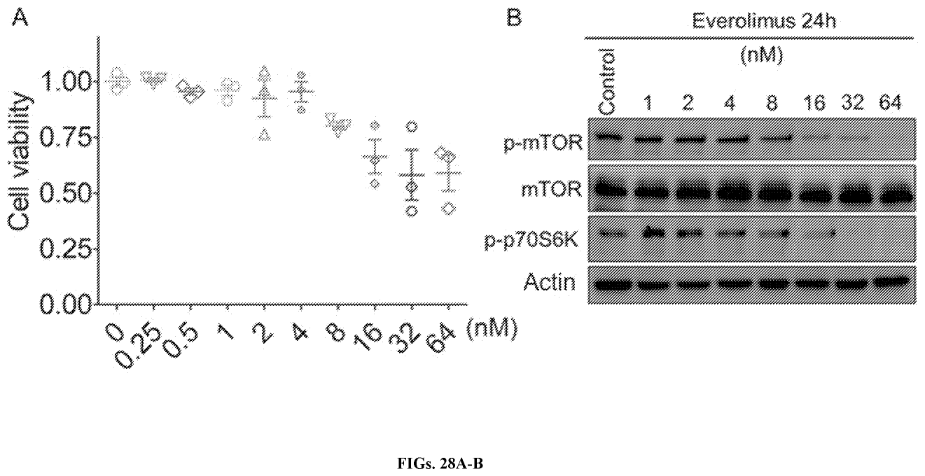

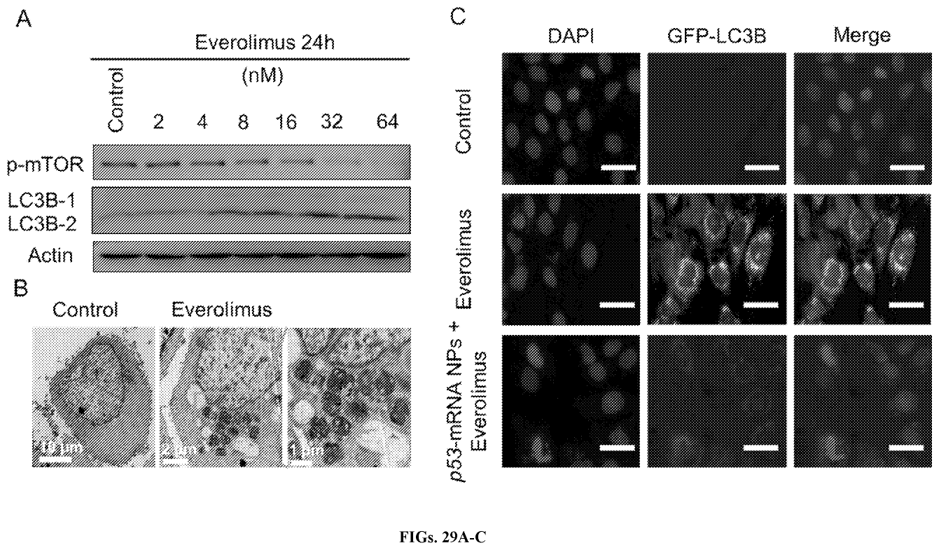

To examine the effects of p53 restoration on everolimus activity, the cytotoxicity of this mTOR inhibitor was measured inp53-null Hep3B and H1299 cells and explored its effect on the mTOR pathway. FIG. 3A and FIG. 28 indicate relative insensitivity of Hep3B and H1299 to everolimus, with over 50% of cells still alive at 64 nM. More importantly, although the mTOR pathway targets (p-mTOR and p-p70S6K) were substantially blocked by increasing everolimus concentrations (FIG. 3B and FIG. 28B), there was no significant decrease in cell viability. The effect of everolimus on the autophagy pathway was then examined. According to the method previously reported (50), the extent of autophagy can be measured by the ratio of LC3B-2/actin on WB. With the increase of everolimus concentration, upregulation of LC3B-2 and higher LC3B-2/actin ratios were observed by WB (FIG. 3C). The increased number of autophagosomes by TEM and increased fluorescence intensity of GFP-LC3B by CLSM were also consistent with the activation of autophagy by everolimus in Hep3B and H1299 cells (FIG. 3D-E and FIG. 29).

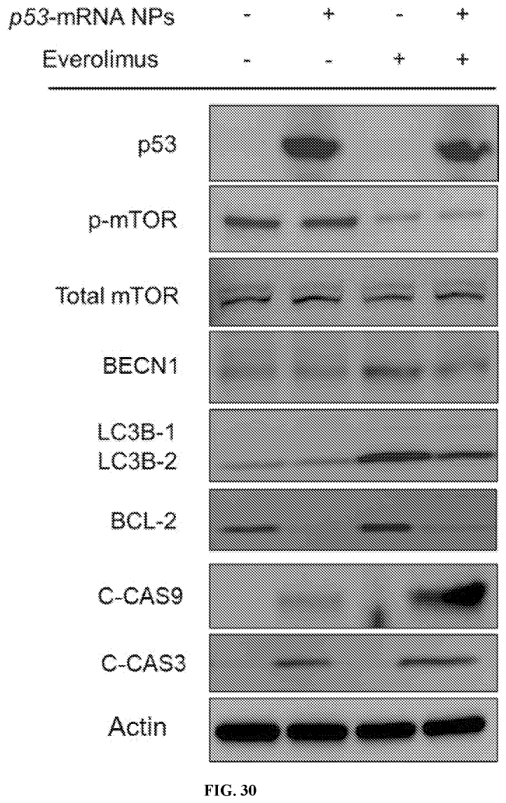

Next, it was examined whether the p53-mRNA NPs could inhibit the autophagy induced by everolimus. Both the CLSM and WB results in FIGS. 3E and 3F demonstrated that treatment with p53-mRNA NPs drastically reduced autophagy activation in p53-null Hep3B cells. The reduced number of autophagosomes (yellow arrows) was also observed in the “p53-mRNA NPs+everolimus” group as compared to the everolimus alone group by TEM (FIG. 3G). Moreover, it was tested whether, in the presence of everolimus, the p53-mRNA NPs could still restore the apoptotic pathway in Hep3B cells, similar to those shown in FIG. 2. As can be seen in FIGS. 3F and 3G, the upregulated expression of C-CAS3/9 and increased number of swollen mitochondria (red arrows) suggested the successful activation of the apoptotic pathway after treatment with p53-mRNA NPs. Similar results could also be observed inp53-null H1299 cells (figs. 29C, 30, and S31).

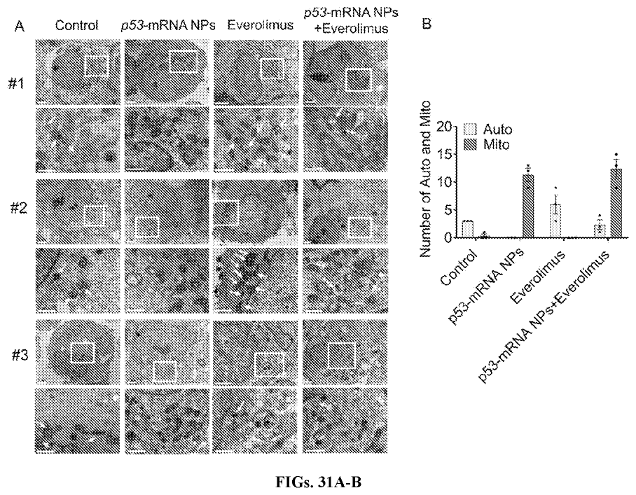

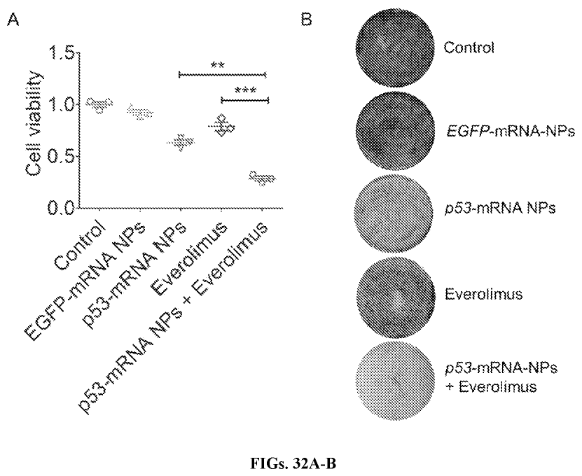

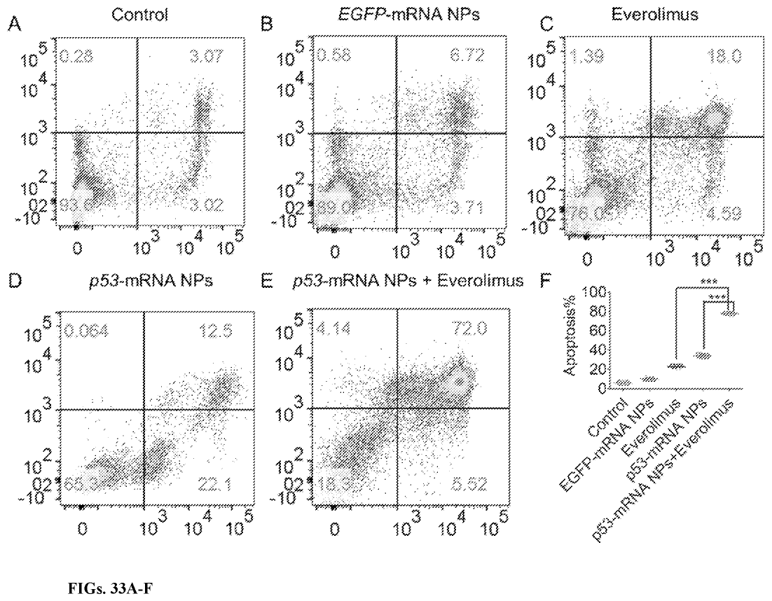

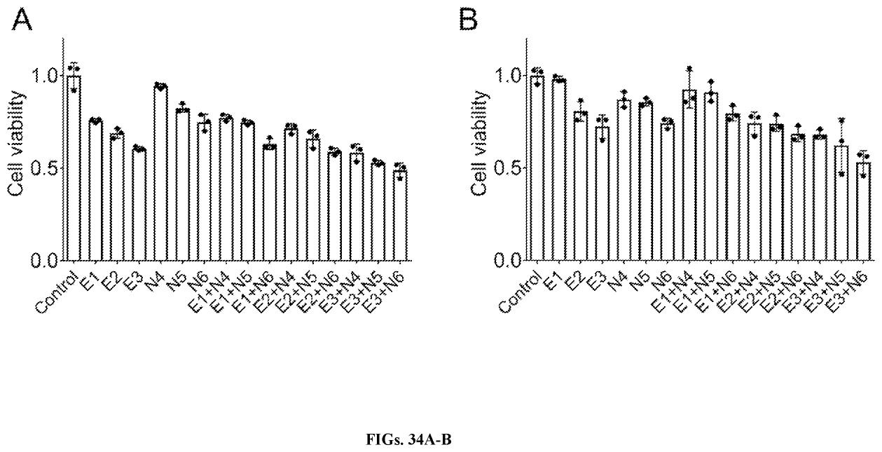

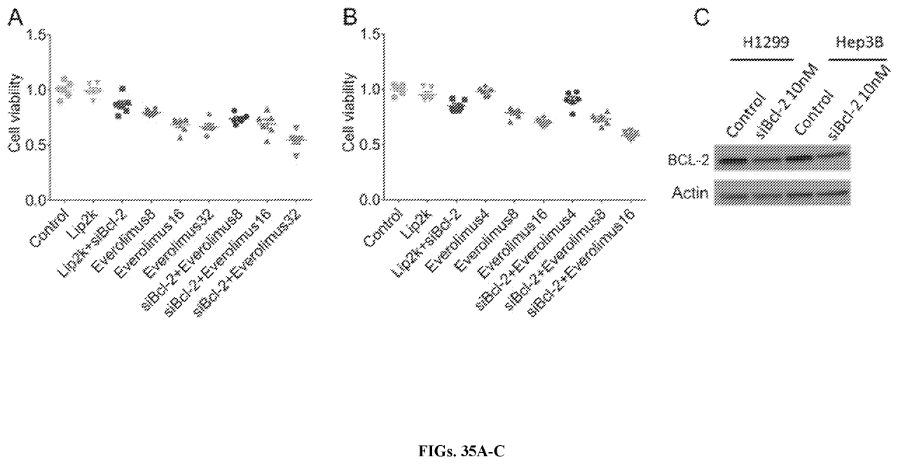

Motivated by the results showing inhibition of the autophagy pathway and activation of the apoptotic pathway, it was next determined whether the p53-mRNA NPs could sensitize Hep3B and H1299 cells to everolimus. As measured by AlamarBlue assay (FIG. 3H and FIG. 32A), everolimus showed a moderate therapeutic effect (with ˜70% viability in Hep3B cells and over 80% viability in H1299 cells), whereas co-treatment with everolimus and p53-mRNANPs showed strong in vitro anti-tumor effects in both cell lines (with ˜19% viability in Hep3B cells and ˜14% viability in H1299 cells). The EGFP-mRNA NPs were used as control NPs and did not show cytotoxicity. The combination index (CI) was also calculated using a reported method (51, 52) to assess whether there was a synergistic effect of the combination treatment. The CI value of “p53-mRNA NPs+everolimus” treatment was 1.71 in Hep3B cells and 1.74 in H1299 cells, indicating the presence of a synergistic effect (CI>1) in both cell lines. The colony formation assay also showed a marked reduction in live cells after co-treatment with p53-mRNA NPs and everolimus (FIG. 31 and FIG. 32B). Consistent with the above, flow cytometry analysis of apoptosis demonstrated that everolimus induced moderate apoptotic cell death, whereas co-treatment with everolimus and p53-mRNA NPs effectively augmented apoptosis (FIG. 3J and FIG. 33). To investigate the synergistic effect, we tested whether the inhibition of BCL-2 may also contribute to the improvement in everolimus sensitivity, as previously reported with small cell lung cancer (SCLC) H-510 cells (14). Two strategies (small molecular inhibitor venetoclax and siRNA) were used to target BCL-2 and combine with everolimus. Both approaches showed moderate combinatorial anti-tumor effect from BCL-2 inhibition together with high-dose everolimus (FIGS. 34 and 35), indicating that BCL-2 inhibition may not contribute to the improved everolimus sensitivity in p53-null Hep3B or H1299 cells. These results suggest that the synthetic mRNA NP-mediated p53 restoration can sensitize p53-null HCC and NSCLC cells to everolimus, presumably by inhibiting the activation of pro-survival autophagy.

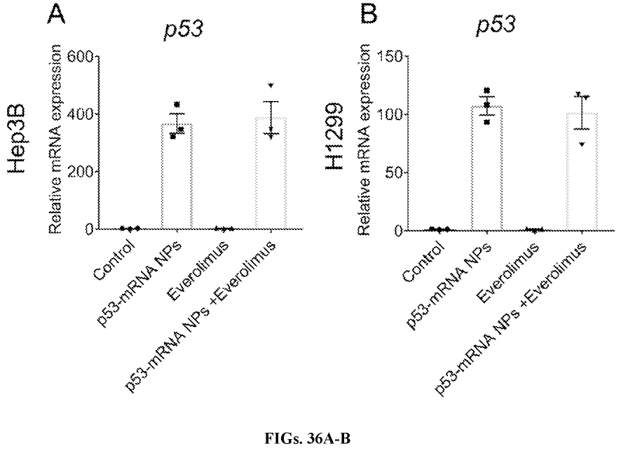

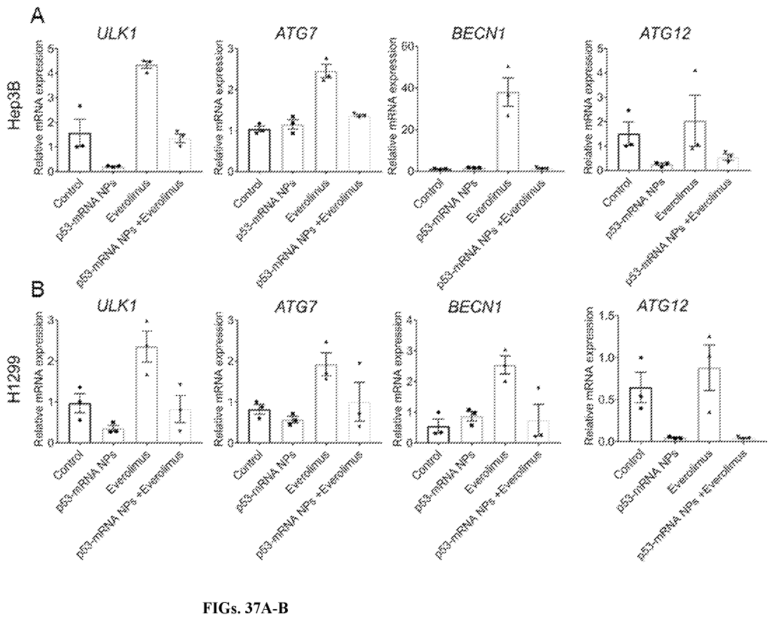

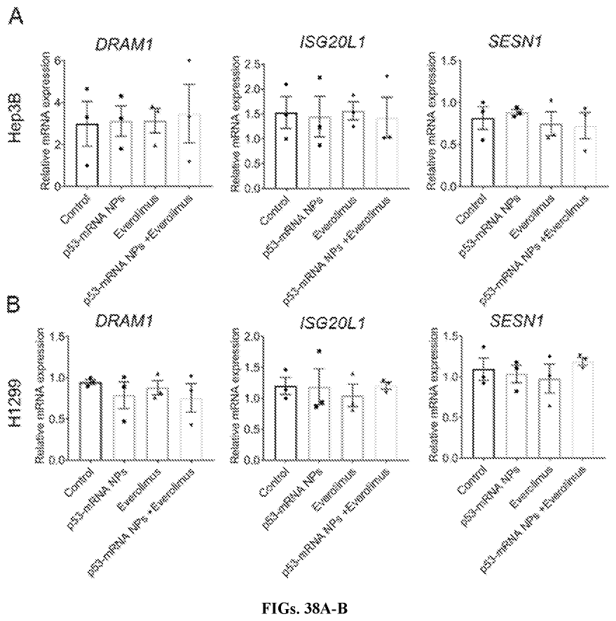

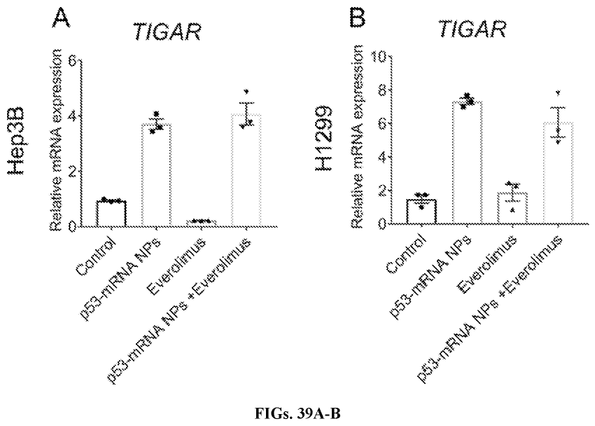

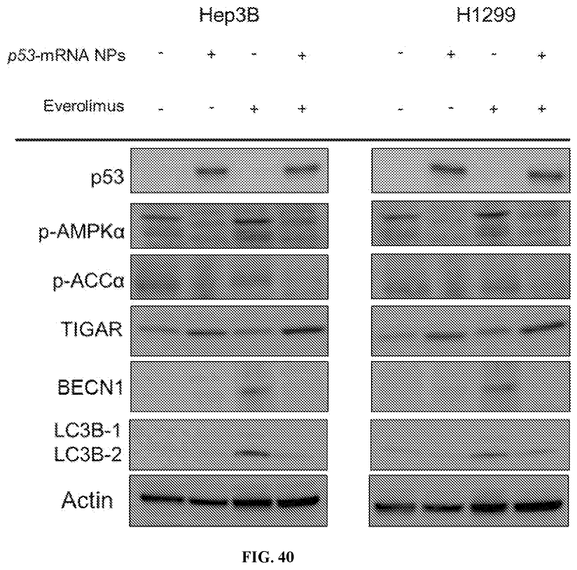

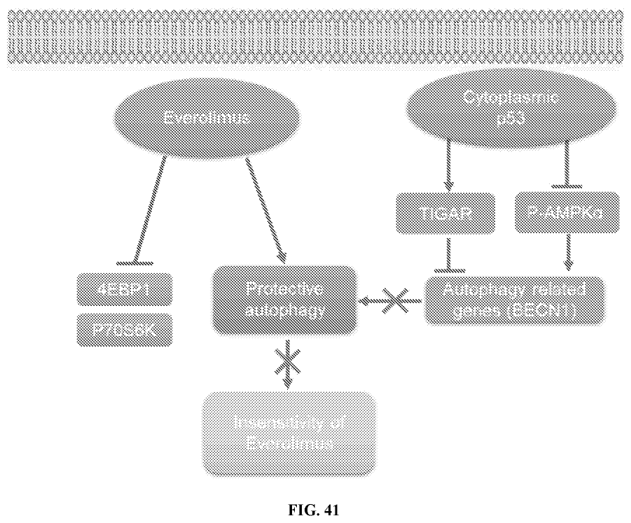

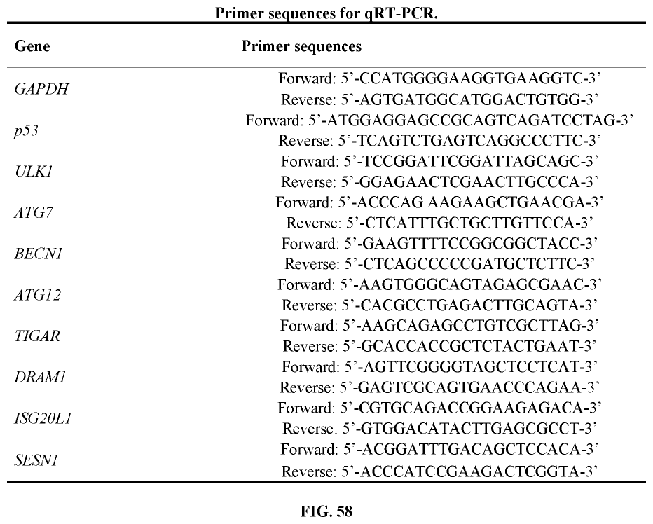

Furthermore, the possible mechanisms of how p53 restoration inhibits the protective autophagy were explored. As shown in the quantitative real time polymerase chain reaction (PCR) results (FIGS. 36 and 58), the intervention of NPs effectively increased the expression of p53 mRNA compared to the groups without NPs treatment in both cell lines. The increased p53 mRNA expression was also accompanied by clear inhibition of ULK1, ATG7, BECN1, and ATG12 mRNA expression (FIG. 37), but showed no obvious effects on the mRNA expression of DRAM1, ISG20L1, and SESN1 (FIG. 38). These results indicate that the autophagy-related genes ULK1, ATG7, BECN1, and ATG12 may be involved in the p53 mRNANP-mediated inhibition of autophagy activation. We also examined two p53 target genes, TIGAR (TP53-induced glycolysis and apoptosis regulator) and AMPKα. TIGAR is a p53-regulated gene that can be rapidly activated in response to cellular stress (53). TIGAR can inhibit autophagy in a transcription-independent manner (54, 55). Consistent with previous studies (54-56), both our PCR and WB results (figs. 39 and 40) demonstrated that the expression of cytoplasmic p53 via p53-mRNANPs activated the expression of TIGAR. The WB data also indicated the suppression of the AMPK signaling pathway (23, 57), which can induce transcription-independent inhibition of autophagy (58). Based on these results, a possible mechanism (FIG. 41) was proposed of how p53 tumor suppressor inhibits the protective autophagy and thus improves the sensitivity of p53-null tumor cells to everolimus.

### Example 4—p53 Restoration Sensitizes p53-Null HCC and NSCLC Xenograft Models to Everolimus

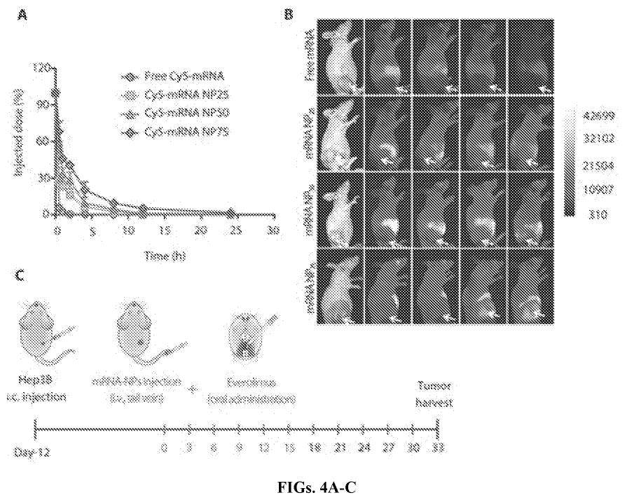

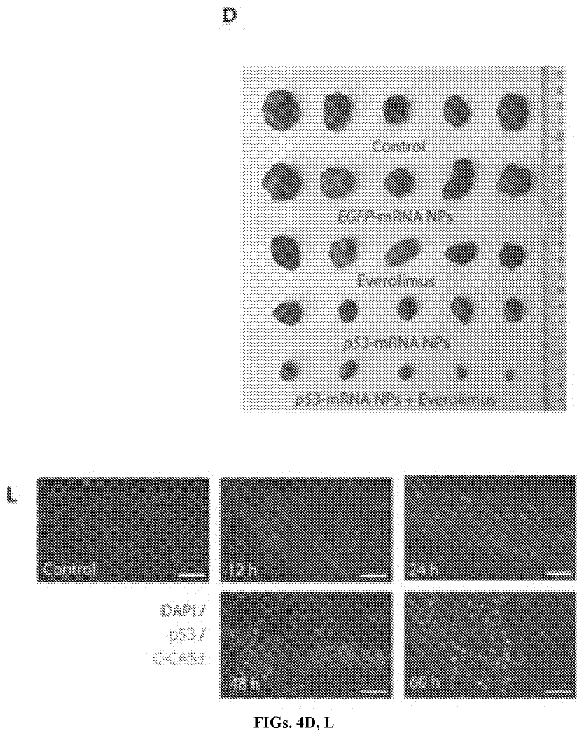

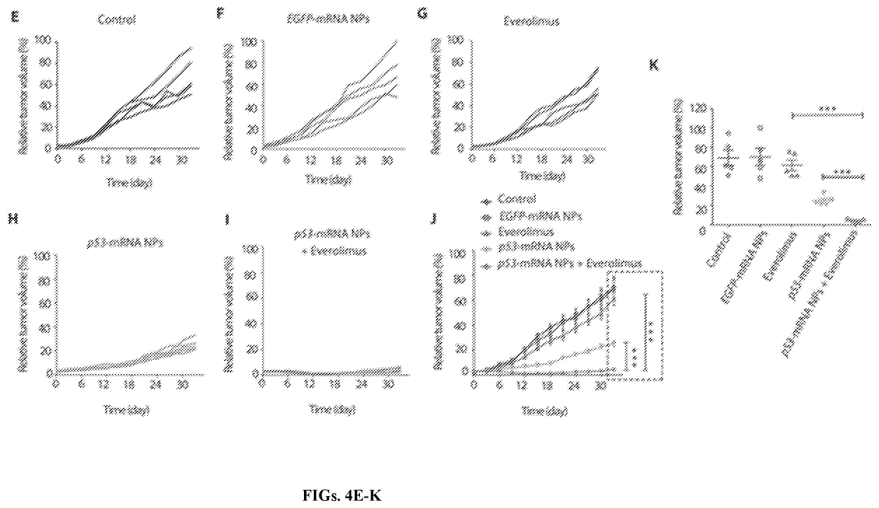

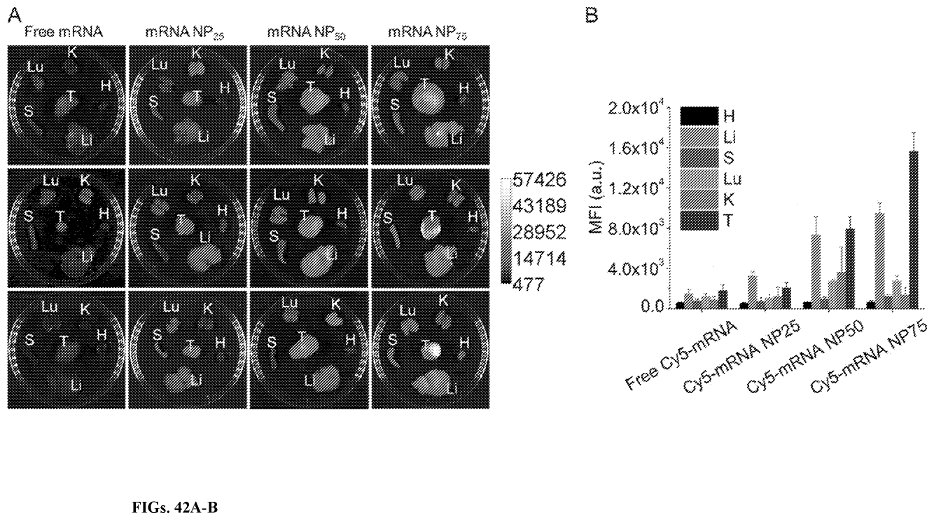

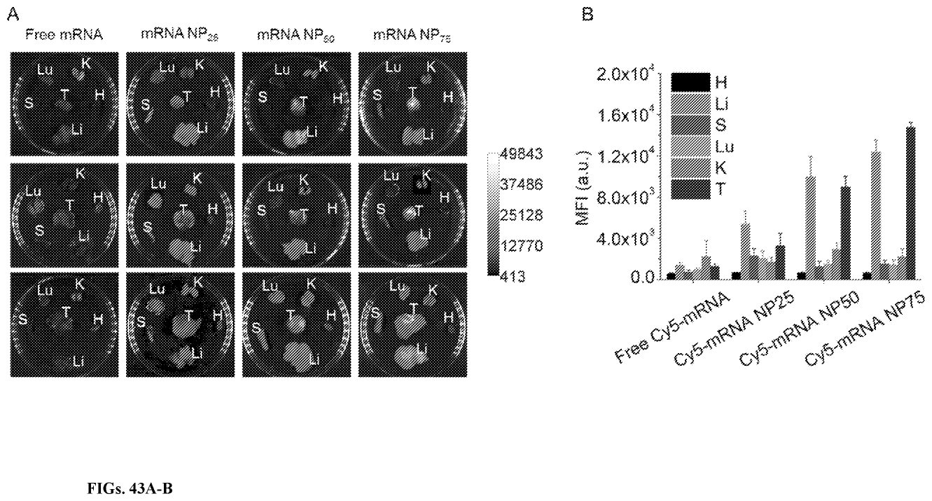

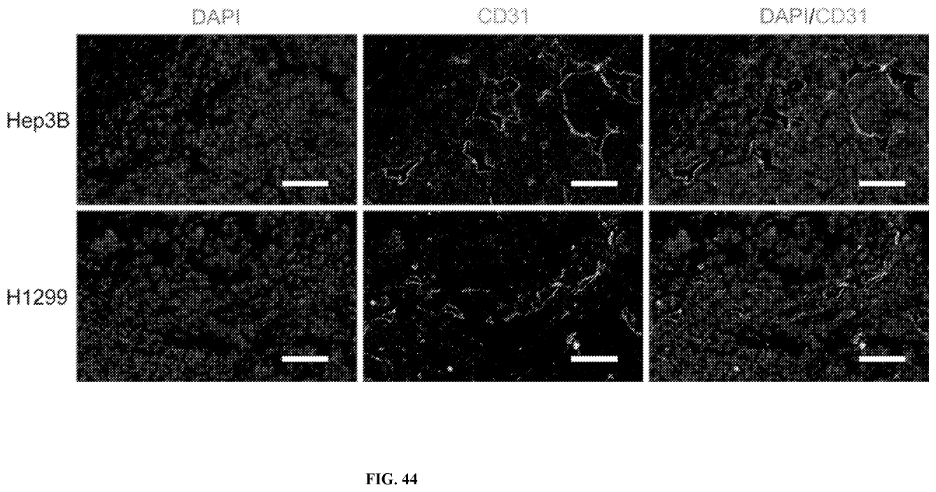

The lipid-PEG layer plays a critical role in controlling the cell uptake, pharmacokinetics (PK), and tumor accumulation of the hybrid lipid-polymer NPs (38, 39). The hybrid mRNA NPs were prepared with three different DSPE-PEG/DMPE-PEG ratios (NP₂₅, NP₅₀, and NP₇₅ shown in rig. 56). PK of the three Cy5-labeled mRNANPs delivered by intravenous (IV) injection into healthy BALB/c mice were evaluated. Naked Cy5-mRNA was used as a control. FIG. 4A shows that naked mRNA was cleared within a few minutes, whereas the hybrid NPs effectively extended the circulation half-life (t_{1/2}) of mRNA (NP₂₅: t_{1/2}<30 min; NP₅₀: t_{1/2}˜30 min; NP₇₅: t_{1/2}˜1 hour). In addition, ˜40% of NP₇₅ were still circulating in blood at 2 hours after administration. We then examined the biodistribution (BioD) and tumor accumulation of these NPs. Athymic nude mice carrying Hep3B xenograft were treated with naked Cy5-mRNA, Cy5-mRNA NP₂₅, Cy5-mRNA NP₅₀, or Cy5-mRNA NP₇₅ by IV injection. As revealed in FIG. 4B and FIG. 42, the fluorescent signal of naked Cy5-mRNA was barely detectable in the tumor at 24 hours after injection. Among the three different NPs, NP₇₅ exhibited the highest tumor accumulation, which may be attributable to its long circulation, and was thus used for all the following in vivo studies. A comparable NP accumulation was also observed in H1299 xenograft tumors (FIG. 43), which may be due to the abundant blood vessels in these two tumor models (FIG. 44).

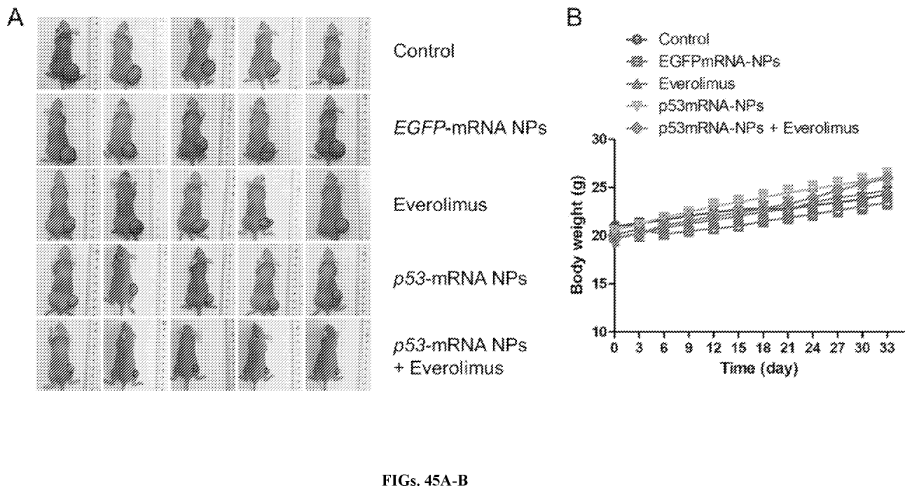

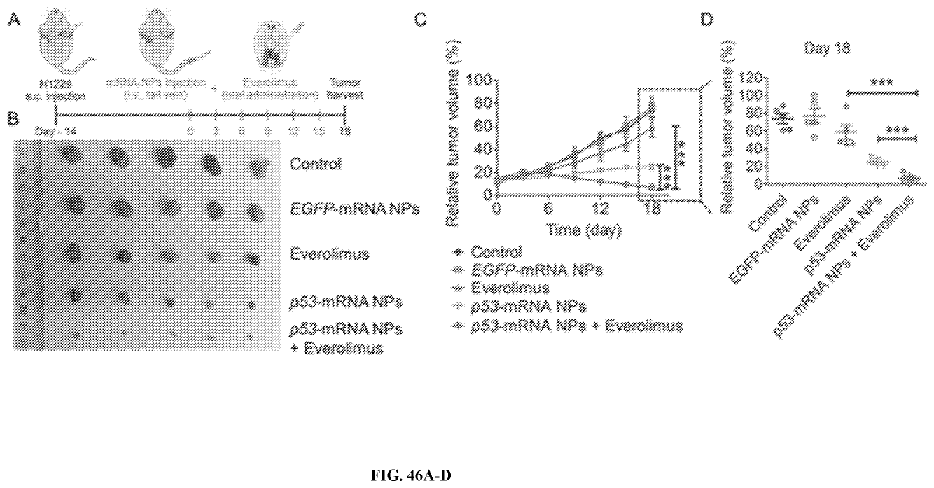

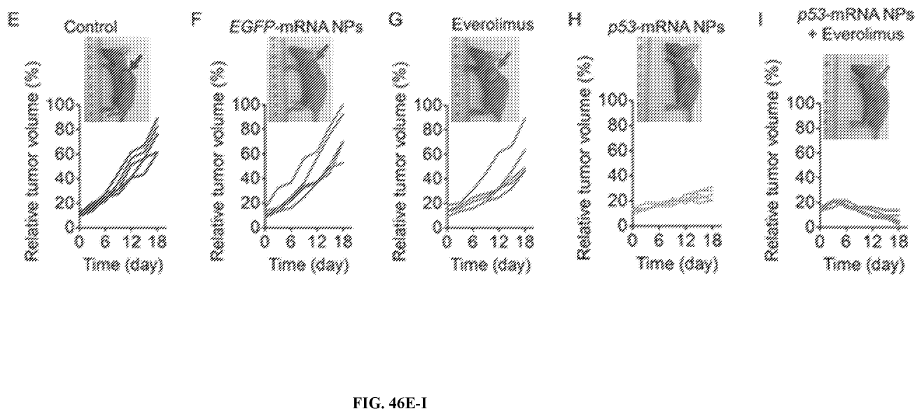

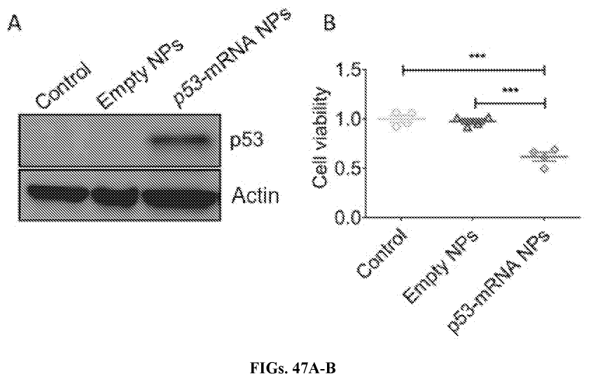

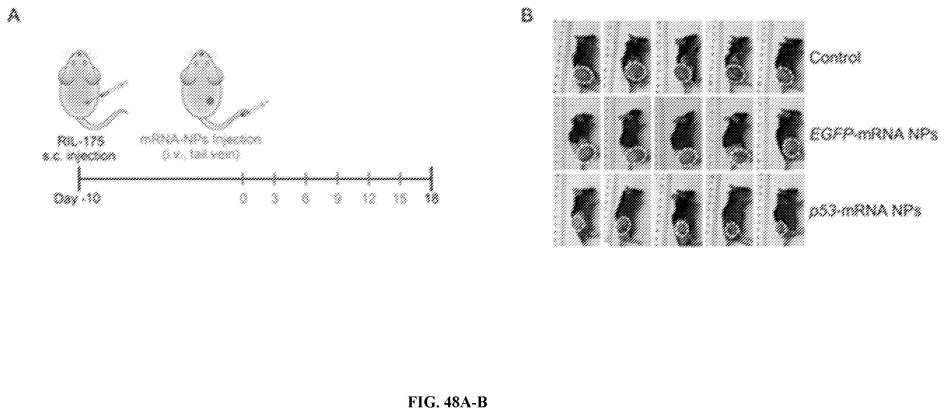

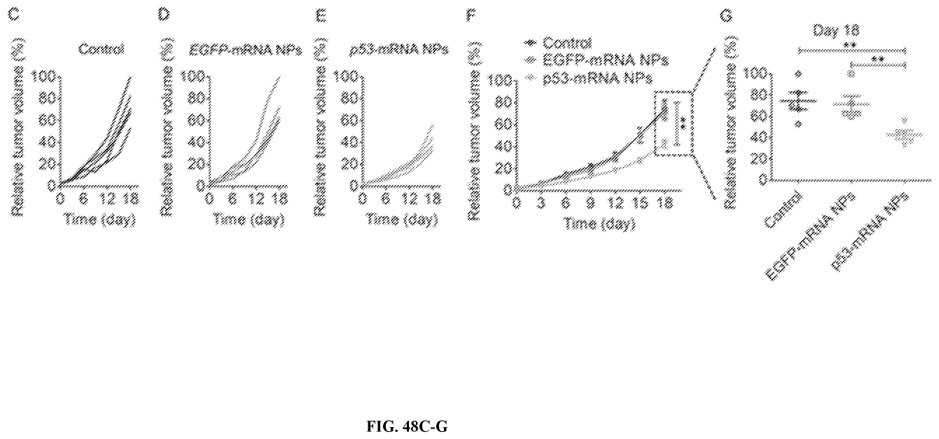

To validate the therapeutic efficacy of the p53-mRNA NPs and their ability to sensitize tumors to everolimus, in vivo studies were performed in immunocompromised athymic nude mice bearing p53-null Hep3B xenografts (FIG. 4C). The p53-mRNANPs were systemically injected via tail vein every three days for six treatments. Meanwhile, everolimus was administered orally right after each IV injection of NPs. PBS and EGFP-mRNANPs were used as controls. Hep3B tumor-bearing mice treated with PBS and EGFP-mRNA NPs showed similarly rapid tumor growth, whereas everolimus alone showed moderate anti-tumor activity (FIG. 4D-K and FIG. 45A). The p53-mRNA NPs demonstrated a potent effect on suppressing the growth of Hep3B tumors. Notably, co-treatment with everolimus and p53-mRNA NPs greatly enhanced the therapeutic efficacy, compared to the treatment with everolimus alone or p53-mRNA NPs at the end point of this study. The CI value was 5.08, indicating a potent synergistic effect of everolimus in combination with p53-mRNA NPs in vivo. No obvious change in body weight was observed in any groups (FIG. 45B). In addition, the combination treatment was highly effective in vivo inp53-null H1299 xenograft tumors (FIG. 46). The CI value was 2.87 for the combination of everolimus with p53-mRNA NPs. The co-treatment even resulted in regression of the H1299 tumors. Moreover, the p53 restoration strategy also worked in the immunocompetent mouse tumor model of p53-null RIL-175, as evidenced by the inhibition of tumor growth after treatment with murine p53-mRNA NPs (figs. 47 and 48).

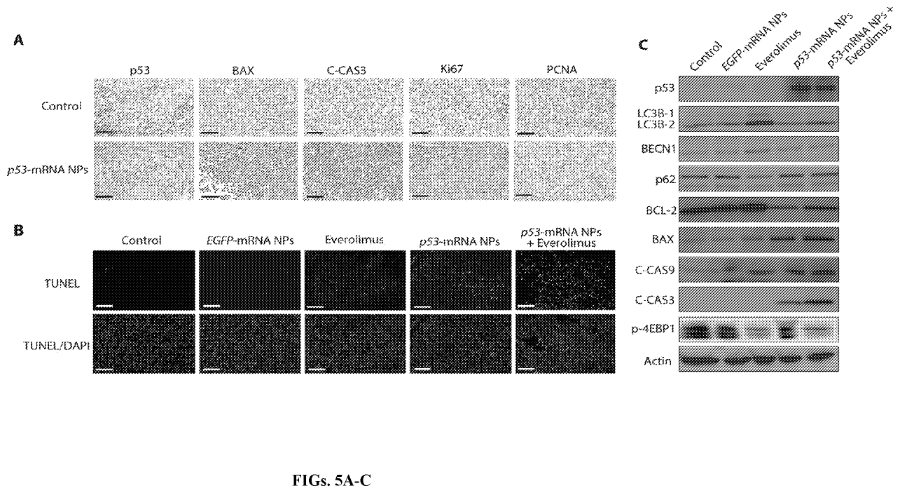

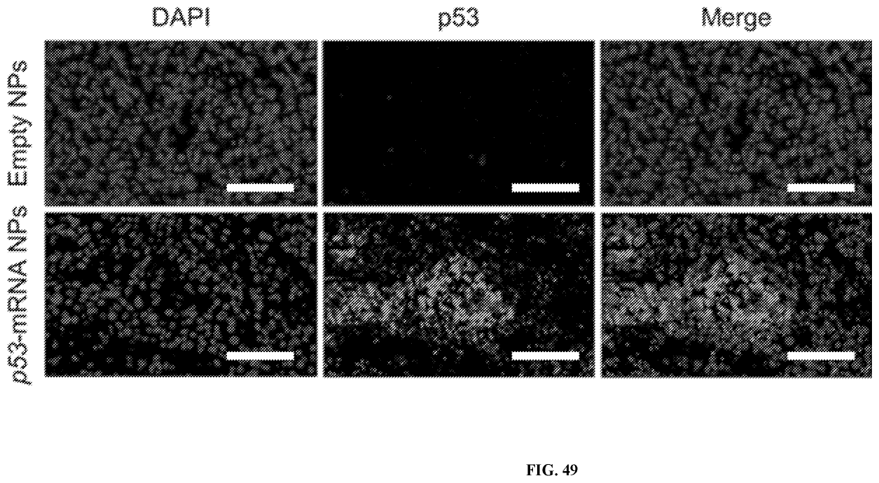

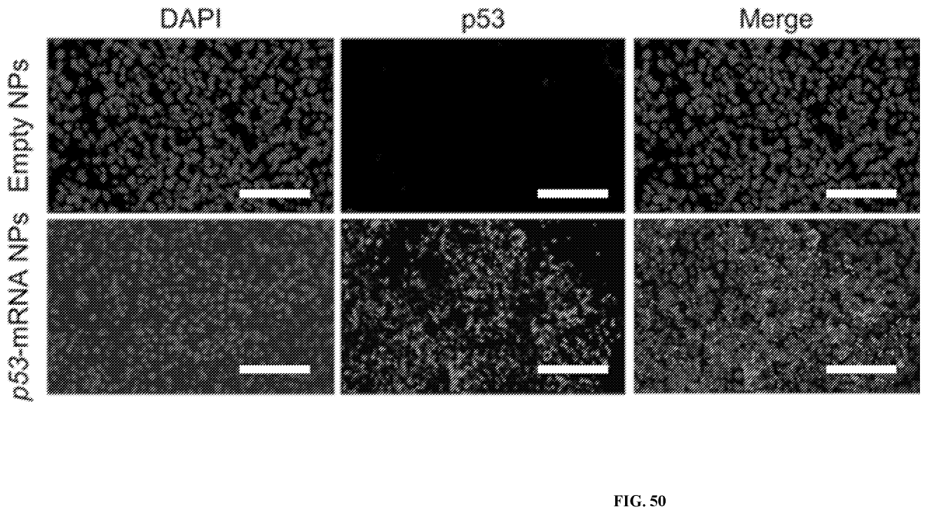

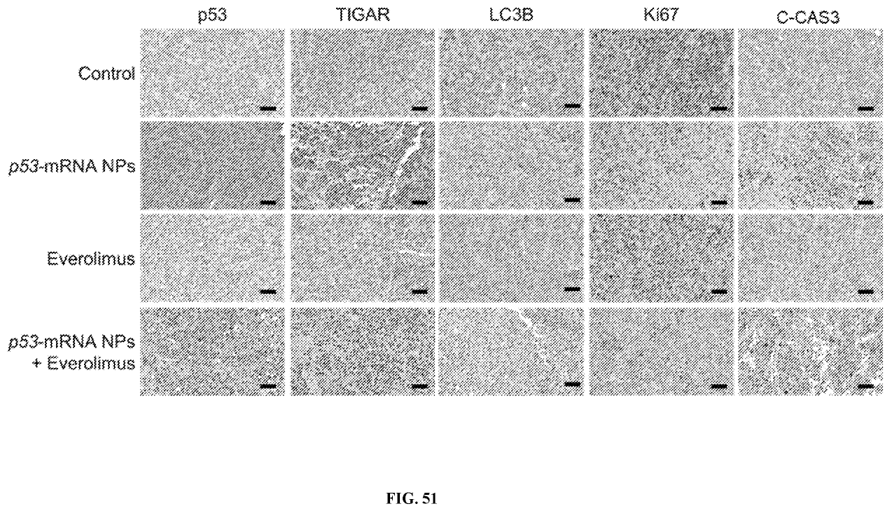

To better understand the in vivo mechanisms underlying this anti-tumor effect, p53 expression inp53-null Hep3B tumor sections obtained at different time points (12, 24, 48, and 60 hours) was tested after three injections of p53-mRNANPs by IF analysis (PBS treatment was used as control). FIG. 4L shows p53 protein expression in tumor sections at all these time points, and the signals were still clear at 60 h after treatment. We also detected upregulated signals of C-CAS3, indicating the apoptosis pathway activated by these p53-mRNANPs. PBS control group did not show any signal of p53 or C-CAS3. Furthermore, immunohistochemistry (IHC) analysis confirmed the high expression of p53 inp53-null Hep3B tumor sections (FIG. 5A), along with the high expression of C-CAS3 after treatment with p53-mRNA NPs. These results indicated the activation of the apoptotic pathway, consistent with the in vitro results. It was also observed that the restored p53 proteins were mainly located in the cytoplasm of Hep3B and H1299 cells in vivo (figs. 49 and 50). Tumor cell proliferation was assessed by Ki67 (proliferation marker) and PCNA (proliferating cell nuclear antigen) expression, both of which were decreased after treatment with p53-mRNA NPs. In addition, TUNEL (terminal deoxynucleotidyl transferase dUTP nick end labeling) assay in tumor sections (FIG. 5B) confirmed that p53-mRNA NP treatment activated the apoptosis pathway. Furthermore, p53 restoration-mediated sensitization to everolimus was examined in vivo. Proteins from Hep3B tumors in different treatment groups were extracted and analyzed by WB. As shown in FIG. 5C, everolimus induced autophagy, as indicated by the expression of LC3B-2 relative to actin (50), as well as the increase in Beclin 1 (BECN1), whereas the co-treatment with p53-mRNA NPs reduced autophagy activation to levels comparable to the control groups. Apoptosis (C-CAS9 and C-CAS3) was enhanced in the “p53-mRNA NPs+everolimus” group. The mTOR and autophagic pathways in p53-null NSCLC xenograft model were also analyzed via IHC studies (FIG. 51). The expression of major proteins (p53, TIGAR, LC3B, Ki67, and C-CAS3) involved in the pathways discussed above was verified in the H1299 tumor sections. Treatment with p53-mRNA NPs resulted in the expressions of p53 and TIGAR and inhibited the LC3B (autophagy marker) expression induced by everolimus. The down-regulation of Ki67 and up-regulation of C-CAS3 indicated activation of the apoptosis pathway.

### Example 5—In Vivo Therapeutic Efficacy in p53-Null Orthotopic HCC Model and Disseminated NSCLC Model

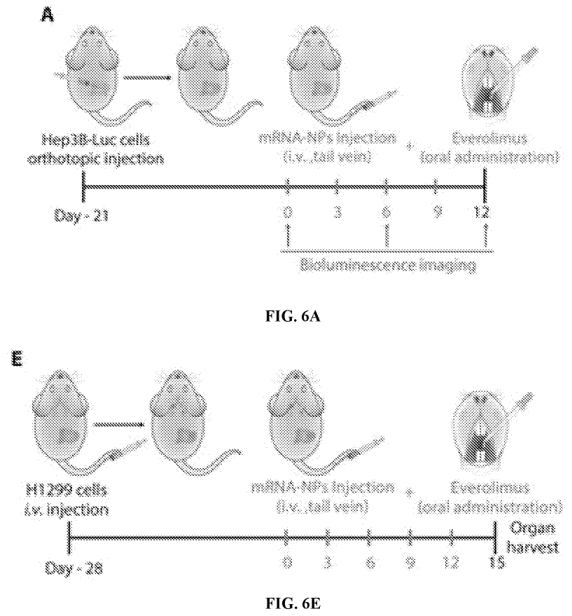

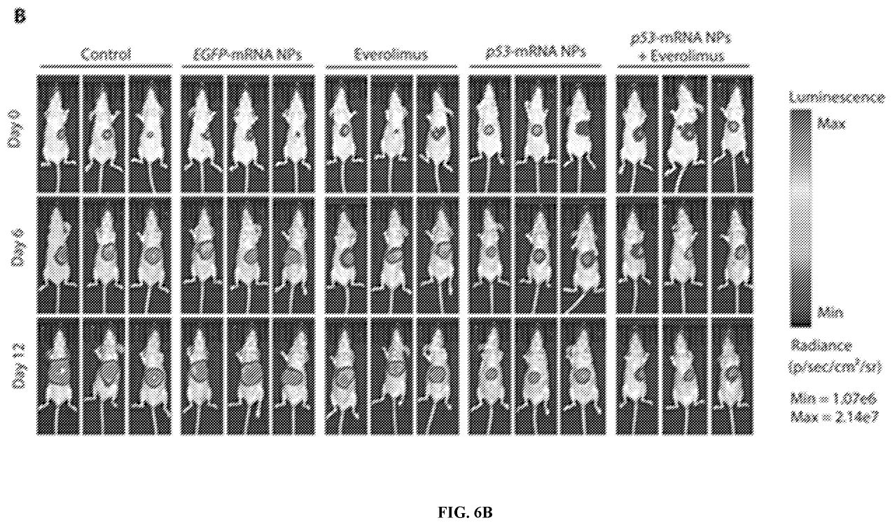

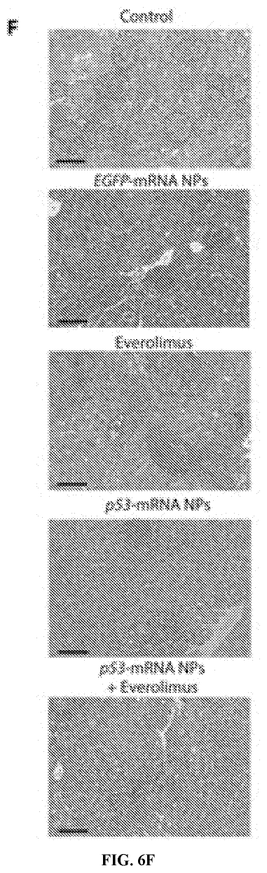

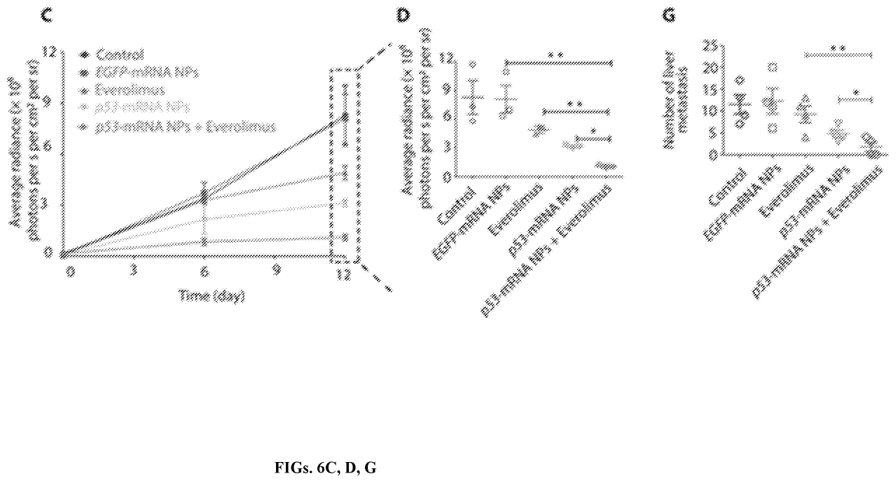

To further evaluate the therapeutic efficacy of p53-mRNA NPs in combination with everolimus, a p53-null orthotopic model of HCC was established by injecting luciferase-expressing Hep3B (Hep3B-Luc) cells into the left lobe of the livers of immunodeficient nude mice. Tumor growth was monitored by detecting the average radiance of the tumor sites through bioluminescence imaging. Twenty-one days later, mice were randomly divided into different groups and treated with PBS, EGFP-mRNANPs, everolimus, p53-mRNANPs, or p53-mRNA NPs+everolimus every three days (FIG. 6A). Everolimus was orally administered, whereas PBS and all NPs were given by IV injection. Bioluminescence imaging was performed on Day 0, Day 6, and Day 12. As shown in FIG. 6B, everolimus somewhat inhibited the growth of orthotopic tumors, as compared to the PBS and EGFP-mRNA NPs groups. p53-mRNANPs effectively reduced the orthotopic tumor burden, and co-treatment with p53-mRNA NPs and everolimus showed the strongest therapeutic effect in the orthotopic model (FIG. 6C).

An experimental liver metastasis was also used as a model to evaluate this combination strategy by IV injection of the H1299 NSCLC cells into immunodeficient mice via the tail vein. Four weeks later, all the mice were randomly assigned to different groups and treated with PBS, EGFP-mRNA NPs, everolimus, p53-mRNA NPs, or p53-mRNA NPs+everolimus every three days (FIG. 6D). After five rounds of treatment, all mice were sacrificed and their livers were collected to detect metastases (FIG. 6E, F, and FIG. 55). Numerous metastatic nodules were detected in the livers from the PBS and EGFP-mRNA NPs groups, and everolimus showed moderate effects. In comparison, p53-mRNA NPs effectively reduced the number of metastatic nodules, whereas co-treatment with p53-mRNA NPs and everolimus showed the most profound therapeutic effect.

### Example 6—In Vivo Safety of p53-mRNA NPs and their Combination with Everolimus

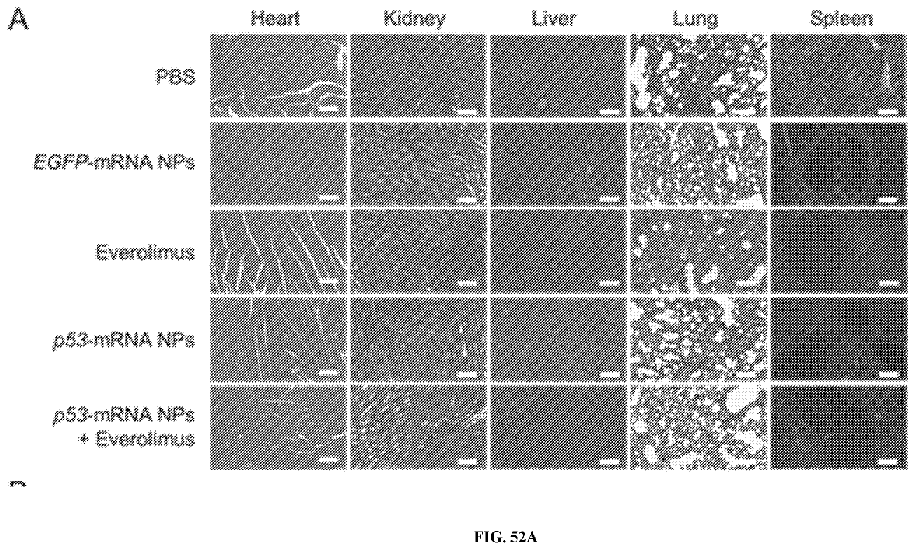

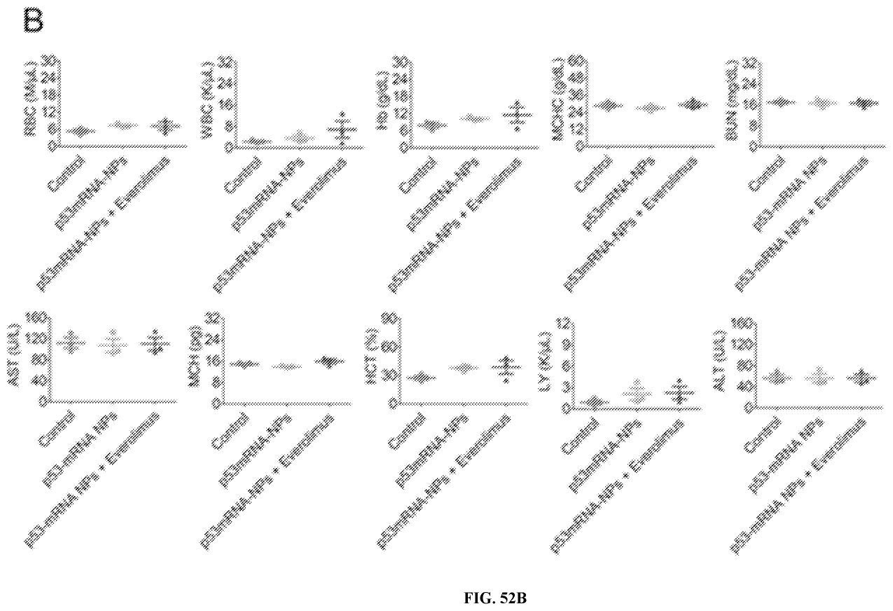

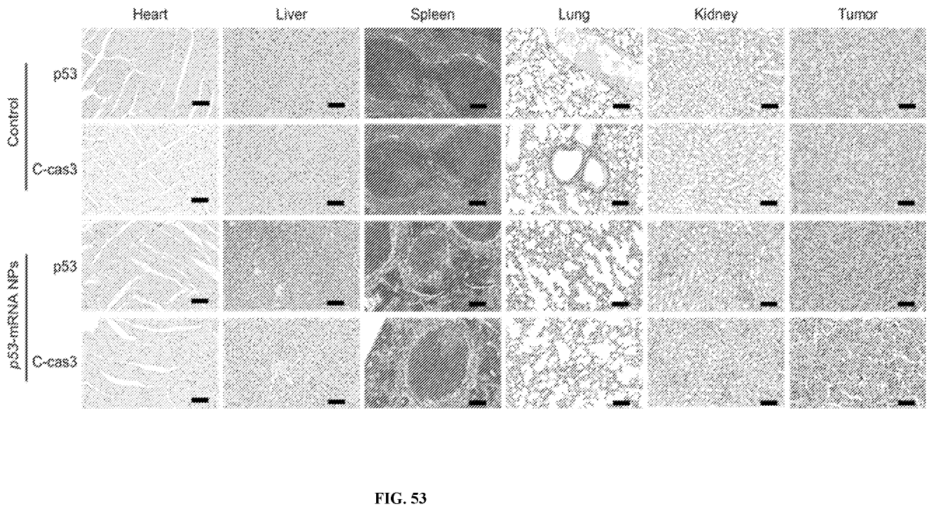

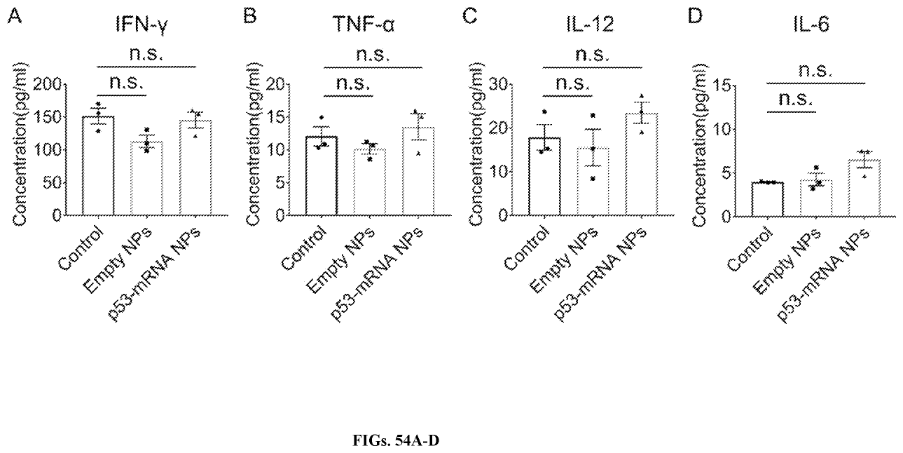

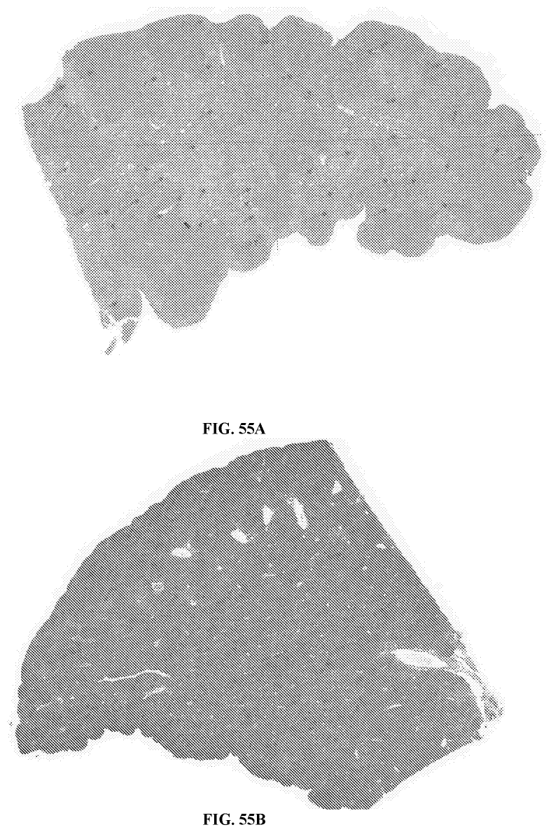

To evaluate the in vivo safety of p53-mRNANPs and their combination with everolimus, various organs (heart, kidneys, liver, lungs, and spleen) were harvested at the end point (day 33) of the Hep3B xenograft study, followed by section and H&E staining (FIG. 52A). No obvious histological differences were detected in the sections of organs from all the treatment groups, indicating no notable toxicity. Serum biochemistry analysis and whole blood panel tests were also performed. A series of parameters were tested (FIG. 52B), including alanine aminotransferase (ALT), aspartate aminotransferase (AST), blood urea nitrogen (BUN), red blood cells (RBC), white blood cells (WBC), hemoglobin (Hb), mean corpuscular hemoglobin concentration (MCHC), mean corpuscular hemoglobin (MCH), hematocrit (HCT), and lymphocyte count (LY). These parameters did not show significant differences between the groups treated with PBS, p53-mRNA NPs, and p53-mRNA NPs+everolimus. Moreover, IHC analysis was performed for the expressions of p53 and C-CAS3 in major organs (heart, liver, spleen, lungs, and kidneys) and tumors. As can be seen in FIG. 53, p53 was mainly expressed in the tumor and liver, which is consistent with the biodistribution results (with the NP delivery platform, mRNA had higher accumulation in the tumor and liver). The restoration of p53 in p53-null HCC tumors resulted in effective expression of C-CAS3, consistent with in vitro studies. In addition, no obvious expression of C-CAS3 was observed in normal tissues including the liver, which is consistent with H&E staining results. Moreover, blood serum concentrations of immuno-toxicity markers such as interferon gamma (IFN-γ), tumor necrosis alpha (TNF-α), interlukin-12 (IL-12), and interlukin-6 (IL-6) were in the normal range at 24 h after treatment with either empty NPs or p53-mRNA NPs (FIG. 54). These results indicated that no observable innate immune responses were caused by the mRNA NPs at the tested time point.

### Discussion of Examples 1-6

The p53 gene is a critical tumor suppressor gene involved in the majority of cancers (59, 60). The clinical data from TCGA show that both HCC and NSCLC patients with high expression of p53 have much longer overall survival and/or progression-free survival than those with low p53 expression (61, 62). With its diverse functions (such as regulation of cell cycle checkpoints, apoptosis, senescence, and DNA repair), p53 restoration has long been considered an attractive anti-cancer strategy (63-65). Various methods have been developed to reactivate p53 functions, which can be summarized in the two categories of small molecular compounds (25-27) and DNA therapeutics (29, 30). Small molecular inhibitors, such as RITA (Reactivation of p53 and Induction of Tumor cell Apoptosis), Nutlin, and MI-319, have shown high binding potency and selectivity for MDM2 in the treatment of HCC and other cancers (66-68). Other small molecules like CP-31398 have also been developed to target mutant p53 and reactivate its normal functions (69, 70). Encouraging clinical outcomes are being continually generated with compounds such as RG7112, MI-773, and APR-246 in different cancers. For example, the Phase I trial of RG7112 (an MDM2 antagonist) has demonstrated clinical responses in hematologic malignancies (71). MI-773 (SAR405838; an HDM2 antagonist) was shown to be safe with preliminary anti-tumour activity in locally advanced or metastatic solid tumours (72). In addition, combination treatment with APR-246 and azacitidine (AZA) resulted in responses in all patients with TP53-mutant myelodysplastic syndromes and acute myeloid leukemia in a Phase Ib/II study (73). Despite these efforts and the progress in clinical trials (32), this method is likely to be ineffective when the suppressor gene has been deleted. For DNA therapeutics, several candidates using adenoviral vectors are in clinical trials, with Gendicine approved in China in 2003 (74). Advexin, another Adp53 vector, however, failed in the Phase III trials (75). Considering the low transduction rate of p53 gene via Adp53 (76), some tumor-specific, replication-competent CRAdp53 vectors (AdDelta24-p53, SG600-p53, ONYX 015, OBP-702, and H101) have been developed to induce higher p53 expression and anti-tumor effect. SGT-53, a cationic liposome encapsulating p53 plasmid, is also in clinical trials for solid tumors (31). Although Gendicine and H101 have been approved for head and neck cancers in China (76), they are not widely used, presumably due to the limitations of intratumoral injection. Furthermore, gene therapy for systemic cancer treatment still has several potential risks, including i) host immune responses and pre-existing anti-viral immunity resulting in the neutralization of efficacy, modification of PK and pharmacodynamics, and allergic responses; and ii) potential genotoxicity owing to integration in the host genome (33).

The use of synthetic mRNA has recently attracted considerable attention owing to its distinctive features. For example, it does not require nuclear entry for transfection activity and has a negligible chance of integrating into the host genome, thus avoiding potentially detrimental genotoxicity (34, 35). Chemical modification of mRNA molecules has also enhanced their stability and decreased activation of innate immune responses (37). Whereas the use of mRNA to restore tumor suppressors seems straightforward and highly promising, effective systemic delivery of mRNA to tumors remains a major challenge. Nanotechnology has shown promise to improve cytosolic delivery of various RNA therapeutics into tumor cells (77, 78), and different NP systems have been developed for systemic mRNA delivery (79-81), particularly to the liver for genetic and infectious diseases (82-88). However, little efforts have been reported on systemic delivery of mRNA for restoration of tumor suppressors.

A lipid-polymer hybrid mRNA NP platform composed of poly(lactic-co-glycolic acid) (PLGA) was developed and successfully applied it for in vivo restoration of tumor suppressor PTEN in prostate cancer (40). Considering the fact that the concentration of reductive agent GSH in tumor cells could be approximately 100- to 1000-fold higher than that in the extracellular fluids (89), redox-responsive NP platforms have emerged for effective intracellular delivery (41-47), which may be particularly beneficial for biomacromolecules that need to be released into the cytoplasm for therapeutic effects.

The methods within the present claims include, among other things, a redox-responsive polymer PDSA in the hybrid NP platform, which showed a fast mRNA release in the presence of reductive agent DTT and resulted in excellent mRNA transfection. In addition, the reduced EGFP protein expression after the quenching of intracellular GSH by Nem also suggested that redox-responsive NPs might be more potent for mRNA delivery than non-responsive NPs. In addition to the polymer core, the surface lipid-PEG layer also plays an important role in controlling the performance (cellular uptake and PK) of the hybrid NPs for delivery of RNA therapeutics by serum albumin-mediated de-PEGylation (38, 39). For instance, DSPE-PEG contributes to a long circulation life and high tumor circulation due to its slow dissociation from NPs, whereas DMPE-PEG contributes to a high cellular uptake and excellent in vitro performance of the hybrid NPs due to its quick de-PEGylation kinetics. The methods within the present claims use, e.g., two lipid-PEG molecules by changing the DSPE-PEG/DMPE-PEG ratio for different in vitro or in vivo applications. To maximize the tumor accumulation, the lipid-PEG layer of NPs needs to be relatively stable (with a slow de-PEGylation kinetic profile) to enable a relatively long circulation time. Therefore, a high ratio of DSPE-PEG (75%, w/w) to the total lipid-PEGs on the surface layer was designed for systemic delivery of mRNA. Compared with the PLGA-based NPs coated with a layer of single lipid-PEG (40), the PDSA-based NPs coated with a layer of hybrid lipid-PEGs are more adjustable for on-demand applications.

Previous studies (11-13) have shown that activation of autophagy by mTOR inhibitors including everolimus may be an undesired effect because it acts as a resistance mechanism that limits drug efficacy. The incorporation of autophagy inhibitors could prevent resistance to mTOR inhibitors and enhance their therapeutic efficacy. For example, a dual mTORC1 and mTORC2 inhibitor, OSI-027, was reported to induce protective autophagy, whereas disruption of this pathway with chloroquine (autophagy inhibitor) contributed to apoptotic cell death (90). Both selective knockdown of autophagy genes (ATG3, ATG5, and ATG7) and pre-treatment with hydroxychloroquine (autophagy inhibitor) also contributed to activating the mitochondrial apoptotic pathway and improving everolimus activity, sensitizing mantle cell lymphoma to everolimus (10). Interestingly, p53 plays a dual role in control of autophagy. (i) nuclear p53 can induce autophagy through transcriptional effects, whereas (ii) cytoplasmic p53 can act as a master repressor of autophagy (57, 91). In this work, we observed that the p53 proteins restored by mRNA NPs are mainly located in the cytoplasm of both Hep3B and H1299 cells in vitro and in vivo. In addition, we observed that everolimus-induced autophagy activation was effectively inhibited by mRNA NP-based restoration of p53, further demonstrating the expression of p53 proteins mainly in the cytoplasm.

In summary, the experiments of the present disclosure demonstrate that p53 restoration by synthetic mRNA NPs can inhibit autophagy, thus providing a strategy for sensitizing p53-null tumor cells to everolimus, and simultaneously activate apoptosis and cell cycle arrest. The redox-responsive p53-mRNA NPs enhanced the therapeutic responses to everolimus in p53-null HCC and NSCLC in vitro and in vivo. A synergistic anti-tumor effect was also observed in multiple animal models of both HCC and NSCLC with the combinatorial treatment, which might be explained by (i) the mild therapeutic effect of everolimus, (ii) cytoplasmic p53-mediated inhibition of autophagy and sensitization to the mTOR inhibitor, and (iii) the simultaneous activation of apoptosis by p53 restoration. The synthetic mRNA NP-based p53 restoration strategy might therefore revive this FDA-approved mTOR inhibitor for clinical translation in p53-deficient HCC and NSCLC patients.

### Example 7—Cell Viability Evaluation of Human p53 mRNA NPs with Cisplatin or Metformin

Experimental Methods. Three lung cancer cell lines, including A549 (p53 wild type), H1299 (p53 deficiency), and H1975 (p53 mutation), were cultured with RPMI 1640 media and plated in 96-well plates with the cell density of 6000 cells/mL. After 24 h incubation, the cells were treated with cisplatin, human p53 mRNA NPs, control NPs (without p53), or the combination of p53 mRNA NPs with cisplatin for 24 h and then 100 μL fresh media were added to the treated cells for another 24 h incubation. Then, the cell viabilities of these cells were measured by Alamar blue assay. The concentration of p53 mRNA was 1 μg/mL, while the concentrations of cisplatin were set at 10 or 20 μg/mL (for A549 cells), 5 or 10 μg/mL (for H1299 cells), and 10 or 15 μg/mL (for H1975 cells). In cisplatin treatment groups, the lower concentration was denoted as “Cis-1” and the higher concentration was denoted as “Cis-2”. The cells without receiving any treatments were labeled as the “Control”.

For the cell viability evaluation of human p53 mRNA and metformin, the procedures were same as those described above, except for the metformin concentrations. The concentrations of metformin were set at 4 or 6 mg/mL (for A549 cells), 6 or 8 mg/mL (for H1299 cells), and 3 or 4 mg/mL (for H1975 cells).

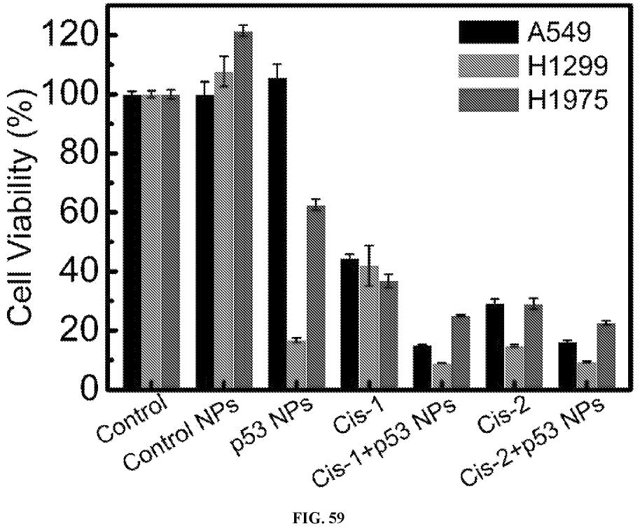

Experimental Results. As shown in FIG. 59, the control NPs induced no toxicity to the three kinds of cells, indicating the good biocompatibility of the mRNA NPs. After the treatment of p53 mRNA NPs (denoted as “p53 NPs” in the figure), negligible cell death was observed with A549 cells, while ˜40% and >80% cell deaths for H1975 and H1299 cells, respectively, were noticed. In the “Cis-1/2+p53 NPs” groups, A549 cells were efficiently killed by the combination of cisplatin and p53 mRNA NPs with higher mortality (80%-90%) than cisplatin-treated groups (60%-70%) at both concentrations of cisplatin. For H1299 and H1975, the cell mortality induced by “Cis-1/2+p53 NPs” was also higher than that caused by cisplatin or p53 mRNA NPs. In conclusion, the combination of cisplatin and p53 mRNA NPs may lead to a synergistic anti-tumor effect in A549 cells, while more p53 concentrations will be tested for H1299 and H1975. The varied p53 status of different lung cancer cell lines might also be responsible for the differences we observed, and p53 mutation is variable even among lung cancer patients. Besides, the possible mechanisms about the synergistic effect of cisplatin and p53 mRNA NPs might be attributable to p53-mediated enhancement of cell apoptosis and caspase-3 activity in cisplatin-treated cells. It has been reported that apoptosis induced by cisplatin would be markedly reduced in the tumor cells that have no p53 mutation. On the other hand, the effects on p53 expression induced by cisplatin treatment may also be a vital factor to determine the anticancer outcome of cisplatin in combination with p53 mRNANPs.

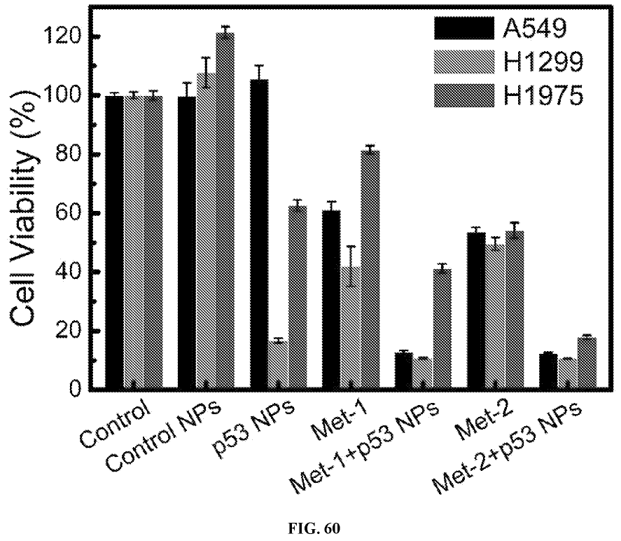

For the combination of metformin with p53 mRNA NPs (FIG. 60), about 90% of A549 cells were dead after the co-treatments at both concentrations of metformin, while less than 50% of A549 cells were killed by the cisplatin alone and there is no cytotoxicity by p53 mRNANPs. This result indicates the much higher and synergistic cytotoxicity (˜90%) induced by the combination of metformin and p53 mRNA NPs. For H1299 cells, due to the very high toxicity by p53 mRNA NPs, the combination group showed negligible advantages on cell killing. Lower p53 concentrations will need to be tested for the combination in H1299 cells. For H1975 cells, the mortality in “Met-2+p53” group (˜90%) was much higher than that in “Met-2” or “p53” groups (˜50% and 40%, respectively), indicating that a highly improved therapeutic efficiency could be achieved by the combinatorial treatment. Consequently, the combination of p53 and metformin showed higher anti-tumor effects in lung cancer cells. The corresponding mechanism of the combination of metformin and p53 mRNA NPs might be attributable to the more activation of AMPK phosphorylation followed by more inhibition of mTOR phosphorylation and augmentation of cleaved caspase 3 compared with metformin or p53 mRNA NPs alone. This might be involved with the blockage action of metformin to alternative cell survival pathways, such as the mevalonate, metabolic, autophagy, proteasome, and PDGFR pathways.

### REFERENCES

1. M. Laplante, David M. Sabatini, mTOR Signaling in Growth Control and Disease. Cell 149, 274-293 (2012).
2. J. Dancey, mTOR signaling and drug development in cancer. Nat. Rev. Clin. Oncol. 7, (2010).
3. A. Yoshizawa et al., Overexpression of Phospho-eIF4E Is Associated with Survival through AKT Pathway in Non-Small Cell Lung Cancer. Clin. Cancer Res. 16, 240-248 (2010).
4. A. Villanueva et al., Pivotal Role of mTOR Signaling in Hepatocellular Carcinoma. Gastroenterology 135, 1972-1983.e1911 (2008).
5. A. X. Zhu et al., Effect of everolimus on survival in advanced hepatocellular carcinoma after failure of sorafenib: the EVOLVE-1 randomized clinical trial. JAMA 312, 57-(2014).
6. B. Besse et al., Phase II study of everolimus-erlotinib in previously treated patients with advanced non-small-cell lung cancer. Ann. Oncol. 25, 409-415 (2014).
7. A. Tarhini et al., Phase II study of everolimus (RAD001) in previously treated small cell lung cancer. Clin. Cancer Res. 16, 5900-5907 (2010).
8. A. Ohtsu et al., Everolimus for previously treated advanced gastric cancer: results of the randomized, double-blind, phase III GRANITE-1 study. J. Clin. Oncol. 31, 3935-3943 (2013).
9. T. O'Reilly, P. M. J. McSheehy, Biomarker Development for the Clinical Activity of the mTOR Inhibitor Everolimus (RAD001): Processes, Limitations, and Further Proposals. Transl. Oncol. 3, 65-79 (2010).
10. J. S. Carew, K. R. Kelly, S. T. Nawrocki, Mechanisms of mTOR inhibitor resistance in cancer therapy. Target. oncol. 6, 17-27 (2011).
11. L. Rosich et al., Counteracting autophagy overcomes resistance to everolimus in mantle cell lymphoma. Clin. Cancer Res. 18, 5278-5289 (2012).
12. Z. Chen et al., NPRL2 enhances autophagy and the resistance to Everolimus in castration-resistant prostate cancer. Prostate. 79, 44-53 (2019).
13. S. C. Lee et al., Everolimus Plus Ku0063794 Regimen Promotes Anticancer Effects against Hepatocellular Carcinoma Cells through the Paradoxical Inhibition of Autophagy. Cancer Res. Treat. 50, 1023-1038 (2018).
14. M. Marinov et al., AKT/mTOR pathway activation and BCL-2 family proteins modulate the sensitivity of human small cell lung cancer cells to RAD001. Clin. Cancer Res. 15, 1277-1287 (2009).
15. G. Lessene, P. E. Czabotar, P. M. Colman, BCL-2 family antagonists for cancer therapy. Nat. Rev. Drug Discov. 7, 989-1000 (2008).
16. K. U. Wagner et al., Conditional deletion of the Bcl-x gene from erythroid cells results in hemolytic anemia and profound splenomegaly. Development 127, 4949-4958 (2000).
17. T. Kimura, Y Takabatake, A. Takahashi, Y Isaka, Chloroquine in Cancer Therapy: A Double-Edged Sword of Autophagy. Cancer Res. 73, 3 (2013).
18. E. Cerami et al., The cBio Cancer Genomics Portal: An Open Platform for Exploring Multidimensional Cancer Genomics Data. Cancer Discov. 2, 401 (2012).
19. W. Xue et al., Senescence and tumour clearance is triggered by p53 restoration in murine liver carcinomas. Nature 445, 656 (2007).
20. K. D. Sullivan, M. D. Galbraith, Z. Andrysik, J. M. Espinosa, Mechanisms of transcriptional regulation by p53. Cell Death Differ. 25, 133-143 (2018).
21. T. Li et al., Tumor suppression in the absence of p53-mediated cell-cycle arrest, apoptosis, and senescence. Cell 149, 1269-1283 (2012).
22. C. C. Ho et al., Stalled replication induces p53 accumulation through distinct mechanisms from DNA damage checkpoint pathways. Cancer Res. 66, 2233-2241 (2006).
23. E. Tasdemir et al., Regulation of autophagy by cytoplasmic p53. Nat. Cell. Biol. 10, 676-687 (2008).
24. X. Sui et al., Autophagy and chemotherapy resistance: a promising therapeutic target for cancer treatment. Cell Death Dis. 4, e838 (2013).
25. V. J. N. Bykov et al., Reactivation of mutant p53 and induction of apoptosis in human tumor cells by maleimide analogs. J. Biol. Chem. 292, 19607-19607 (2017).
26. Patricia A. Muller, Karen H. Vousden, Mutant p53 in Cancer: New Functions and Therapeutic Opportunities. Cancer Cell 25, 304-317 (2014).
27. V. J. N. Bykov et al., Restoration of the tumor suppressor function to mutant p53 by a low-molecular-weight compound. Nat. Med. 8, 282 (2002).
28. L. T. Vassilev et al., In vivo activation of the p53 pathway by small-molecule antagonists of MDM2. Science 303, 844-848 (2004).
29. V. W. van Beusechem et al., Conditionally Replicative Adenovirus Expressing p53 Exhibits Enhanced Oncolytic Potency. Cancer Res. 62, 6165 (2002).
30. Y S. Guan et al., p53 gene (Gendicine) and embolisation overcame recurrent hepatocellular carcinoma. Gut 54, 1318-1319 (2005).
31. K. F. Pirollo et al., Safety and Efficacy in Advanced Solid Tumors of a Targeted Nanocomplex Carrying the p53 Gene Used in Combination with Docetaxel: A Phase Ib Study. Mol. Ther. 24, 1697-1706 (2016).
32. C. F. Cheok, C. S. Verma, J. Baselga, D. P. Lane, Translating p53 into the clinic. Nat.

Rev. Clin. Oncol. 8, 25 (2010).
33. R. Kapoor, T. Klueter, J. M. Wilson, Challenges in the gene therapy commercial ecosystem. Nat. Biotechnol. 35, 813 (2017).
34. V. F. Van Tendeloo, P. Ponsaerts, Z. N. Berneman, mRNA-based gene transfer as a tool for gene and cell therapy. CURR OPIN MOL THER. Curr. Opin. Mol. Ther. 9, 423-431 (2007).
35. J. B. Ulmer, P. W. Mason, A. Geall, C. W. Mandl, RNA-based vaccines. Vaccine 30, 4414-4418 (2012).
36. S. M. Azimi et al., Combinatorial programming of human neuronal progenitors using magnetically-guided stoichiometric mRNA delivery. eLife 7, e31922 (2018).
37. M. S. Kormann et al., Expression of therapeutic proteins after delivery of chemically modified mRNAin mice. Nat. Biotechnol. 29, 154-157 (2011).
38. X. Zhu et al., Long-circulating siRNA nanoparticles for validating Prohibitin1-targeted non-small cell lung cancer treatment. Proc. Natl. Acad. Sci. USA. 112, 7779-7784 (2015).
39. X. Zhu et al., Surface De-PEGylation Controls Nanoparticle-Mediated siRNA Delivery In Vitro and In Vivo. Theranostics 7, 1990-2002 (2017).
40. M. A. Islam et al., Restoration of tumour-growth suppression in vivo via systemic nanoparticle-mediated delivery of PTEN mRNA. Nat. Biomed. Eng. 2, 850-864 (2018).
41. J. Wu et al., Hydrophobic Cysteine Poly(disulfide)-based Redox-Hypersensitive Nanoparticle Platform for Cancer Theranostics. Angew. Chem. Int. Ed. 54, 9218-9223 (2015).
42. X. Ling et al., Glutathione-Scavenging Poly(disulfide amide) Nanoparticles for the Effective Delivery of Pt(IV) Prodrugs and Reversal of Cisplatin Resistance. Nano Lett. 18, 4618-4625 (2018).
43. X. Xu et al., Redox-Responsive Nanoparticle-Mediated Systemic RNAi for Effective Cancer Therapy. Small 14, e1802565 (2018).
44. Y Lu, A. A. Aimetti, R. Langer, Z. Gu, Bioresponsive materials. Nat. Rev. Mater. 2, (2016).
45. M. Zhao et al., Redox-responsive nanocapsules for intracellular protein delivery. Biomaterials 32, 5223-5230 (2011).
46. F. Meng, W. E. Hennink, Z. Zhong, Reduction-sensitive polymers and bioconjugates for biomedical applications. Biomaterials 30, 2180-2198 (2009).
47. R. Cheng et al., Glutathione-responsive nano-vehicles as a promising platform for targeted intracellular drug and gene delivery. J. Control. Release 152, 2-12 (2011).
48. M. Mancini et al., Mitochondrial Proliferation and Paradoxical Membrane Depolarization during Terminal Differentiation and Apoptosis in a Human Colon Carcinoma Cell Line. J. Cell Biol. 138, 449 (1997).
49. S. Desagher, J.-C. Martinou, Mitochondria as the central control point of apoptosis. Trends Cell Biol. 10, 369-377 (2000).
50. S. Chauhan et al., Pharmaceutical screen identifies novel target processes for activation of autophagy with a broad translational potential. Nat. Commun. 6, 8620 (2015).
51. Y Yokoyama et al., Synergy between Angiostatin and Endostatin: Inhibition of Ovarian Cancer Growth. Cancer Res. 60, 2190-2196 (2000).
52. W. Tao et al., Two-Dimensional Antimonene-Based Photonic Nanomedicine for Cancer Theranostics. Adv. Mater. 30, 1802061 (2018).
53. K. Bensaad et al., TIGAR, a p53-Inducible Regulator of Glycolysis and Apoptosis. Cell 126, 107-120 (2006).
54. J.-M. Xie et al., TIGAR Has a Dual Role in Cancer Cell Survival through Regulating Apoptosis and Autophagy. Cancer Res. 74, 5127 (2014).
55. K. Bensaad, E. C. Cheung, K. H. Vousden, Modulation of intracellular ROS levels by TIGAR controls autophagy. EMBO J. 28, 3015-3026 (2009).
56. X. Sui et al., p53 signaling and autophagy in cancer: a revolutionary strategy could be developed for cancer treatment. Autophagy 7, 565-571 (2011).
57. D. R. Green, G. Kroemer, Cytoplasmic functions of the tumour suppressor p53. Nature 458, 1127-1130 (2009).
58. D. Meley et al., AMP-activated protein kinase and the regulation of autophagic proteolysis. J. Biol. Chem. 281, 34870-34879 (2006).
59. K. Schulze et al., Exome sequencing of hepatocellular carcinomas identifies new mutational signatures and potential therapeutic targets. Nat. Genet. 47, 505-511 (2015).
60. B. Vogelstein, D. Lane, A. J. Levine, Surfing the p53 network. Nature 408, 307-310 (2000).
61. w. b. e. Cancer Genome Atlas Research Network. Electronic address, N. Cancer Genome Atlas Research, Comprehensive and Integrative Genomic Characterization of Hepatocellular Carcinoma. Cell 169, 1327-1341 e1323 (2017).
62. N. Cancer Genome Atlas Research, Comprehensive molecular profiling of lung adenocarcinoma. Nature 511, 543-550 (2014).
63. A. Ventura et al., Restoration of p53 function leads to tumour regression in vivo. Nature 445, 661 (2007).
64. A. Mandinova, S. W. Lee, The p53 pathway as a target in cancer therapeutics: obstacles and promise. Sci. Transl. Med. 3, 64rv61 (2011).
65. M. Harajly et al., p53 Restoration in Induction and Maintenance of Senescence: Differential Effects in Premalignant and Malignant Tumor Cells. Mol. Cell. Biol. 36, 438-451 (2016).
66. S. Zhang et al., Small-Molecule NSC59984 Restores p53 Pathway Signaling and Antitumor Effects against Colorectal Cancer via p73 Activation and Degradation of Mutant p53. Cancer Res. 75, 3842-3852 (2015).
67. D. J. Panka, Q. Liu, A. K. Geissler, J. W. Mier, Effects of HDM2 antagonism on sunitinib resistance, p53 activation, SDF-1 induction, and tumor infiltration by CD11b+/Gr-1+ myeloid derived suppressor cells. Mol. Cancer 12, 17 (2013).
68. A. P. Turnbull et al., Molecular basis of USP7 inhibition by selective small-molecule inhibitors. Nature 550, 481-486 (2017).
69. V. J. N. Bykov, G. Selivanova, K. G. Wiman, Small molecules that reactivate mutant p53. Eur. J. Cancer 39, 1828-1834 (2003)..
70. B. A. Foster, H. A. Coffey, M. J. Morin, F. Rastinejad, Pharmacological rescue of mutant p53 conformation and function. Science 286, 2507-2510 (1999).
71. M. Andreeff et al., Results of the Phase I Trial of RG7112, a Small-Molecule MDM2 Antagonist in Leukemia. Clin. Cancer Res. 22, 868-876 (2016).
72. V. A. de Weger et al., A phase I study of the HDM2 antagonist SAR405838 combined with the MEK inhibitor pimasertib in patients with advanced solid tumours. Brit. J. Cancer 120, 286-293 (2019).
73. D. A. Sallman et al., Phase Ib/II combination study of APR-246 and azacitidine (AZA) in patients with TP53 mutant myelodysplastic syndromes (MDS) and acute myeloid leukemia (AML). Cancer Res. 78, CT068-CT068 (2018).
74. S. Pearson, H. Jia, K. Kandachi, China approves first gene therapy. Nat. Biotechnol. 22, 3-4 (2004).
75. R. Osborne, Ark floats gene therapy's boat, for now. Nat. Biotechnol. 26, 1057-1059 (2008).
76. G.-x. Chen et al., Clinical utility of recombinant adenoviral human p53 gene therapy: current perspectives. OncoTargets Ther. 7, 1901-1909 (2014).
77. J. Shi, P. W. Kantoff, R. Wooster, O. C. Farokhzad, Cancer nanomedicine: progress, challenges and opportunities. Nat. Rev. Cancer 17, 20-37 (2017).
78. H. Yin et al., Non-viral vectors for gene-based therapy. Nat. Rev. Genet. 15, 541-555 (2014).
79. Q. Xiong et al., Biomedical applications of mRNA nanomedicine. Nano Res. 11, 5281-5309 (2018).
80. M. A. Islam et al., Biomaterials for mRNA delivery. Biomater. Sci. 3, 1519-1533 (2015).
81. K. A. Hajj, K. A. Whitehead, Tools for translation: non-viral materials for therapeutic mRNA delivery. Nat. Rev. Mater. 2, 17056 (2017).
82. M. G. Prieve et al., Targeted mRNA Therapy for Ornithine Transcarbamylase Deficiency. Mol. Ther. 26, 801-813 (2018).
83. E. Schrom et al., Translation of Angiotensin-Converting Enzyme 2 upon Liver- and Lung-Targeted Delivery of Optimized Chemically Modified mRNA. Mol. Ther. Nucleic Acids 7, 350-365 (2017).
84. S. Ramaswamy et al., Systemic delivery of factor IX messenger RNA for protein replacement therapy. Proc. Natl. Acad. Sci. USA 114, E1941-E1950 (2017).
85. D. An et al., Systemic Messenger RNA Therapy as a Treatment for Methylmalonic Acidemia. Cell Rep. 21, 3548-3558 (2017).
86. J. B. Miller et al., Non-Viral CRISPR/Cas Gene Editing In Vitro and In Vivo Enabled by Synthetic Nanoparticle Co-Delivery of Cas9 mRNA and sgRNA. Angew. Chem.-Int. 56, 1059-1063 (2017).
87. C. Jiang et al., A non-viral CRISPR/Cas9 delivery system for therapeutically targeting HBV DNA and pcsk9 in vivo. Cell Res. 27, 440-443 (2017).
88. B. Li et al., An Orthogonal Array Optimization of Lipid-like Nanoparticles for mRNA Delivery in Vivo. Nano Lett. 15, 8099-8107 (2015).
89. P. Castellani, E. Balza, A. Rubartelli, Inflammation, DAMPs, tumor development, and progression: a vicious circle orchestrated by redox signaling. Antioxid. Redox Signal. 20, 1086-1097 (2014).
90. N. Carayol et al., Critical roles for mTORC2- and rapamycin-insensitive mTORC1-complexes in growth and survival of BCR-ABL-expressing leukemic cells. Proc. Natl. Acad. Sci. USA 107, 12469 (2010).
91. E. Tasdemir et al., A dual role of p53 in the control of autophagy. Autophagy 4, 810-(2008).
92. P. M. Valencia, O. C. Farokhzad, R. Karnik, R. Langer, Microfluidic technologies for accelerating the clinical translation of nanoparticles. Nat. Nanotechnol. 7, 623-629 (2012).
93. J.-M. Lim et al., Ultra-High Throughput Synthesis of Nanoparticles with Homogeneous Size Distribution Using a Coaxial Turbulent Jet Mixer. ACS Nano 8, 6056-(2014).
94. Y Kim et al., Mass production and size control of lipid-polymer hybrid nanoparticles through controlled microvortices. Nano Lett. 12, 3587-3591 (2012).
95. M. A. Miller et al., Predicting therapeutic nanomedicine efficacy using a companion magnetic resonance imaging nanoparticle. Sci. Transl. Med. 7, 314ra183-314ra183 (2015).
96. A. Ventura et al., Extracorporeal Photochemotherapy Drives Monocyte-to-Dendritic Cell Maturation to Induce Anticancer Immunity. Cancer Res. 78, 4045-4058 (2018).
97. Ning, Y C.; Hui, N.; Qing, B.; Zhuo, Y M.; Sun, W.; Du, Y; Liu, S. L.; Liu, K. L.; Zhou J. L. ZCCHC10 Suppresses Lung Cancer Progression and Cisplatin Resistance by Attenuating MDM2-Mediated p53 Ubiquitination and Degradation. Cell Death and Disease, 2019, 10, 414-425,
98. Choi, H. S.; Kim, Y K.; Yun P. Y The Role of p53 Status on the Synergistic Effect of CKD-602 and Cisplatin on Oral Squamous Cell Carcinoma Cell Lines. Molecular Biology Reports, 2019, 46, 617-625.
99. Yang, X. M.; Zhang, Q.; Yang, X. F.; Zhao, M. Y; Yang, T.; Yao, A. M.; Tian, X. F. PACT Cessation Overcomes Ovarian Cancer Cell Chemoresistance to Cisplatin by Enhancing p53-mediated Apoptotic Pathway. Biochemical and Biophysical Research Communications, 2019, 511, 719-724.
100. Han, J. Y; Chung, Y J.; Park, S. W.; Kim, J. S.; Rhyu, M. G.; Kim, H. K.; Lee, K. S.; The Relations between Cisplatin-Induced Apoptosis and p53, bcl-2 and bax Expression in Human Lung Cancer Cells. The Korean Journal of Internal Medicine. 1999, 14, 42-52.
101. Rusch, V; Klimstra, D.; Venkatraman, E.; Oliver, J.; Martini, N.; Gralla, Richard.; Kris, M.; Dmitrovsky, Ethan. Aberrant p53 Expression Predicts Clinical Resistance to Cisplatin-Based Chemotherapy in Locally Advanced Non-Small Cell Lung Cancer, Cancer Research, 1995, 55, 5038-5042.
102. Freed-Pastor W.; Prives, C.; Targeting Mutant p53 through the Mevalonate Pathway. Nature Cell Biology. 2016, 18, 1122-1124.
103. Du, W., Jiang, P., Mancuso, A., Stonestrom, A., Brewer, M. D.; Minn, A. J, Mak, T. W.; Wu, M.; Yang, X. TAp73 enhances the pentose phosphate pathway and supports cell proliferation. Nature Cell Biology. 2013, 15, 991-1000.
104. Lima, S.; Takabe, K.; Newton, J.; Saurabh, K.; Young, M. M.; Leopoldino, A. M.; Hait, N. C.; Roberts J. L.; Wang, H. G.; Dent, P.; Milstien, S.; Booth, L.; Spiegel, S. TP53 is Required for BECN1- and ATG5-Dependent Cell Death Induced by Sphingosine Kinase 1 Inhibition. Autophagy, 2018, 14, 1-16.
105. Walerych, D.; Lisek, K.; Sommaggio, R.; Piazza, S.; Ciani, Y; Dalla, E.; Rajkowska, K.; Gaweda-Walerych, K.; Ingallina, E.; Tonelli, C.; Morelli, M. J.; Amato, A.; Eterno, V.; Zambelli, A.; Rosato, A.; Amati, B.; Wisniewski, J. R.; Del Sal, G. Proteasome Machinery is Instrumental in a Common Gain-of-Function Program of the p53 Missense Mutants in Cancer. Nature Cell Biology, 2016, 18, 897-909.
106. Fantauzzo, K. A.; Soriano, P.; PI3K-Mediated PDGFRa Signaling Regulates Survival and Proliferation in Skeletal Development Through p53-Dependent Intracellular Pathways. Genes & Development, 2014, 28, 1005-1017.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A method of treating a cancer, the method comprising administering to a subject in need thereof a therapeutically effective amount of an mRNA encoding tumor suppressor protein p53 in combination with an anticancer therapeutic agent, or a pharmaceutically acceptable salt thereof, wherein the anticancer therapeutic agent is selected from an mTOR inhibitor, a platinum-based antineoplastic agent, and an AMPK activating agent.

2. The method of claim 1, wherein the p53-encoding mRNA is within a delivery vehicle capable of providing release of the p53-encoding mRNA in the cancer cell.

3. The method of claim 2, wherein the delivery vehicle is a particle comprising:
a water-insoluble polymeric core; and
the p53-encoding mRNA and a complexing agent within the core.

4. The method of claim 3, wherein the particle further comprises a shell comprising at least one amphiphilic material surrounding the water-insoluble polymeric core.

5. The method of claim 2, wherein the water-insoluble polymeric core comprises one or more polymers selected from a poly(lactic acid), a poly(glycolic acid), and a copolymer of lactic acid and glycolic acid.

6. The method of claim 2, wherein the water-insoluble polymer comprises at least one repeating unit according to Formula (I) or Formula (II): 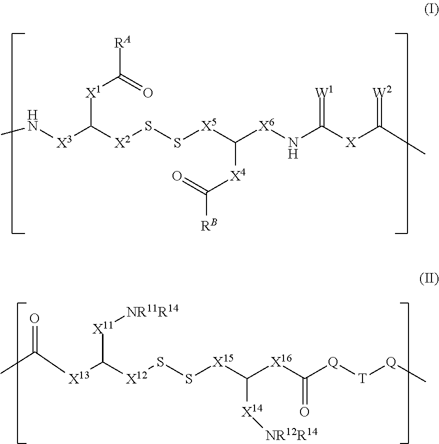
wherein:
X¹ is a bond or C₁₋₁₀₀ alkylene;
X² is C₁₋₁₀₀ alkylene;
X³ is a bond or C₁₋₁₀₀ alkylene;
X⁴ is a bond or C₁₋₁₀₀ alkylene;
X⁵ is C₁₋₁₀₀ alkylene;
X⁶ is a bond or C₁₋₁₀₀ alkylene;
R^{A} is OR¹ or NR³R⁴;
R^{B} is OR² or NR²R⁴;
R¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
R² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
each R³ is independently H, C₁₋₁₀₀ alkyl or C(═O)R⁶;
each R⁴ is independently H or C₁₋₁₀₀ alkyl;
each R⁵ is independently H or C₁₋₁₀₀ alkyl;
each R⁶ is independently H or C₁₋₁₀₀ alkyl;
W¹ is O, S, or NH;
W² is O, S, or NH;
X is C₁₋₁₀₀ alkylene, C₂₋₁₀₀ alkenylene, or C₂₋₁₀₀ alkynylene;
provided that when W¹ and W² are both O, then X is C₃₋₁₀₀ alkylene, C₂₋₁₀₀ alkenylene, or C₂₋₁₀₀ alkynylene;
each m is 0, 1 or 2;
X¹¹ is a bond or C₁₋₁₀₀ alkylene;
X¹² is C₁₋₁₀₀ alkylene;
X¹³ is a bond or C₁₋₁₀₀ alkylene;
X¹⁴ is a bond or C₁₋₁₀₀ alkylene;
X¹⁵ is C₁₋₁₀₀ alkylene;
X¹⁶ is a bond or C₁₋₁₀₀ alkylene;
R¹¹ is H, C₁₋₁₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;
R¹² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;
each R¹³ is independently H, C₁₋₁₀₀ alkyl or C(═O)R¹⁶;
each R¹⁴ is independently H or C₁₋₁₀₀ alkyl;
each R¹⁵ is independently H or C₁₋₁₀₀ alkyl;
each R¹⁶ is independently H or C₁₋₁₀₀ alkyl;
each Q is independently O or NR¹⁷;
each R¹⁷ is H or C₁₋₁₀₀ alkyl;
T is C₂₋₁₀₀ alkylene, C₄₋₁₀₀ alkenylene, or C₄₋₁₀₀ alkynylene; and
each n is 0, 1 or 2.

7. The method of claim 6, wherein the water-insoluble polymer comprises at least one repeating unit according to Formula (I), wherein:
X¹ is a bond or C₁₋₄ alkylene;
X² is C₁₋₄ alkylene;
X³ is a bond or C₁₋₄ alkylene;
X⁴ is a bond or C₁₋₄ alkylene;
X⁵ is C₁₋₄ alkylene;
X⁶ is a bond or C₁₋₄ alkylene;
R^{A} is OR¹ or NR⁴R⁴;
R^{B} is OR² or NR²R⁴;
R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C1-20 alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
each R³ is independently H, C₁₋₆ alkyl or C(═O)R⁶;
each R⁴ is independently H or C₁₋₆ alkyl;
each R⁵ is independently H or C₁₋₆ alkyl;
each R⁶ is independently H or C₁₋₆ alkyl;
W¹ is O, S, or NH;
W² is O, S, or NH;
X is C₂₋₂₀ alkylene, C₂₋₂₀ alkenylene, or C₂₋₂₀ alkynylene;
provided that when W¹ and W² are both O, then X is C₃₋₂₀ alkylene, C₂₋₂₀ alkenylene, or C₂₋₂₀ alkynylene; and
each m is 0, 1 or 2.

8. The method of claim 6, wherein the water-insoluble polymer comprises at least one repeating unit according to Formula (Ia): 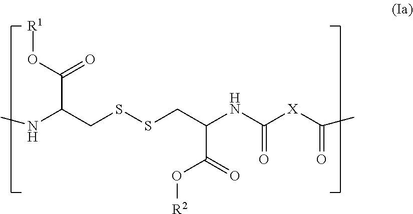
wherein:
R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
each R³ is independently H, C₁₋₆ alkyl or C(═O)R⁶;
each R⁴ is independently H or C₁₋₆ alkyl;
each R⁵ is independently H or C₁₋₆ alkyl;
each R⁶ is independently H or C₁₋₆ alkyl;
X is C₃₋₂₀ alkylene, C₂₋₂₀ alkenylene, or C₂₋₂₀ alkynylene; and
each m is 0, 1 or 2.

9. The method of claim 8, wherein:
R¹ is H, C₁₋₂₀ alkyl, C₂₋₂₀ alkenyl, C₂₋₂₀ alkynyl, C₃₋₁₀ cycloalkyl, or C₆₋₁₀ aryl;
R² is H, C₁₋₂₀ alkyl, C₂₋₂₀ alkenyl, C₂₋₂₀ alkynyl, C₃₋₁₀ cycloalkyl, or C₆₋₁₀ aryl; and
X is C₃₋₂₀ alkylene.

10. The method of claim 8, wherein:
R¹ is H or C₁₋₆ alkyl;
R² is H or C₁₋₆ alkyl; and
X is C₄₋₁₀ alkylene.

11. The method of claim 8, wherein the at least one repeating unit has the structure selected from:

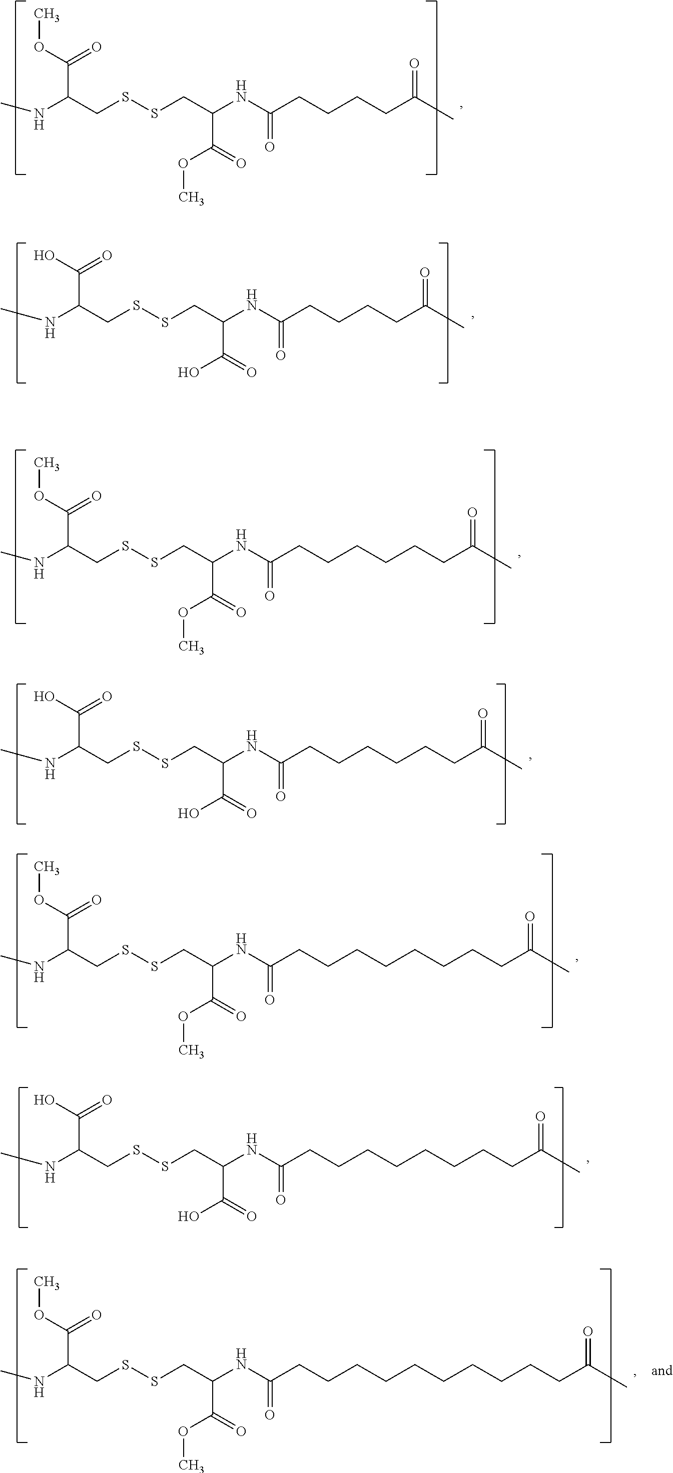

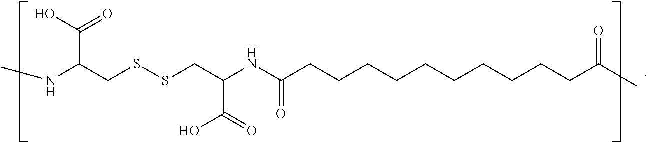

12. The method of claim 3, wherein the complexing agent is a cationic lipid or a cationic lipid-like material such as lipophilic moiety-modified amino dendrimer.

13. The method of claim 12, the cationic lipid is selected from 1,2-dioleoyl-3-trimethylammonium-propane (DOTAP) and 1,2-di-O-octadecenyl-3-trimethylammonium propane (DOTMA); and the lipophilic moiety-modified amino dendrimer is selected from polypropylenimine tetramine dendrimer generation 1 modified with a lipophilic moiety, ethylenediamine core-poly (amidoamine) (PAMAM) generation 0 dendrimer (G0) modified with C14 (G0-C14 dendrimer); and ethylenediamine branched polyethyleneimine modified with a lipophilic moiety.

14. The method of claim 3, wherein the weight ratio of the complexing agent to the p53-encoding mRNA in the core of the particle is from about 5 to about 20.

15. The method of claim 4, wherein the amphiphilic material comprises one or more compounds selected from neutral, cationic and anionic lipids, PEG-phospholipid, and a PEG-ceramide.

16. The method of claim 15, wherein the amphiphilic material comprises 1,2-dimyristoyl-sn-glycero-3-phosphoethanolamine-N-[methoxy(polyethylene glycol)] (DMPE-PEG) or 1,2-distearoyl-sn-glycero-3-phosphoethanolamine-N-[methoxy(polyethylene glycol)](DSPE-PEG), or a combination thereof.

17. The method of claim 1, wherein the mTOR inhibitor is everolimus, or a pharmaceutically acceptable salt thereof.

18. The method of claim 1, wherein the platinum-based antineoplastic agent is cisplatin, or a pharmaceutically acceptable salt thereof.

19. The method of claim 1, wherein the AMPK activating agent is metformin, or a pharmaceutically acceptable salt thereof.

20. The method of claim 1, wherein the cancer is selected from lung cancer and liver cancer.
